(19) United States
(12) Patent Application Publication
(10) Pub. No.: US 2019/0142713 A1
(43) Pub. Date: May 16, 2019

(54) Optical Evanescent Color Change Compositions and Methods of Making and Using the Same

(71) Applicant: Segan Industries, Inc.

(72) Inventors: Hans O. Ribi, Hillsborough, CA (US); Sanchita Biswas, Dublin, CA (US)

(21) Appl. No.: 16/092,332

(22) PCT Filed: Apr. 13, 2017
(86) PCT No.: PCT/US17/27461

§ 371 Date: Oct. 9, 2018

Related U.S. Application Data
(60) Provisional application No. 62/322,152, filed on Apr. 13, 2016.
(60) Provisional application No. 62/411,140, filed on Oct. 21, 2016.

(51) Int. Cl. A61K 8/11; A61K 8/81; A61K 8/84

(52) U.S. Cl. CPC A61K 8/11; A61Q 1/02; A61K 8/84; A61K 8/8135

(57) ABSTRACT

Aspects of the invention include evanescent color change compositions having a colored pigment and a color changer that changes the colored pigment from colored to colorless in response to an applied stimulus to the color changer. Methods for preparing the subject evanescent color change compositions and liquid compositions (e.g., sunscreens) that include the subject evanescent color change compositions are also described. Methods for forming a uniform coating on a surface using the subject evanescent color change compositions are also provided.

### CROSS-REFERENCE TO RELATED APPLICATIONS

Pursuant to 35 U.S.C. § 119(e), this application claims priority to the filing dates of U.S. Provisional Application Ser. No. 62/322,152 filed on Apr. 13, 2016 and U.S. Provisional Application Ser. No. 62/411,140 filed on Oct. 21, 2016; the disclosures of which applications are herein incorporated by reference.

### INTRODUCTION

Skin cancer is the most common type of cancer. It generally forms on skin that has been exposed (sometimes excessively) to electromagnetic radiation, in particular sunlight and ultraviolet light. Skin cancer begins in the epidermis (outer layer), which is made up of squamous cells, basal cells, and melanocytes. Squamous cell and basal cell skin cancers are sometimes called nonmelanoma skin cancers. Nonmelanoma skin cancer is typically less aggressive and rarely spreads to other parts of the body. Melanoma is more aggressive and if not diagnosed early, can invade nearby tissues and spread throughout the body.

The most common method of protecting the skin from overexposure to the radiation of the sun is a sunblock or a sunscreen. In general, sunscreens are opaque or nearly opaque creams or lotions that protect your skin by absorbing or reflecting UV radiation (e.g., UV-A and UV-B radiation). Commercial sunscreens have a Sun Protection Factor (SPF) rating. The SPF rating indicates how long a sunscreen remains effective at protecting the skin from the radiation from the sun. The duration that the sunscreen will be effective is function of the SPF factor and the normal length of time it takes for skin to burn in the absence of the applied sunscreen. To ensure proper protection from the sun, the sunscreen should be applied uniformly in a sufficiently thick layer to the skin.

### SUMMARY

Aspects of the invention include evanescent color change compositions having a colored pigment and a color changer that changes the color of the colored pigment, such as from colored to colorless in response to an applied stimulus to the color changer. Aspects of the invention also include evanescent color change compositions having a colored pigment and a color changer that changes the colored pigment from a first color to a second color in response to an applied stimulus to the color changer. Methods for preparing the subject evanescent color change compositions and liquid compositions (e.g., sunscreens) that include the subject evanescent color change compositions are also described. Methods for forming a uniform coating on a surface using the subject evanescent color change compositions are also provided.

In embodiments, evanescent color change compositions include a colored pigment. The colored pigment may be a dye, such as a hydrophobic dye including anthraquinone dyes and other oil soluble dyes. In certain embodiments, the colored pigment is an FD&C dye (i.e., colorants that have been approved by the Food and Drug Administration for use in food, drugs and cosmetics) or a D&C dye (i.e., colorants that have been approved by the Food and Drug Administration for use in drugs and cosmetics). The colored pigment may be a carotenoid. In some embodiments, the colored pigment is chemically modified, such as with a long chain hydrocarbon or a polyalkylene glycol (e.g., polyethylene glycol, polypropylene glycol, etc.).

In embodiments, the evanescent color change compositions include a color-changing pigment which changes from a first color to a second color. The color change exhibited may be any desired chromic transition. For example, colored to colorless transitions may include, but are not limited to: yellow to colorless, orange to colorless, red to colorless, pink to colorless, magenta to colorless, purple to colorless, blue to colorless, turquoise to colorless, green to colorless, brown to colorless and black to colorless among other colored to colorless transitions. Colorless to colored transitions may include, but are not limited to: colorless to yellow, colorless to orange, colorless to red, colorless to pink, colorless to magenta, colorless to purple, colorless to blue, colorless to turquoise, colorless to green, colorless to brown and colorless to black, among other colorless to colored transitions. In some embodiments, the chromic transition is a change from a first color to a second, different color. For example, color to color transitions may include, but are not limited to: orange to yellow, orange to pink, orange to very light green, orange to peach; red to yellow, red to orange, red to pink, red to light green, red to peach; magenta to yellow, magenta to orange, magenta to pink, magenta to light green, magenta to light blue; purple to red, purple to pink, purple to blue; blue to pink; blue to light green, dark blue to light yellow, dark blue to light green, dark blue to light blue; turquoise to light green, turquoise to light blue, turquoise to light yellow, turquoise to light peach, turquoise to light pink; green to yellow, dark green to orange, dark green to light green, dark green to light pink; brown and black to a variety of assorted colors, among other color to color transitions.

In some embodiments, the colored pigment changes colors (e.g., from colored to colorless) in response to the application of light to the color changer. In embodiments, the light may be UV light (e.g., 200 nm to 390 nm), visible light (e.g., 390 nm to 700 nm) or infrared light (e.g., 700 nm to 1200 nm). The some instances, the color changer is a free radical photoinitiator, such as a phosphine oxide photoinitiator, an α-amino ketone photoinitiator, a titanocene photoinitiator and an azide photoinitiator. In other instances, the color changer is a singlet oxygen photosensitizer, such as a polycyclic aromatic hydrocarbon or a compound that absorbs light of 400 nm or more, such as Rose Bengal, fluorescein, eosin blue, erythrosine B, methylene blue, acridine or a combination thereof. In certain instances, the color changer initiates the decomposition of the colored pigment in response to the applied stimulus (e.g., light)

Evanescent color change compositions may further include additional coloring agents, thermochromic compounds, photochromic compounds and the like, in addition to various excipients, such as anti-oxidants, fillers, preservatives, plasticizers, softening or hardening agents, adhesives, tackifying agents, viscosity modifiers, resins, buffers, among other excipients. Evanescent color change compositions of interest may be microencapsulated systems where one or both of the colored pigment and color changer are microencapsulated.

Aspects of the invention also include liquid evanescent color change compositions having a solvent. In certain embodiments, the liquid evanescent color change composition is a sunscreen composition having one or more the subject evanescent color change compositions and a dispersion with a UV absorber. The subject color change sunscreen compositions may include a UV-A light absorber UV-B light absorber or a UV-C light absorber or a mixture thereof. In some embodiments, the sunscreen composition includes a free radical photoinitiator that initiates the decomposition of the colored pigment in response to absorption of UV light. In other embodiments, the sunscreen composition includes a free radical photoinitiator that initiates the decomposition of the colored pigment in response to absorption of visible light. In certain embodiments, the sunscreen composition includes a singlet oxygen photosensitizer that initiates the decomposition of the colored pigment in response to absorption of light of 400 nm or more.

Methods for preparing the subject color change compositions are also described. In embodiments, methods include combining a colored pigment with a color changer that changes the color of the colored pigment (e.g., from colored to colorless) upon application of an applied stimulus to the color changer. In some instances, methods include microencapsulating the colored pigment and the color changer. Methods also include preparing a color change sunscreen composition by combining a dispersion having a UV absorber with a color change composition that includes a colored pigment and a color changer that changes the colored pigment from colored to colorless in response to light.

Aspects of the present disclosure according to certain embodiments described herein include novel ways to visualize application and spreading of sunscreens, lotions, topical emollients through color uniformity using approved FD&C dyes or natural dyes and the like where after application and spreading the color will disappear, fade, or evanesce due to exposure to UV and/or visible light. The subject compositions described herein may include an FD&C dye or natural dye that are approved for food, drug, and skin contact along with a color changer that would cause the color of the colored pigment disappear or change naturally in sunlight. In certain embodiments, the present disclosure enables uniform application of the sunscreen/lotion/emollient on skin using a visible color whereby the color would disappear within seconds to minutes upon sun or certain visible light. The subject compositions also provide for the use of regulatory approved dyes which can significantly reduce many of the compliance issues associated with drug formulations that do not utilize FD&C approved dyes. The subject compositions, according to certain embodiments, provide for a convenient way to visualize and determine uniform application of the sunscreen/lotion/emollient and within a specified period of time the visualization feature would fade to a natural skin tone or full color loss.

Other devices employing the subject evanescent color change compositions are also described. In some embodiments, devices include the evanescent color change composition applied to at least one surface of a substrate, in certain instances in the shape of a pattern, word or in a machine readable format. For example, the evanescent color change compositions may be employed as an ink printed onto a substrate such as paper, plastic, hard surfaces, soft surfaces, stiff or rigid surfaces, compliant surfaces, printed surfaces, printable surfaces, transparent surfaces, semi-transparent surfaces, opaque surfaces, non-transparent surfaces, skin, finger nails, molded surfaces, flexo-graphic printing surfaces, foam surfaces, expanded plastic surfaces, insulating surfaces, conducting surfaces and conducting ink surfaces. In some embodiments, the subject evanescent color change compositions may be included in inks, plastics, coatings, pharmaceutical products, foods and beverages, promotional materials, cosmetic make-up, hand sanitizers, liquid bandages, arts and crafts, commercial signs, evanescent bill boards and signs, automotive waxes, food service sterilization, UV sterilization indication, commercial and craft paints, adhesives and glues, cleaning agents, industrial coatings, medicinal topical products, lip balms, cloth applied emollients, hair care products, hair removal emollients, polishing agents, finger nail polishes, insect repellant, pain relief dental, dental care products such as tooth paste, printed books, magazines and newspapers, printed fliers, optical evanescent receipts, game pieces, secret messages for advertising, military and defense applications including exposure, toxic waste indication, water contamination and purification indication, radiation exposure indicators, house-hold cleaning and sanitation, free-radical induced chemo-therapeutic and immune stimulating adjuvant compositions, topical acne medications, enhanced and/or accelerated bio-degradable additives.

### BRIEF DESCRIPTION OF THE FIGURES

FIGS. 1a-1b depict an example of 2 different evanescent color change compositions exhibiting color evanescence according to certain embodiments.

FIG. 2 depicts an example of 8 different evanescent color change compositions exhibiting color evanescence according to certain embodiments.

FIG. 3 depicts an example of a blue colored composition exhibiting color evanescence according to certain embodiments.

FIGS. 4a-4b depict an example of 21 microencapsulated blue evanescent color change compositions incorporated into different sunscreens exhibiting color evanescence according to certain embodiments.

FIGS. 5a-5c depict an example of microencapsulated blue evanescent color change compositions incorporated into different sunscreens and exposed to different durations of sunlight according to certain embodiments.

FIG. 6 depict an example of color evanescence of a microencapsulated blue evanescent color change composition over time according to certain embodiments.

FIGS. 7a-7b depict an example of a yellow evanescent color change composition incorporated into an SPF 50 sunscreen and exposed to sunlight according to certain embodiments.

FIGS. 8a-8b depict an example of a D&C green dye evanescent color change composition incorporated into an SPF 50 sunscreen and exposed to sunlight according to certain embodiments.

FIGS. 9a-9b depict an example of a D&C blue dye evanescent color change composition incorporated into an SPF 50 sunscreen and exposed to sunlight on the surface of the skin according to certain embodiments. Before exposure to sunlight (FIG. 9a), the composition is blue.

FIGS. 10a-10c depict an example of a blue evanescent color change composition incorporated into different SPF (30, 50 and 70) sunscreens and exposed to sunlight according to certain embodiments.

FIGS. 11a-11c depict an example of a D&C green dye evanescent color change composition incorporated into an SPF 30 and 50 sunscreens and exposed to sunlight according to certain embodiments.

FIGS. 12a-12e depict an example of a fingernail polish composition having a photochromic dye and an evanescent color change composition according to certain embodiments.

FIGS. 13a-13d depict an example of a surface coating having a photochromic dye and evanescent color change composition according to certain embodiments.

FIGS. 14a-14c depict the use of an evanescent color change composition for coating a printed medium according to certain embodiments.

### DETAILED DESCRIPTION

Aspects of the invention include evanescent color change compositions having a colored pigment and a color changer that changes the color of the colored pigment, such as from colored to colorless, in response to an applied stimulus to the color changer. Aspects of the invention also include evanescent color change compositions having a colored pigment and a color changer that changes the colored pigment from a first color to a second color in response to an applied stimulus to the color changer. Methods for preparing the subject evanescent color change compositions and liquid compositions (e.g., sunscreens) that include the subject evanescent color change compositions are also described. Methods for forming a uniform coating on a surface using the subject evanescent color change compositions are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As reviewed above, the present disclosure provides evanescent color change compositions having a colored pigment and a color changer that changes the color of the colored pigment (e.g., from colored to colorless) in response to an applied stimulus to the color changer as well as methods for preparing the subject evanescent color change compositions. In further describing embodiments of the invention, evanescent color change compositions are first reviewed in greater detail. Next, methods for preparing the subject compositions are described. Liquid compositions (e.g., sunscreens) and other devices that include the evanescent color change compositions are also described.

### Evanescent Color Change Compositions

As summarized above, the subject invention provides evanescent color change compositions having a colored pigment and a color changer that changes the color of the colored pigment, such as from colored to colorless, in response to an applied stimulus to the color changer (e.g., light). By changing from “colored to colorless” is meant that the colored pigment retains 10% or less of its initial color as determined by visual inspection (e.g., by the human eye or a computer employing an optical detector device), such as 7% or less, such as by 5% or less, such as by 4% or less, such as by 3% or less, such as by 2% or less, such as by 1% or less, such as by 0.5% or less, such as by 0.1% or less, such as by 0.01% or less, such as by 0.001% or less and including by 0.0001% or less. In certain embodiments, all of the initial color of the colored pigment is dissipated by the color changer and the pigment exhibits no color (i.e., is no longer visible by the human eye or by a computer employing an optical detector device). In other words, the visible color of the pigment is reduced by the color changer, such as by reducing the visible color of the pigment by 90% or more, such as by 95% or more, such as by 97% or more, such as by 98% or more, such as by 99% or more, such as by 99.5% or more, such as by such as by 99.9% or more, such as by 99.99% or more, such as by 99.999% or more, such as by reducing the visible color of the pigment (e.g., darkness) by 99.9999% or more, including eliminating the visible color of the pigment altogether (i.e., 100%). As described in greater detail below, in certain embodiments the color changer changes the color of the composition by decomposing the colored pigment, such as by free radical initiated decomposition or by singlet oxygen catalyzed decomposition.

In embodiments of the invention, compositions of interest change from colored to colorless (i.e., color evanescence, color disappearance) upon application of an applied stimulus to the color changer. Stimuli sufficient for inducing the color change may be a variety of different types of physicochemical stimuli, depending on the color changers in the subject compositions, as described in greater detail below. As such, the applied stimulus may include but is not limited to light, mechanical perturbation, changes in temperature, change in pH, chemical exposure, biochemical exposure, ionization, state of hydration, state of solvation, hydrogen bonding, protonation. In certain embodiments, the applied stimulus is light. Thus, evanescent color change compositions of interest may be photo-evanescent, mechano-evanescent, thermos-evanescent, solvato-evanescent, hydro-evanescent or halo-evanescent compositions. As reviewed above, evanescent color change compositions of the present invention include a colored pigment and a color changer that changes the color of the colored pigment (e.g., from colored to colorless) in response to an applied stimulus to the color changer. The color change exhibited may be any desired chromic transition. For example, colored to colorless transitions may include, but are not limited to: yellow to colorless, orange to colorless, red to colorless, pink to colorless, magenta to colorless, purple to colorless, blue to colorless, turquoise to colorless, green to colorless, brown to colorless and black to colorless among other colored to colorless transitions. Colorless to colored transitions may include, but are not limited to: colorless to yellow, colorless to orange, colorless to red, colorless to pink, colorless to magenta, colorless to purple, colorless to blue, colorless to turquoise, colorless to green, colorless to brown and colorless to black, among other colorless to colored transitions. In some embodiments, the chromic transition is a change from a first color to a second, different color. For example, color to color transitions may include, but are not limited to: orange to yellow, orange to pink, orange to very light green, orange to peach; red to yellow, red to orange, red to pink, red to light green, red to peach; magenta to yellow, magenta to orange, magenta to pink, magenta to light green, magenta to light blue; purple to red, purple to pink, purple to blue; blue to pink; blue to light green, dark blue to light yellow, dark blue to light green, dark blue to light blue; turquoise to light green, turquoise to light blue, turquoise to light yellow, turquoise to light peach, turquoise to light pink; green to yellow, dark green to orange, dark green to light green, dark green to light pink; brown and black to a variety of assorted colors, among other color to color transitions.

In some embodiments, the color transition is a change in the opacity of the color of the subject composition. For example, the change in opacity may be an increase in opacity by 1% or more, such as by 2% or more, such as by 5% or more, such as by 10% or more, such as by 25% or more, such as by 50% or more, such as by 75% or more, such as by 90% or more, such as by 95% or more, such as by 99% or more and including by 99.99% or more. In certain instances, the color transition is an increase in the opacity of the color change composition by 1.5-fold or more, such as by 2-fold or more, such as by 3-fold or more, such as by 5-fold or more, such as by 10-fold or more and including by 25-fold or more. In other embodiments, the color transition is a decrease in the opacity of the color change composition, such as a decrease in opacity by 1% or more, such as by 2% or more, such as by 5% or more, such as by 10% or more, such as by 25% or more, such as by 50% or more, such as by 75% or more, such as by 90% or more, such as by 95% or more, such as by 99% or more and including by 99.99% or more. In certain instances, the color transition is a decrease in the opacity of the color change composition by 1.5-fold or more, such as by 2-fold or more, such as by 3-fold or more, such as by 5-fold or more, such as by 10-fold or more and including by 25-fold or more.

Depending on the desired effect, the color transition includes both a color change and a change in opacity. For example, the color transition may also include an increase in the opacity of the color of the subject evanescent color change composition, such as an increase in opacity by 1% or more, such as by 2% or more, such as by 5% or more, such as by 10% or more, such as by 25% or more, such as by 50% or more, such as by 75% or more, such as by 90% or more, such as by 95% or more, such as by 99% or more and including by 99.99% or more. In certain embodiments, the color transition includes an increase in the opacity of the color of the subject evanescent color change composition by 1.5-fold or more, such as by 2-fold or more, such as by 3-fold or more, such as by 5-fold or more, such as by 10-fold or more and including by 25-fold or more. In other embodiments, the color transition also includes a decrease in the opacity of the color of the subject evanescent color change composition, such as a decrease in the opacity of the color of the subject evanescent color change composition, such as a decrease in opacity by 1% or more, such as by 2% or more, such as by 5% or more, such as by 10% or more, such as by 25% or more, such as by 50% or more, such as by 75% or more, such as by 90% or more, such as by 95% or more, such as by 99% or more and including by 99.99% or more. In certain embodiments, the color transition includes a decrease in the opacity of the color of the subject evanescent color change composition by 1.5-fold or more, such as by 2-fold or more, such as by 3-fold or more, such as by 5-fold or more, such as by 10-fold or more and including by 25-fold or more.

In certain embodiments, the subject compositions provide for a color transition in which a chemiluminescent compound is activated. In these embodiments, a stimulus is applied to a color changer which activates the chemilumiscent compound to emit light in response to the applied stimulus to the color changer. The chemiluminescent compound may be any suitable compound which emits light as a result of a chemical reaction, including but not limited to, N-(4-Aminobutyl)-N-ethylisoluminol, 4-Aminophthalhydrazide, Coelenterazine, 4,4′-Dianilino-1,1′-binaphthyl-5,5′-disulfonic acid dipotassium salt, N,N′-Dimethyl-9,9′-biacridinium dinitrate, 6-Fmoc-amino-D-luciferin, D-Luciferin, Luciferin 6′-ethyl ether sodium salt, Luminol, 6-(4-Methoxyphenyl)-2-methyl-3,7-dihydroimidazo[1,2-a]pyrazin-3(7H)-one hydrochloride, among other chemiluminescent compounds.

In some embodiments, the color transition (e.g., from a colored state to a colorless state) of the subject compositions is reversible. For example, the evanescent color change composition may reversibly change from a colored state to a colorless state.

In some embodiments, the color change is transient. For example, the subject compositions may change colors and remain in the subsequent color state (e.g., colorless state) for a duration that is 1 second or longer, such as 5 seconds or longer, such as 10 seconds or longer, such as 30 seconds or longer, such as 60 seconds or longer, such as 2 minutes or longer, such as 5 minutes or longer, such as 10 minutes or longer, such as 15 minutes or longer, such as 30 minutes or longer, such as 60 minutes or longer, such as 2 hours or longer, such as 5 hours or longer, such as 12 hours or longer, such as 18 hours or longer and including 24 hours or longer. In these embodiments, after the duration of color change, the color may revert back to the original color state or may change to another color state, such as a second color or transition from the second color to a colorless state. In other embodiments, the transition of the colored pigment from colored to colorless is irreversible. In these embodiments, the evanescent color change composition transitions from a colored state to a colorless state and remains in the colorless state for the remaining lifetime of the evanescent color change composition.

In some embodiments, the colored pigments are dyes, such as a hydrophobic dye. In certain embodiments, the colored pigment is an oil soluble hydrophobic dye such as an anthraquinone dye. Examples of oil-soluble anthraquinone dyes of interest include, but are not limited to D&C Violet 2, D&C Green 6, Solvent Blue 97 (non D&C) CAS 32724-62, and other oil soluble dyes like D&C Yellow 11, D&C Red 17. Natural oil soluble dyes, such as carotenoids, including Beta carotene CAS 7235-40-7, Paprika oleoresin CAS 68917-78-2 and its components: Capsanthin CAS 465-42-9 and Capsorubin CAS 470-38-2; Turmeric dye—Curcumin CAS 458-37-7, Natural Green plant Chlorophyll dye CAS 1406-65-1 may also be included in the subject compositions.

In some embodiments, the colored pigment may be an FD&C dye (i.e., colorants that have been approved by the Food and Drug Administration for use in food, drugs and cosmetics) or a D&C dye (i.e., colorants that have been approved by the Food and Drug Administration for use in drugs and cosmetics). In some embodiments, the colored pigment may be a non-FD&C dye (i.e., colorants that have been not been approved by the Food and Drug Administration for use in food, drugs and cosmetics). Examples of FD&C and D&C dyes of interest include, but are not limited to: FD&C Blue No. 1, FD&C Blue No. 2, D&C Blue No. 4, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 3, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Red No. 39, FD&C Red No. 40(3), D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, Alumina (dried aluminum hydroxide), Annatto extract, Calcium carbonate, Canthaxanthin(3), Caramel, β-Carotene, Cochineal extract, Carmine, Potassium sodium copper chlorophyllin (chlorophyllin-copper complex), Dihydroxyacetone, Bismuth oxychloride, Synthetic iron oxide, Ferric ammonium ferrocyanide, Ferric ferrocyanide, Chromium hydroxide green, Chromium oxide greens, Guanine, Mica-based pearlescent pigments, Pyrophyllite, Mica, Talc, Titanium dioxide, Aluminum powder, Bronze powder, Copper powder, Zinc oxide and combinations thereof.

In some embodiments, the colored pigment is an oil soluble dye. Examples of oil soluble dyes of interest include, but are not limited to:
1) Blue—Solvent Blue 97—with color changer fades fast (3 min at peak hours) to yellowish white.

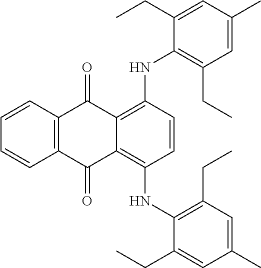 [SMILES: CCc1cc(C)cc(CC)c1Nc1ccc(Nc2c(CC)cc(C)cc2CC)c2c1C(=O)c1ccccc1C2=O]

2) Bluish Violet—D&C Violet 2 (Solvent Blue 90)—with color changer fades fast to grayish white.

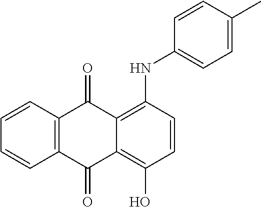 [SMILES: Cc1ccc(Nc2ccc(O)c3c2C(=O)c2ccccc2C3=O)cc1]

3) Bluish Green—D&C Green 6 CAS 128-80-3—with color changer fades fast to grayish white.

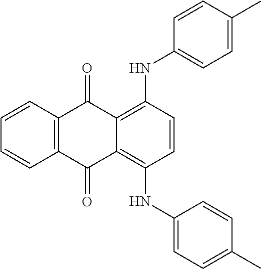 [SMILES: Cc1ccc(Nc2ccc(Nc3ccc(C)cc3)c3c2C(=O)c2ccccc2C3=O)cc1]

4) Orange-red—Natural Paprika oleoresin—with color changer fades fast (1.5 min at peak hours) to perfect white. (as discussed below, was tested several types of oleoresin from 40000cu to 100000cu (cu=color units). 100000cu gives slightly darker red than 2.5 times content of 40000cu, and fades a bit slower.
5) Capsanthin

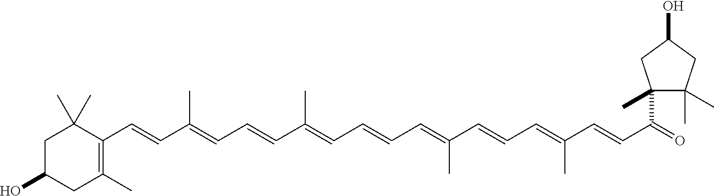

6) Capsorubin

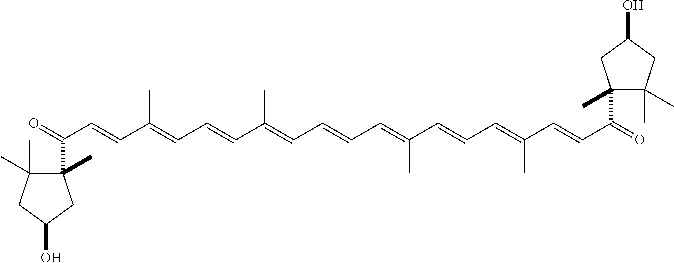

7) Natural carrot oleoresin: Beta carotene—with color changer fades fast to pinkish white.

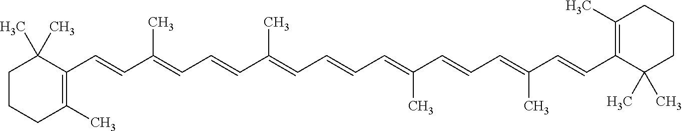 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

8) Turmeric dye: Curcumin—fades under sun exposure fast with color changer and slowly without color changer.

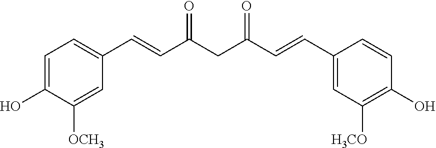 [SMILES: COc1cc(/C=C/C(=O)CC(=O)/C=C/c2ccc(O)c(OC)c2)ccc1O]

9) Natural green plant chlorophyll dye—with color changer fades fast to brown.

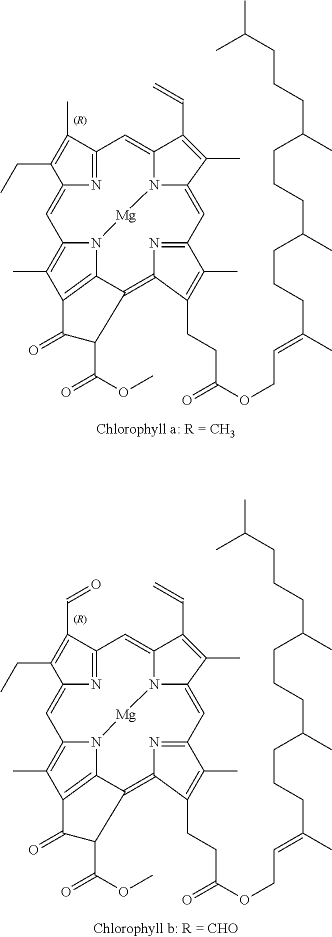

10) D&C Red 17—with color changer fades slowly to dark orange.
11) D&C Yellow 11
12) FD&C Green 8—color change from bright fluorescent yellow to orange in basic conditions.

In some embodiments the colored pigment is a color changing compound, such as a photochromic, mechanochromic, thermochromic, solvatochromic hydrochromic or halochromic compound. Examples of thermochromic dyes of interest include, but are not limited to, bis(2-amino-4-oxo-6-methylpyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the photoproduct 7,7adihydro-4,7,7,7a-tetramethyl-2-phenylbenzo[b]thiophene-5,6-dicarboxylic anhydride, micro-encapsulated dyes, precise melting point compositions, infra-red dyes, spirobenzopyrans, spironnapthooxazines, spirothopyran and related compounds, leuco quinone dyes, natural leuco quinone, traditional leuco quinone, synthetic quinones, thiazine leuco dyes, acylated leuco thiazine dyes, nonacylated leuco thiazine dyes, oxazine leuco dyes, acylated oxazine dyes, nonacylated oxazine leuco dyes, catalytic dyes, combinations with dye developers, arylmethane phthalides, diarylmethane phthalides, monoarylmethane phthalides, monoheterocyclic substituted phthalides, 3-hetercyclic substituted phthalides, diarylmethylazaphthalides, bishetercyclic substituted phthalides, 3,3-bisheterocyclic substituted phthalides, 3-heterocyclic substituted azaphthalides, 3,3-bisheterocyclic substituted azaphthalides, alkenyl substituted phthalides, 3-ethylenyl phthalides, 3,3-bisethylenyl phthalides, 3-butadienyl phthalides, bridged phthalides, spirofluorene phthalides, spirobensanthracene phthalides, bisphthalides, di- and triarylmethanes, diphenylmethanes, carbinol bases, pressure sensitive recording chemistries, photosensitive recording chemistries, fluoran compounds, reaction of keto acids and phenols, reactions of keto acids with 4-alkoxydiphenylamines, reactions of keto acids with 3-alkoxdiphenylamines, reactions of 2′-aminofluorans with aralkyl halides, reaction of 3′-chlorofluorans with amines, thermally sensitive recording mediums, tetrazolium salts, tetrazolium salts from formazans, tetrazolium salts from tetazoles, and the like. Additional thermochromic compounds of interest may include, but are not limited to: light-induced metastable state in a thermochromic copper (II) complex (see e.g., Chem. Commun., 2002, (15), 1578-1579) which under goes a color change from red to purple for a thermochromic complex, [Cu(dieten)2](BF4)2 (dieten=N,N-diethylethylenediamine); bis(2-amino-4-oxo-6-methyl-pyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the photoproduct 7,7adihydro-4,7,7,7a-tetramethyl-2-phenylbenzo[b]thiophene-5,6-dicarboxylic anhydride, and the like.

The amount of colored pigment in the subject composition varies depending on the volume of the composition and amount of color changer present (as described below) and may range from 1% to 30% w/w, such as from 2% to 28% w/w, such as from 3% to 25% w/w, such as from 4% to 23% w/w, such as from 5% to 22% w/w, such as from 7% to 20% w/w, such as from 8% to 18% w/w, such as from 9% to 17% w/w and including from 10% to 15% w/w. In certain embodiments, the molar ratio of colored pigment to color changer ranges from a molar ratio of from 0.01 to 10, such as a molar ratio from 0.05 to 9.5, such as a molar ratio of from 0.1 to 9, such as a molar ratio of from 0.5 to 8.5, such as from a molar ratio from 1 to 8 and including a molar ratio of from 2 to 7.

In certain embodiments, the colored pigment is chemically modified, such as by coupling to a long-chain hydrocarbon. In these embodiments, the long-chain hydrocarbon may be sufficient to convert a water soluble dye into an oil soluble dye. Depending on the chemical structure, the water soluble dye may be coupled to a long-chain hydrocarbon having chain lengths of C5, C6, C7, C8, C9, C10, C11, C12, C13, C14, C15, C16, C18, C19, C20, C21, C22, C23, C24, C25, C26, C27, C28, C29, C30 and longer synthetic and/or naturally derived hydrocarbon chain lengths. In other embodiments, the colored pigment is chemically modified by coupling to a polyalkylene glycol, such as a polyethylene glycol or a polypropylene glycol. In these embodiments, the polymeric length of the polyalkylene glycol is sufficient to convert an oil soluble dye into a water soluble dye. For example, the polymeric length of the polyalkylene glycol may be 2 or more, such as 3 or more, such as 5 or more, such as 7 or more, such as 10 or more, such as 15 or more and including 20 or more.

The solubility of the colored pigment ranges from being greater than 99.999% insoluble in water to being greater than 99.999% soluble in water. Where a solvent is present, as described below, the colored pigment is soluble in the solvent, such as an aqueous soluble, a hydrocarbon solvent, a long chain hydrocarbon alcohol solvent among other types of solvents.

Evanescent color change compositions of interest further include a color changer. In embodiments of the invention, the term “color changer” is used to refer to the component of the evanescent color change composition which initiates and facilitates the color change transition of the composition (e.g., from a colored state to colorless state). In embodiments, the transition (such as from a colored to colorless state) may occur in 120 minutes or less, such as 90 minutes or less, such as 60 minutes or less, such as 30 minutes or less, such as 15 minutes or less, such as 10 minutes or less, such as 5 minutes or less, such as 4 minutes or less, such as 3 minutes or less, such as 2 minutes or less, such as 1 minute or less, such as 0.5 minutes or less and including 0.1 minutes or less.

In embodiments, the evanescent color change composition transitions from a first color to a second color (e.g., from a colored to a colorless state) in response to an applied stimulus to the color changer. Stimuli sufficient for inducing the transition may be a variety of different types of physicochemical stimuli, including but not limited to light, mechanical perturbation, changes in temperature, change in pH, chemical exposure, biochemical exposure, ionization, state of hydration, state of solvation, hydrogen bonding, protonation.

In certain embodiments, the evanescent color change composition transitions from a first color to a second color (e.g., from a colored state to a colorless state) in response is applying light to the color changer. To transition the evanescent color change composition, the stimulus may be applied continuously or in discrete intervals. In some embodiments, the stimulus (e.g., light) is applied to the color changer continuously. In other embodiments, the stimulus is applied in discrete intervals, such 2 or more discrete intervals, such as 3 or more discrete intervals, such as 5 or more discrete intervals and including 10 or more discrete intervals. In some embodiments, evanescent color change composition transitions from colored to colorless concurrently while the stimulus is being applied. In other embodiments, the evanescent color change composition color transitions a predetermined duration after the stimulus has been applied, such as 1 second or more after applying the stimulus, such as 5 seconds or more, such as 10 seconds or more, such as 30 seconds or more and including 60 seconds or more. In certain embodiments, the subject evanescent color change compositions color transitions (e.g., from colored to colorless) after a single application of the stimulus.

In certain instances, the color transition of the subject evanescent color change compositions is initiated with visible light, UV-A light, UV-B light or UV-C light. For example, the color transition of the subject evanescent color change compositions may be initiated by light having a wavelength with ranges from 200 nm to 1200 nm, such as from 250 nm to 1150 nm, such as from 300 nm to 1100 nm, such as from 350 nm to 1050 nm, such as from 400 nm to 1000 nm, such as from 450 nm to 950 nm, such as from 500 nm to 900 nm and including from 550 nm to 850 nm. For example, the wavelength of light used to initiate the color transition (e.g., colored to colorless) may range from 200 nm to 390 nm, from 390 nm to 700 and including from 700 nm to 1200 nm. In certain embodiments, the wavelength of light used to initiate the color transition is 200 nm or greater, such as 225 nm or greater, such as 250 nm or greater, such as 275 nm or greater, such as 300 nm or greater, such as 325 nm or greater, such as 350 nm or greater, such as 375 nm or greater, such as 400 nm or greater, such as 425 nm or greater, such as 450 nm or greater, such as 475 nm or greater, such as 500 nm or greater, such as 550 nm or greater, such as 600 nm or greater, such as 650 nm or greater and including 700 nm or greater.

In some embodiments, the color changer initiates or facilitates the decomposition of the colored pigment. By “decomposition” is meant that the colored pigment undergoes one or more chemical reactions which irreversibly modifies the colored pigment in a manner sufficient to diminish the colored appearance of the colored pigment as determined by visual inspection (e.g., by the human eye or a computer employing an optical detector device), such as by disrupting the chromogenic components (e.g., chromogenic moiety, conjugated π-systems, etc.) of the colored pigment. In embodiments, 5% or more of the colored pigment may be decomposed in response to the applied stimulus to the color changer, such as 10% or more, such as 15% or more, such as 25% or more, such as 50% or more, such as 75% or more, such as 90% or more, such as 95% or more, such as 99% or more and including 99.9% or more. In certain embodiments, all of the colored pigment is decomposed in response to the applied stimulus to the color changer.

In some embodiments, the color changer is a free radical initiator. In some embodiments, the color changer is a photoinitiator such as a photoinitiator which initiates a free radical reaction that chemically reacts with (e.g., decomposes) the colored pigment. Photoinitiators of interest are compounds which produce one or more radical species in response electromagnetic irradiation, such as a phosphine oxide, an α-amino ketone photoinitiator, a titanocene or an azide compound, amines (including aminoaldehydes and aminosilanes), amides (including phosphoramides), ethers (including thioethers), ureas (including thioureas), ferrocene, sulfinic acids and their salts, salts of ferrocyanide, ascorbic acid and its salts, dithiocarbamic acid and its salts, salts of xanthates, salts of ethylene diamine tetraacetic acid and salts of tetraphenylboronic acid. The free radical initiator can be unsubstituted or substituted with one or more non-interfering substituents. In certain instances, the free radical initiator is a peroxide, such as lauryl peroxide, tributyl hydroperoxide and benzoyl peroxide. In other instances, free radical initiators include ionic dye-counterion complex initiators having a borate anion and a complementary cationic dye.

Photoinitiators of interest, in certain embodiments, contain an electron donor atom such as a nitrogen, oxygen, phosphorus, or sulfur atom, and an abstractable hydrogen atom bonded to a carbon or silicon atom alpha to the electron donor atom.

In some embodiments, the photoinitiator is a binary or tertiary photointiator composition. For example, a tertiary photoinitiator may include an iodonium salt, a photosensitizer and an electron donor. Examples of suitable iodonium salts include, but are not limited to, diaryl iodonium salts, diphenyliodonium chloride, diphenyliodonium hexafluorophosphate, and diphenyliodonium tetrafluoroboarate. Suitable photosensitizer in photoinitiator compositions may include, but are not limited to, monoketones and diketones that absorb some light within a range of about 450 nm to about 520 nm (e.g., 450 nm to 500 nm), such as camphorquinone, benzil, furil, 3,3,6,6-tetramethylcyclohexanedione, phenanthraquinone, among other cyclic alpha diketones as well as substituted amines, for example, ethyl dimethylaminobenzoate.

Examples of photoinitiators may include, but are not limited to azobisisobutyronitrile (AIBN), benzoyl peroxide, 2,2-dimethoxy-2-phenylacetophenone (DMPA), 2-hydroxy-2-methyl-1-phenylpropanone, Diphenylmethanone, Benzeneacetic acid, a-oxo-, oxydi-2-1-ethanediyl ester, Phenylbis(2,4,6-trimethylbenzoyl)-phosphine oxide, 1,3-Dihydroxyacetone dimer, 4,4′-Azobis(4-cyanovaleric acid), 2,2′-azobis(2-methylpropionitrile), 1,1′-azobis-(cyclohexanecarbonitrile) 2-methyl-1-(4-methylthio)phenyl-2-morpholinyl-1-propanone, 2-benzyl-2-(dimethylamino)-4′-morpholinobutyrophenone, phenylbis(2,4,6-trimethylbenzoyl)phosphine oxide, 1-hydroxycyclohexyl phenyl ketone and 4-(2-hydroxyethoxy)phenyl-(2-hydroxy-2-propyl)ketone, among other photo-initiators. Suitable photosensitizers also include triplet sensitizers of the “hydrogen abstraction” type, such as for example benzophenone and substituted benzophenone and acetophenones such as benzyl dimethyl ketal, 4-acryloxybenzophenone (ABP), 1-hydroxy-cyclohexyl phenyl ketone, 2,2-diethoxyacetophenone and 2,2-dimethoxy-2-phenylacetophenone, substituted alpha-ketols such as 2-methyl-2-hydroxypropiophenone, benzoin ethers such as benzoin methyl ether and benzoin isopropyl ether, substituted benzoin ethers such as anisoin methyl ether, aromatic sulfonyl chlorides such as 2-naphthalene sulfonyl chloride, photoactive oximes such as 1-phenyl-1,2-propanedione-2-(O-ethoxy-carbonyl)-oxime, thioxanthones including alkyl- and halogen-substituted thioxanthonse such as 2-isopropylthioxanthone, 2-chlorothioxanthone, 2,4 dimethyl thioxanone, 2,4 dichlorothioxanone, and 2,4-diethyl thioxanone.

In certain instances, the photoinitiator is a phosphine oxide photoinitiator such as bis(2,6-dimethoxybenzoyl)-(2-methylpropyl)phosphine oxide, bis(2,4,6-trimethylbenzoyl)-(2-methylpropyl)phosphine oxide, bis(2,6-dimethoxybenzoyl)-(2,4,4-trimethylpentyl)phosphine oxide, bis(2,4,6-trimethylbenzoyl)-(2,4,4-trimethylpentyl)phosphine oxide, (2,6-dimethoxybenzoyl)-(2,4,6-trimethylbenzoyl)-(2-methylpropyl)phosphine oxide, phenylbis(3-{[2-(allyloxy)ethoxy]methyl}-2,4,6-trimethylbenzoyl)phosphine oxide, phenylbis{4-[2-(2-Methoxy-ethoxy)-ethoxy]-2,6-dimethyl-benzoyl}phosphine oxide, phenylbis{3-[2-(2-Methoxy-ethoxy)-ethoxymethyl]-2,4,6-trimethyl-benzoyl}phosphine oxide, phenylbis(2,4,6-trimethyl-benzoyl)-4-[2-(2-methoxyethoxy)-ethoxy]phosphine oxide, phenylbis(2,4,6-trimethyl-benzoyl)-4-methoxy-phosphine oxide, phenylbis(2,4,6-trimethylbenzoyl)-phosphine oxide, 2,4,6-trimethylbenzoyldiphenylphosphine oxide, 2,4,6-trimethylbenzoylphenyl phosphinate, bis(2-methylpropyl)(2,6-dimethoxybenzoyl)phosphine oxide, bis(2-methylpropyl)(2,4,6-trimethylbenzoyl)phosphine oxide, 2,4,6-trimethylbenzoylphenylphosphinic acid ethyl ester, bis(2,4,4-trimethylpentyl)(2,6-dimethoxybenzoyl)phosphine oxide, bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide, bis-(2-chloro-1-naphthoyl)-2,5-dimethylphenylphosphine oxide and combinations thereof. In certain embodiments, a tertiary amine reducing agent is used in conjunction with the phosphine oxide initiator. Example tertiary amine reducing agents may include, but are not limited to, ethyl 4-(N,N-dimethylamino)benzoate and N,N-dimethylaminoethyl methacrylate.

Examples of other suitable photoinitiators include benzil, benzoin, benzoin methyl ether, ethyl (4-dimethylamino)benzoate (EDMAB), DL-camphorquinone (CQ) and benzil diketones.

The wavelength of light used to initiate free radical formation with photoinitiators of interest may vary, as desired and may be UV-A, UV-B, UV-C or visible light. For example, the wavelength of light used to initiate free radical formation may range from 200 nm to 1200 nm, such as from 250 nm to 1150 nm, such as from 300 nm to 1100 nm, such as from 350 nm to 1050 nm, such as from 400 nm to 1000 nm, such as from 450 nm to 950 nm, such as from 500 nm to 900 nm and including from 550 nm to 850 nm. For example, the wavelength of light used to initiate free radical formation with photoinitiators of interest may range from 200 nm to 390 nm, from 390 nm to 700 and including from 700 nm to 1200 nm. In certain embodiments, the wavelength of light used to initiate free radical formation is 400 nm or greater, such as 425 nm or greater, such as 450 nm or greater, such as 475 nm or greater, such as 500 nm or greater, such as 550 nm or greater, such as 600 nm or greater, such as 650 nm or greater and including 700 nm or greater.

In other embodiments, the color changer is a chemical initiator, such as a chemical initiator which chemically initiates a free radical reaction to react with (e.g., decompose) the colored pigment. For example, suitable chemical initiators may include, but are not limited to initiators which produce free radicals such as peroxides, aliphatic azo compounds, initiators which produce a positively charged species such as an acid-forming initiator like boron trifluoride, initiators which produce negatively charged species such as metal amides, alkoxides, hydroxides, cyanides, phosphines, amines, as well as organometallic compounds, like alkyllithim compounds, Ziegler catalysts or Grignard reagents.

In yet other embodiments, the color changer is a thermal initiator, such as an initiator which initiates a free radical reaction thermally. For example, thermal free radical initiators may include peroxides or azo compounds, such as in an amount ranging from about 0.01 wt. % to 15 wt. %, such as 0.05 wt. % to 10 wt. %, such as from about 0.1 wt. % to about 5% and including from about 0.5 wt. % to about 4 wt. %. Examples of peroxides and azo compounds of interest include, but are not limited to, dialkyl peroxides such as t-butyl peroxide and 2,2 bis(t-butylperoxy)propane, diacyl peroxides such as benzoyl peroxide and acetyl peroxide, peresters such as t-butyl perbenzoate and t-butyl per-2-ethylhexanoate, perdicarbonates such as dicetyl peroxy dicarbonate and dicyclohexyl peroxy dicarbonate, ketone peroxides such as cyclohexanone peroxide and methylethylketone peroxide, and hydroperoxides such as cumene hydroperoxide and tert-butyl hydroperoxide. Suitable azo compounds may include azobisisobutyronitrile (AIBN) and azobis-(2,4-dimethylvaleronitrile). Temperatures which initiate the free radical reaction varies depending on the type of thermal free radical initiator and may be from 50° C. to 200° C., such as from 60° C. to 190° C., such as from 75° C. to 180° C., such as from 80° C. to 170° C., such as from 85° C. to 160° C., such as from 90° C. to 155° C. and including from 50° C. to 100° C., such as from 50° C. to 90° C., such as from 50° C. to 85° C., such as from 50° C. to 75 and including 50° C. to 70° C.

The subject compositions may include one or more free radical initiator (e.g., free radical photoinitiator, chemical free radical initiator, thermal free radical initiator), such as 2 or more, such as 3 or more, such as 4 or more and including 5 or more free radical initiators.

In some embodiments, the color changer is a singlet oxygen photosensitizer. In these embodiments, the color changer produces singlet oxygen in response to the applied stimulus (e.g., light). In certain instances, the singlet oxygen sensitizer absorbs light that is 400 nm or greater, such as 450 nm or greater, such as 500 nm or greater, such as 550 nm or greater, such as 600 nm or greater, such as 650 nm or greater and including 700 nm or greater. Singlet oxygen photosensitizers of interest include, but are not limited to polycyclic aromatic hydrocarbons, cyanines, flurosceins, anthracenes and porphyrins. For example, single oxygen photosensitizers may include acridine, acetonaphthone, anthra[1,9-bc:4,10-b′c′]dichromene, 9,10-anthracenedipropionate ion, aluminum(III) sulfophthalocyanine, anthracene, angelicin, anthracenesulfonate ion, acetophenone, 9,10-athraquinone, allylthiourea, bacteriochlorophyll a, benzo[1,2,3-kl:4,5,6k′l′]dixanthene, biphenyl, benzophenone, bilirubin, bilirubin dianion, benoxaprofen, β-carotene, cadmium(II) 1-(2-hydroxyphenylazo)-2-naphtholate, chlorophyll a, camphoroquinone, chlorpromazine, 9,10-dicyanoanthracene, 9,10-dimethylanthracene, 4,7-dimethylallopsoralen, 9,10-dimethylbenz[a]anthracene, 1,4-dimethoxy-9,10-diphenylanthracene, 2,5-dimethylfuran, 4,4′-dimethoxythiobenzophenone, 1,8-dinaphthalene thiophene, diacenaphtho[1,2-b:1′,2′-d]thiophene, 3-(3,4-dihydroxyphenyl)alanine, 9,10-diphenylanthracene, 1,4-diphenyl-1,3-butadiene, 1,3-diphenylisobenzofuran, 2,5-diphenylfuran, 1,6-diphenyl-1,3,5-hexatriene, 1,8-diphenyl-1,3,5,7-octatetraene, 2,5-di-tert-butylfuran, eosin (tetrabromofluorescein), erythrosin (tetraiodofluorescein), ergosterol, furfuryl alcohol, fluorescein, heterocoerdianthrone, histidine, hematoporphyrin, hypericin, imidazole, 4′-methoxyacetophenone, methylene blue, mesodiphenylbenzhelianthrene, mesodiphenylhelianthrene, 1-methylnaphthalene, methoxypsoralen, 2-methyl-2-pentene, mesoporphyrin diethyl ester, mesoporphyrin dimethyl ester, 10-Methyl-9-acridinethione, naphthalene, palladium(II) tetraphenylporphyrin, palladium(II) tetrakis(4-sulfonatophenyl)porphyrin, perylene, pheophytin a, protoporphyrin, protoporphyrin dimethyl ester, 2,7,12,17-tetrapropylporphycene, platinum(II) diazido(2,2′-bipyridine), platinum(II) (1,10-phenanthroline)(tert-butylcatechol), platinum(II) (1,10-phenanthroline)(2,3-naphthalenediol), pyrene, phenazine, Rose Bengal (tetrachlorotetraiodofluorescein), Rose Bengal ethyl ester, retinal, riboflavin, N,N-dimethyl-4-nitrosoaniline, rubrene (5,6,11,12-tetraphenylnaphthacene), 2,2,6,6-tetramethylpiperidin-4-ol, tetracene, tetra(3-hydroxyphenyl)porphyrin, tetra(4-hydroxyphenyl)porphyrin, 2,3-Dimethyl-2-butene (tetramethylethylene), tetra(4-N-methylpyridyl)porphyrin, tetraphenylbacteriochlorin, tetraphenylcyclopentadienone, tetraphenylporphyrin, tetra(4-sulfonatophenyl)porphyrin, tryptophan, uroporphyrin I, zinc(II) tetraphenylporphyrin, zinc(II) 2-(4,5-diphenylimidazol-2-yl)azo-5-methylbenzoate and zinc(II) 2-(4,5-diphenylimidazol-2-yl)azo-4-nitrophenolate and combinations thereof.

In certain embodiments, the color changer is chemically modified, such as by coupling to a long-chain hydrocarbon. In these embodiments, the long-chain hydrocarbon may be sufficient to convert a water soluble color changer into an oil soluble color changer. Depending on the chemical structure, the water soluble color changer may be coupled to a long-chain hydrocarbon having chain lengths of C5, C6, C7, C8, C9, C10, C11, C12, C13, C14, C15, C16, C18, C19, C20, C21, C22, C23, C24, C25, C26, C27, C28, C29, C30 and longer synthetic and/or naturally derived hydrocarbon chain lengths. In other embodiments, the color changer is chemically modified by coupling to a polyalkylene glycol, such as a polyethylene glycol or a polypropylene glycol. In these embodiments, the polymeric length of the polyalkylene glycol is sufficient to convert an oil soluble color changer into a water soluble color changer. For example, the polymeric length of the polyalkylene glycol may be 2 or more, such as 3 or more, such as 5 or more, such as 7 or more, such as 10 or more, such as 15 or more and including 20 or more.

The solubility of the color changer ranges from being greater than 99.999% insoluble in water to being greater than 99.999% soluble in water. Where a solvent is present, as described below, the color changer is soluble in the solvent, such as an aqueous soluble, a hydrocarbon solvent, a long chain hydrocarbon alcohol solvent among other types of solvents.

The amount of color changer may vary depending on the type of color changer, colored pigment and volume of the evanescent color change composition and may range from 1% to 99% w/w, such as from 5% to 95% w/w, such as from 10% to 90% w/w, such as from 15% to 85% w/w, such as from 20% to 80% w/w, such as from 25% to 75% w/w, such as from 30% to 70% w/w, such as from 35% to 65% w/w and including from 40% to 60% w/w. In certain embodiments, the molar ratio of color changer to colored pigment ranges from a molar ratio of from 0.01 to 50, such as a molar ratio from 0.05 to 45, such as a molar ratio of from 0.1 to 25, such as a molar ratio of from 0.5 to 20, such as from a molar ratio from 1 to 15 and including a molar ratio of from 1 to 10.

The intensity of the light sufficient to color transition the evanescent color change composition may vary depending on the wavelength of light and type of color changer (e.g., free radical photoinitiator, singlet oxygen sensitizer) employed and may be 10 mW/dm² or greater, such as 15 mW/dm² or greater, such as 25 mW/dm² or greater, such as 50 mW/dm² or greater, such as 100 mW/dm² or greater, such as 250 mW/dm² or greater, such as 500 mW/dm² or greater, such as 1000 mW/dm² or greater, such as 1500 mW/dm² or greater, such as 2000 mW/dm² or greater, such as 2500 mW/dm² or greater, such as 3000 mW/dm² or greater, such as 3500 mW/dm² or greater, such as 4000 mW/dm² or greater, such as 4500 mW/dm² or greater and including 5000 mW/dm² or greater.

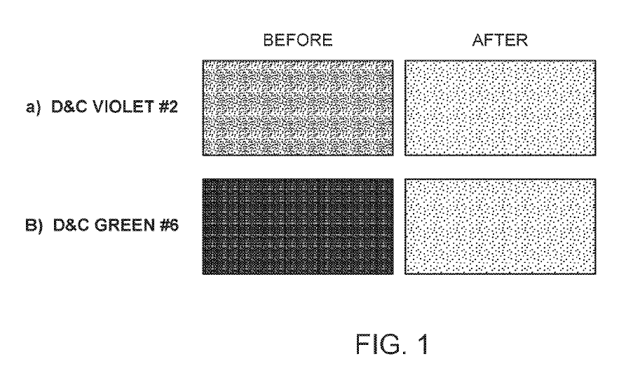

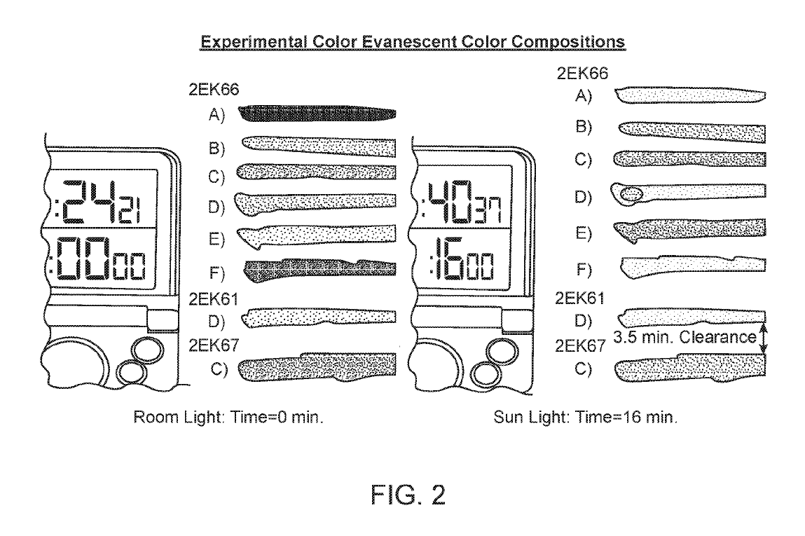

FIGS. 1a-b depict an example of 2 different evanescent color change compositions exhibiting color evanescence according to certain embodiments. FIG. 1a depicts a composition having D&C Violet #2 dye and a photoinitiator. Before application of light to the composition, the composition has a violet color. After subjecting the composition to light for a period of time, the violet color completely fades and the composition appears colorless. FIG. 2a depicts a composition having D&C Green #6 dye and a photoinitiator. Before application of light to the composition, the composition has a green color. After subjecting the composition to light for a period of time, the green color completely fades and the composition appears colorless.

FIG. 2 depicts an example of 8 different evanescent color change compositions exhibiting color evanescence according to certain embodiments. As depicted in FIG. 2, compositions having blue, yellow, pink, red, and orange color fade upon exposure of sunlight for 16 minutes. Each of the compositions loses its original color after 3.5 minutes.

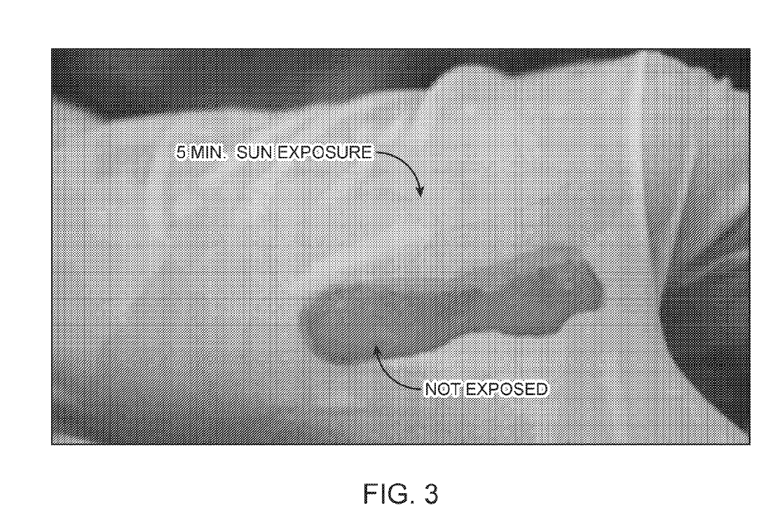

FIG. 3 depicts an example of a blue colored composition exhibiting color evanescence according to certain embodiments. As depicted in FIG. 3, the blue color fades upon exposure to sunlight for 5 minutes.

In certain embodiments, to control the reaction rate and decomposition of the colored pigment (resulting in color evanescence), the internal phase of the composition can be adjusted, modified, and balanced. By way of example, not limitation, internal phases and mediums can be adjusted or altered to increase the evanescent reaction rate, decrease the reaction rate, increase the reaction completion, decrease the reaction completion, reduce background or residual color, provide a hue alteration, refine reaction rates, develop complex reaction sequences control reaction sensitivity to light intensity, control reaction sensitivity to light bandwidth sensitivities and the like.

Internal phases can include a range of different materials and compositions that facilitate, augment, are benign, are interactive, are non-reactive, facilitate or play no role in the evanescent process other than to harbor the subject composition to react independent of the internal phase itself. Internal phases components or compositions can be oils, hydrocarbon waxes, long, medium or short chain fatty acid or alcohols, esters, amides, reactive chemistries, none-reactive chemistries, biphasic molecules, lipids, amphiphylic moieties, pure compositions, mixed compositions, organic oils, inorganic oils, silicon oils, branched or strait chained, aromatic or non-aromatic, polymeric or monomeric or the like. The internal phase of interest will depend on the evanescent components selected and the desired properties and characteristics of the resulting evanescent effect and final formulation of interest.

Not limited by mechanism, internal phase compositions that promote low residual color and facilitate fast and complete color extinction are desirable in most cases where the evanescent color system is to be incorporated into a topical cream, ointment, sunscreen or other product that would benefit by indicating colorant based uniform application.

In some embodiments, one or more components of the subject compositions are encapsulated, such as being microencapsulated or macroencapsulated. In certain embodiments, one or more of the colored pigment and color changer are encapsulated, such as microencapsulated or macroencapsulated. Additives (e.g., antioxidants, binding agents, solvents, etc.) to the subject compositions, as described in greater detail below, may also be encapsulated, as desired. The term “microencapsulated” is used in its conventional sense to refer to surrounding or enveloping one or more compounds within a capsule of another compound, yielding microcapsules having diameters that range from less than one micron to several hundred microns in size. For example, microcapsules provided by the invention may have diameters ranging from 1 μm to 1000 μm, such as 5 μm to 900 μm, such as 10 μm to 800 μm, such as 25 μm to 750 μm and including 50 μm to 500 μm. In other embodiments, capsules having one or more components of the subject compositions may have diameters that range from 1 nm to 1000 nm, 5 nm to 900 nm, such as 10 nm to 800 nm, such as 25 nm to 750 nm and including 50 nm to 500 nm. In still other embodiments, capsules having one or more components of the subject compositions may have diameters that range from 1 mm to 10 mm, such as from 2 mm to 9 mm, such as from 3 mm to 8 mm, such as from 4 mm to 7 mm and including from 5 mm to 6 mm.

Microcapsules may include a homogeneous mixture of the one or more compounds within the microcapsule or may have a plurality of distinct droplets of each compound. In some embodiments, the subject compositions are pure microcapsule compositions having only a single microcapsule composition. In other embodiments, the subject compositions may include multi-independent microcapsule compositions, where the composition is a mixture of 2 or more distinct microencapsulated compositions, such as 3 or more, such as 4 or more, such as 5 or more, such as 6 or more, such as 7 or more, such as 8 or more, such as 9 or more and including a multi-independent microcapsule composition that includes 10 or more distinct microencapsulated compositions.

Microencapsulation may be achieved by any suitable technique depending on the application of interest. Compositions may be microencapsulated with the intention that the core material be confined within capsule walls for a predetermined period of time. In some embodiments, the core material may be released immediately (i.e., substantially all at one time), such as where the capsule walls are degraded, ruptured, melted or dissolved. Alternatively, core materials may be encapsulated so that the core material will be released either gradually through the capsule walls through controlled release or diffusion, or when external conditions (e.g., heat, acid, light, etc.) trigger the capsule walls to gradually rupture, melt, or dissolve.

The core material in the subject microencapsulated compositions may include 1 or more components of the evanescent color change compositions described herein, such as 2 or more components, such as 3 or more components, such as 4 or more components, such as 5 or more components, such as 6 or more components, such as 7 or more components, such as 8 or more components, such as 9 or more components and including 10 or more components. In certain embodiments, the core material is a pure core composition and each microcapsule includes only a single component of the subject evanescent color change composition. For example, the core material may include only the color changer or the color pigment, etc.

Depending on the type of component being microencapsulated, the core materials in the subject microencapsulated compositions may be in any physical form, such as a liquid, slurry, dispersion, colloidal suspension, solid, powder, crystalline solid, flakes etc.

The core may be singly or multiply encapsulated, as desired. In some embodiments, the subject encapsulated compositions include a singly encapsulated core composition. In other embodiments, the subject encapsulated compositions include a multiply encapsulated core composition, such as a doubly encapsulated, a triply encapsulated, a quadruply encapsulated and a quintuply encapsulated core compositions.

Microencapsulation can be achieved by chemical processes and mechanical or physical processes including, but not limited to bulk fluid processes, phase separation processes, chemical processes, mechanical shear processes and milling processes. Compositions discussed herein can be microencapsulated using coacervation, interfacial polymerization, polymer-polymer incompatibility, phase separation processes, oil-in-water microencapsulation, reverse phase water-in-oil microencapsulation, environmental triggered release microencapsulation, entrapped microencapsulation, pulverized entrapped microencapsulation, natural ecologically balanced encapsulation processes, centrifugal processes, high-shear processes, mechanical drying processes, fluid bed coating, Wurster processes, centrifugal extrusion, ultrasonication/coating, rotational suspension, double wall micro-encapsulation, chemical silanization processes, liposomal encapsulation, in-line printing/layering processes, heat/chilling cycling, embedding, in-situ polymerization, urea-formaldehyde systems, melamine formaldehyde systems, formaldehyde-free microencapsulation, impregnation, particle coating, and a variety of other micro-particle formation/microencapsulation processes or the like. In certain embodiments, the subject compositions may be macro-encapsulated.

Complex coacervation may be employed to microencapsulate any of the compositions described herein. In the subject coacervation process gelatin having a high iso-electric point and gum arabic containing many carboxyl groups are added to a core-containing suspension at relatively low pH above 35° C. The gelatin and gum Arabic react to form microdroplets of polymer coacervate which separate. The wall can be subsequently hardened by several means such as by the addition of formaldehyde or glutaraldehyde. In the final steps, the suspension of microcapsules is cooled and the pH raised after which the suspension is filtered leaving the microcapsules on the filter media.

Core/shell material properties can be utilized during microencapsulation based on variety of properties including, but not limited to: gas/liquid/solid, solubility, iscosity/surface tension, density, reactivity, capsule size, capsule percent payload, capsule morphology, production capacity, release profile mechanism if any is desired, stability, encapsulation processes, shell stability, shell hardness or softness, single walled or multi-walled encapsulation, Food & Drug Administration and regulatory guidelines, robustness, staining characteristics, particle size, particle uniformity, ease of production, ability to be surface modified, selective application, phase characteristics, if micro-spheres are intended to be dried or not, if particles are intended to purified and if so the method for purification, and the like.

In embodiments, the subject composition may be microencapsulated by any convenient microencapsulation protocol, including but not limited to solvent evaporation, in situ polymerization, interfacial polymerization, emulsion polymerization, simple and complex coacervation, layer-by-layer deposition, liposomes, oil-in-water, emulsions, water-in-oil emulsions, core-shell capsules or matrix particles, stable, high-solid dispersions, chemical encapsulation, nanoencapsulation, micelles, polymersomes, phase inversion/precipitation, polyelectrolyte complexes, controlled precipitation, surfactant-free particle formation and templating, among other microencapsulation protocols.

Components for interfacial polymerization micro-encapsulation can include, but are not limited to polymelamine-co-formaldehyde, polyurea-co-formaldehyde, polyphenol-co-formaldehyde, etc, ethylene glycol dimethacrylate esters, PEG-modified capsule formulations, stain resistant microcapsule formulations, 1,6-hexanediol diacrylate, 1,4-butanediol diacrylate, aliphatic urethane acrylate, aromatic urethane acrylate, polyester acrylate oligomer, ethoxylated trimethylol propane triacrylate esters, amine modified ethoxylated trimethylol propane triacrylate, amine modified polyether acrylate oligomer and polyethylene glycol 400 diacrylated and combinations thereof. In certain embodiments, the core material includes a soybean oil material such as an epoxidized soybean oil core material.

Emulsifiers and colloid protector components for microencapsulation may include, but are not limited to, poly(methyl vinyl ether-alt-maleic anhydride), poly(vinyl alcohol), poly(styrene-alt-maleic anhydride), gum arabic from acacia tree, polyvinylpyrrolidone, hydroxyethyl cellulose, hydroxypropyl cellulose, gelatin from porcine skin, poly(acrylic acid), Span 80, Tween 20, sorbitan monostearate (same as span 60 and tween 60), glyceryl monostearate, polyethylene glycol, propylene glycol mono-laurate, glycerol mono-oleate, glyceryl stearate, sodium dodecyl sulfate among other emulsifiers as well as combinations thereof.

In some embodiments, the capsule surface may be modified. For example, the capsule may be surfaced modified with one or more surface modifying agents to include a modifying group, such as a reactive group (e.g., hydroxyl, siloxyl, sulfhydryl, amine, carboxyl, ester, nucleophile or electrophile, etc.) In some embodiments, encapsulated compositions include capsules (e.g., microcapsules, macrocapsules, etc.) 1 or more surface modifying groups, such as 2 or more surface modifying groups, such as 3 or more surface modifying groups and including 5 or more surface modifying groups. In certain embodiments, the capsule surface is modified with a polymer, such as a polyethylene glycol polymer.

In some embodiments, the capsule is formed from a fracture resistant compound, such that the capsule is resistant to fracture and premature release of the core components. In other embodiments, the capsule is formed from a shear susceptible encapsulant. The shear susceptible encapsulant may have a shear modulus of 1 pascal or more, such as 2 pascals or more, such as 5 pascals or more, such as 10 pascals or more, such as 50 pascals or more, such as 100 pascals or more, such as 1000 pascals or more, such as 10⁴ pascals or more, such as 10⁵ pascals or more, such as 10⁶ pascals or more and including a shear susceptible encapsulant having a shear modulus of 10⁹ pascals or more.

In some embodiments, by encapsulating the subject color change compositions, such as where one or more components of the color change compositions are microencapsulated, the staining ability of the composition is reduced as compared the same composition where the components are not encapsulated (e.g., microencapsulated). For example, the staining ability of the composition having one or more of the components being encapsulated (e.g., microencapsulated) is reduced by 10% or more, such as by 15% or more, such as by 25% or more, such as by 35% or more, such as by 50% or more, such as by 60% or more, such as by 70% or more, such as by 75% or more, such as by 80% or more, such as by 90% or more, such as by 95% or more, such as by 96% or more, such as by 97% or more, such as by 98% or more, such as by 99% or more, such as by 99.9% or more and including by 99.99% or more. For example, the staining ability may be reduced by 1.5-fold or more, such as 2-fold or more, such as 3-fold or more, such as 4-fold or more, such as 5-fold or more, such as 10-fold or more, such as 25-fold or more, such as 50-fold or more and including 100-fold or more. In certain embodiments, the staining ability of the composition having one or more of the components being encapsulated (e.g., microencapsulated) is reduced by 100%, such as where the composition has entirely lost its ability to stain (e.g., stain a surface). In certain embodiments, the subject evanescent color change compositions include one or more timed-release agents, such as where a component is released from encapsulation (e.g., microcapsule, macrocapsule, etc.) after a predetermined period of time. For example, the timed-release agent may be released from encapsulation in the subject compositions after a predetermined period of time after an applied stimulus. In certain instances, the predetermined duration is 5 seconds or more after the applied stimulus, such as 10 seconds or more, such as 30 seconds or more, such as 60 seconds or more, such as 5 minutes or more, such as 10 minutes or more, such as 15 minutes or more, such as 30 minutes or more, such as 60 minutes or more, such as 2 hours or more, such as 3 hours or more, such as 6 hours or more and including 12 hours or more after the applied stimulus. Any encapsulated component of the subject evanescent color change compositions described herein may be time-released, such as the color change component, colored pigment, antioxidant, solvent, etc.

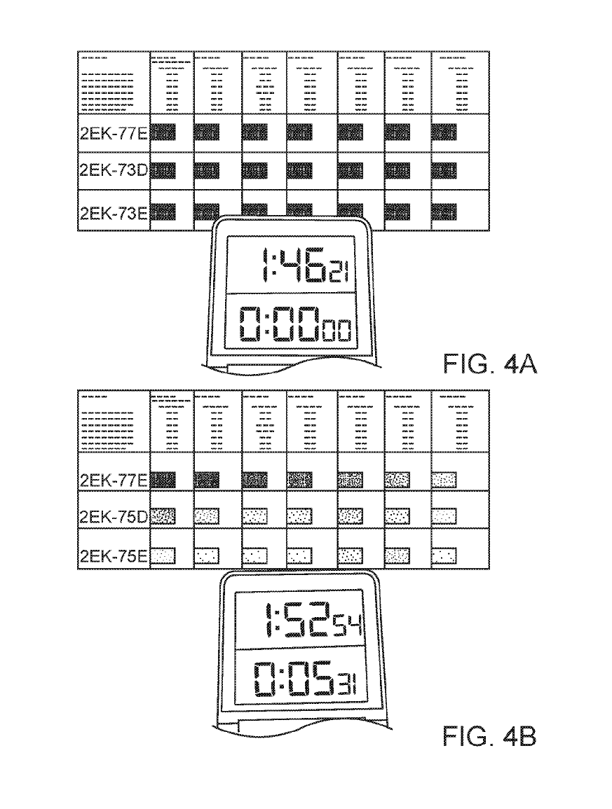

FIGS. 4a-b depict an example of 21 microencapsulated blue evanescent color change compositions incorporated into different sunscreens exhibiting color evanescence according to certain embodiments. Before exposure to sunlight, the compositions have a blue color (FIG. 4a). After exposure to sunlight for 5.5 minutes, the microencapsulated blue colored compositions begin to fade, some being completely faded (FIG. 4b). The compositions were stored for 3 weeks before exposure to sunlight and demonstrate that the subject compositions exhibit good stability for 3 or more weeks.

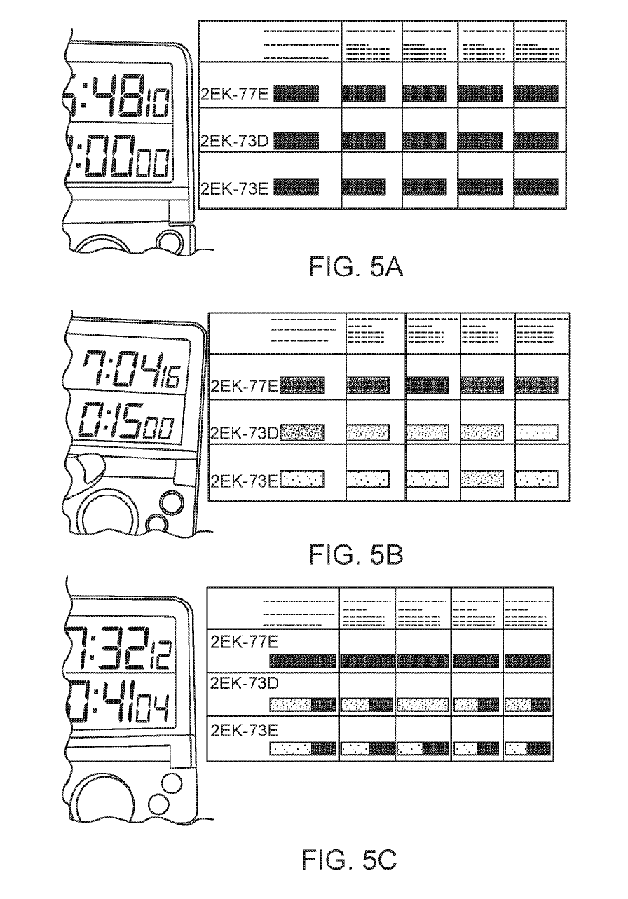

FIGS. 5a-c depict an example of microencapsulated blue evanescent color change compositions incorporated into different sunscreens and exposed to different durations of sunlight according to certain embodiments. Before exposure to sunlight (FIG. 5a), each of the compositions retains a blue color. After exposure for 15 minutes (FIG. 5b), the color of the compositions begin to fade. After exposure for an additional 26 minutes (41 minutes total), the compositions are almost completely colorless (FIG. 5c).

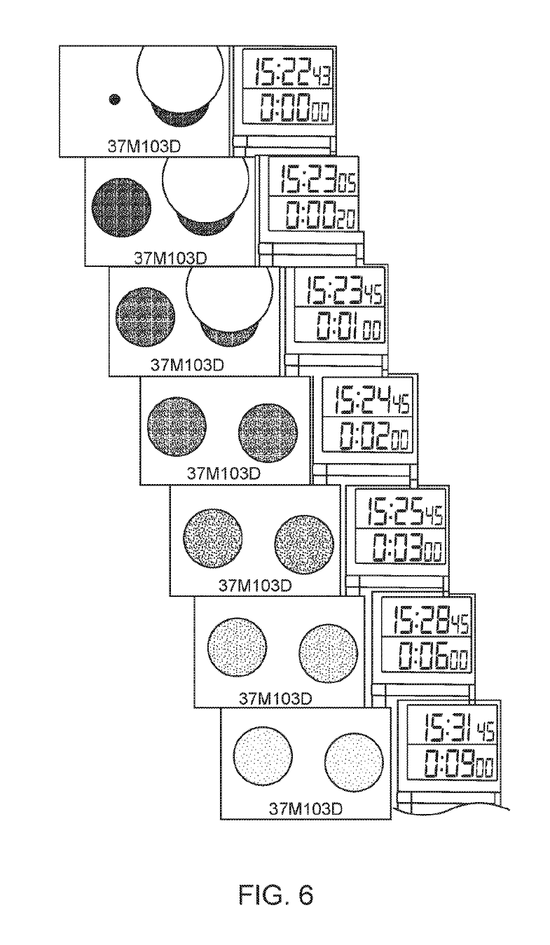

FIG. 6 depict an example of color evanescence of a microencapsulated blue evanescent color change composition over time according to certain embodiments. Before exposure to light (at time=0), the composition retains a blue color. After 1 minute, the color begins to fade until by 6 minutes, the composition is almost completely colorless. By 9 minutes the blue evanescent color change composition is no longer visible.

Alternate micro-encapsulation resins including but not limited to urea formaldehyde, melamine formaldehyde, combinations thereof, epoxy-based resins, non-formaldehyde systems, acrylic systems, isothiocyanate systems, gelatin systems, epoxy-imidazole resin systems, polyurethane systems, may be employed in some instances.

Regarding interfacial polycondensation, the two reactants in a polycondensation meet at an interface and react rapidly. The basis of this method is the classical Schotten-Baumann reaction between an acid chloride and a compound containing an active hydrogen atom, such as an amine or alcohol, polyesters, polyurea, polyurethane. Under the right conditions, thin flexible walls form rapidly at the interface. A solution of the pesticide and a diacid chloride are emulsified in water and an aqueous solution containing an amine and a polyfunctional isocyanate is added. Base is present to neutralize the acid formed during the reaction. Condensed polymer walls form instantaneously at the interface of the emulsion droplets.

Interfacial cross-linking is derived from interfacial polycondensatio. In this method, the small bifunctional monomer containing active hydrogen atoms is replaced by a biosourced polymer, like a protein. When the reaction is performed at the interface of an emulsion, the acid chloride reacts with the various functional groups of the protein, leading to the formation of a membrane. The process is versatile, and the properties of the microcapsules (size, porosity, degradability, mechanical resistance) can be customized.

In some microencapsulation embodiments, the direct polymerization of a single monomer is carried out on the particle surface. In one process, e.g., cellulose fibers are encapsulated in polyethylene while immersed in dry toluene. Usual deposition rates are about 0.5 μm/min. Coating thickness ranges 0.2-75 μm (0.0079-2.9528 mils). The coating is uniform, even over sharp projections. Protein microcapsules are biocompatible and biodegradable, and the presence of the protein backbone renders the membrane more resistant and elastic than those obtained by interfacial polycondensation.

In some embodiments, a core material is embedded in a polymeric matrix during formation of the particles. A simple method of this type is spray-drying, in which the particle is formed by evaporation of the solvent from the matrix material. The solidification of the matrix also can be caused by a chemical change.

The application of interest for evanescent color-fade compositions will in part dictate the type of resin system, the core composition, and the microencapsulation process of interest.

Both encapsulating and entrapping resins systems can comprise a pigment binder for evanescent color-fade compositions. Whereas encapsulating resin systems can form a shell around a core phase containing an evanescent formulation, an entrapping resin can be used as a solid matrix to entrap, but not necessarily encapsulate the evanescent formulation. By way of example, not limitation, entrapping resins can be plastics, polymeric resin types and the like.

Likewise, dye systems intended for evanescence can include, but are not limited to standard dyes or FD&C dyes. Dyes can include leuco-dyes, fluorescent dyes, natural dyes, liquid crystal dyes, pressure sensitive dyes, dyes used for organic light emitting diodes, organic and inorganic dyes, and the like. The application of a dye or dye system of interest will dictate the type of dye used.

Similarly, one or more photo-initiators can used in an evanescent color-fade formulation. Single or up to multiple photo-initiators provide for incremental options for wave-length activation, reaction rates, complex reactions, cost effectiveness for the formulation and the like. From one to over 20 photo-initiators types can be utilized. More often from 1 to 10 can be used. Most often, from one to up to 4 will be used for practical applications.

Evanescent color change compositions described herein may further include one or more additives. Additives may be added to modify one or more characteristics of the chromic transition (e.g., rate of color evanescence, wavelength of light to stimulate color transition) or may be added to modify a physical property of the composition (e.g., viscosity, tackiness, etc.).

In some embodiments, evanescent color change compositions include an ultraviolet (UV) light absorber. The UV absorber may be a UV-A absorber, a UV-B absorber, a UV-C absorber or a combination thereof. For example, the UV absorber may absorb electromagnetic radiation having a wavelength that ranges from 100 nm to 400 nm, such as from 125 nm to 375 nm, such as from 150 nm to 350 nm, such as from 175 nm to 325 nm and including from 200 nm to 300 nm. In certain embodiments, the UV absorber has minimum absorbance overlap with the color changer (as described above).

Examples of suitable UV absorbers may include, but are not limited to, titanium dioxide, zinc oxide, bemotrizinol, octocrylene, 2,4,6-trimethylbenzoyldiphenylphosphine oxide, p-Aminobenzoic acid (PABA), octyldimethyl-PABA, phenylbenzimidazole sulfonic acid, 2-ethoxyethyl p-methoxycinnamate, dioxybenzone, oxybenzone, homomethyl salicylate, menthyl anthranilate, 2-cyano-3,3-diphenyl acrylic acid 2-ethylhexylester, 2-ethylhexyl-paramethoxycinnamate, 2-ethylhexyl salicylate, 3-benzoyl-4-hydroxy-6-methoxybenzenesulfonic acid, triethanolamine salicylate, 1-(4-methoxyphenyl)-3-(4-tert-butylphenyl)propane-1,3-dione, terephthalylidene dicamphor sulfonic acid, 4-methylbenzylidene camphor, methylene bis-benzotriazolyl tetramethylbutylphenol, tris-biphenyl triazine, disodium phenyl dibenzimidazole tetrasulfonate, drometrizole trisiloxane, sodium dihydroxy dimethoxy disulfobenzophenone, ethylhexyl triazone, diethylamino hydroxybenzoyl hexyl benzoate, diethylhexyl butamido triazone, dimethico-diethylbenzalmalonate, isopentyl-4-methoxycinnamate or a combination or mixture thereof.

In some embodiments, the UV absorber is a hindered amine light stabilizer, where in certain instances, the hindered amine light stabilizer is substantially the same as Tinuvin® 152, Tinuvin® 328, Tinuvin® 5050, Tinuvin® 5100, Tinuvin® 5272, Tinuvin® 928, Tinuvin® 99, Tinuvin® 109 Tinuvin® 111 FDL Tinuvin® 1130 Tinuvin® 123, Tinuvin® 123-DW, Tinuvin® 144, Tinuvin® 1577 ED, Tinuvin® 1600, Tinuvin® 171, Tinuvin® 213, Tinuvin® 234, Tinuvin® 292 and Tinuvin® 292 HP.

The amount of UV absorber in the subject compositions may vary ranging from 1% to 99% w/w, such as from 5% to 95% w/w, such as from 10% to 90% w/w, such as from 15% to 85% w/w, such as from 20% to 80% w/w, such as from 25% to 75% w/w, such as from 30% to 70% w/w, such as from 35% to 65% w/w and including from 40% to 60% w/w. In certain embodiments, the molar ratio of UV absorber to color changer ranges from a molar ratio of from 0.01 to 100, such as a molar ratio from 0.05 to 95, such as a molar ratio of from 0.1 to 50, such as a molar ratio of from 0.5 to 40, such as from a molar ratio from 1 to 30 and including a molar ratio of from 1 to 10.

As described in greater detail below, in certain instances, the subject color change composition is a sunscreen composition. Sunscreen compositions of interest include a liquid dispersion having a UV absorber and one or more of the subject evanescent color change compositions described above. For example, the UV absorber may include one or more of a UV-A absorber, UV-B absorber and a UV-C absorber. In these embodiments, one or more components of the evanescent color change sunscreen compositions may be encapsulated (e.g., microencapsulated or macroencapsulated), such as the color changer, the colored pigment, the UV absorber or another component. In certain instances, an active ingredient (e.g., UV absorber) is encapsulated (e.g., micro or macro encapsulated). In other instances, an inactive ingredient (e.g., binder, filler) is encapsulated in the subject evanescent color change sunscreen composition.

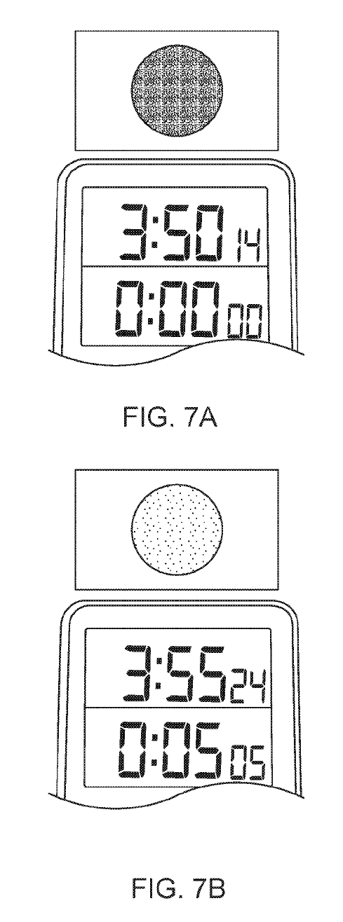

FIGS. 7a-7b depict an example of a yellow evanescent color change composition incorporated into an SPF 50 sunscreen and exposed to sunlight according to certain embodiments. Before exposure to sunlight (FIG. 7a), the composition is yellow in color. After exposure for 5 minutes (FIG. 7b), the color of the compositions is completely faded and appears colorless.

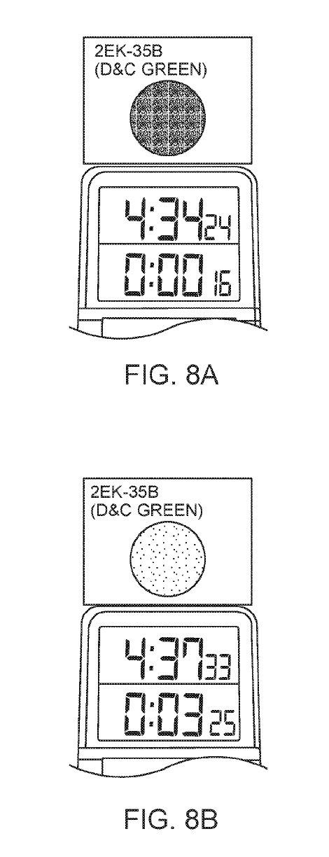

FIGS. 8a-8b depict an example of a D&C green dye evanescent color change composition incorporated into an SPF 50 sunscreen and exposed to sunlight according to certain embodiments. Before exposure to sunlight (FIG. 8a), the composition is blue-green in color. After exposure for 3.5 minutes (FIG. 8b), the color of the compositions is faded and begins to appear colorless.

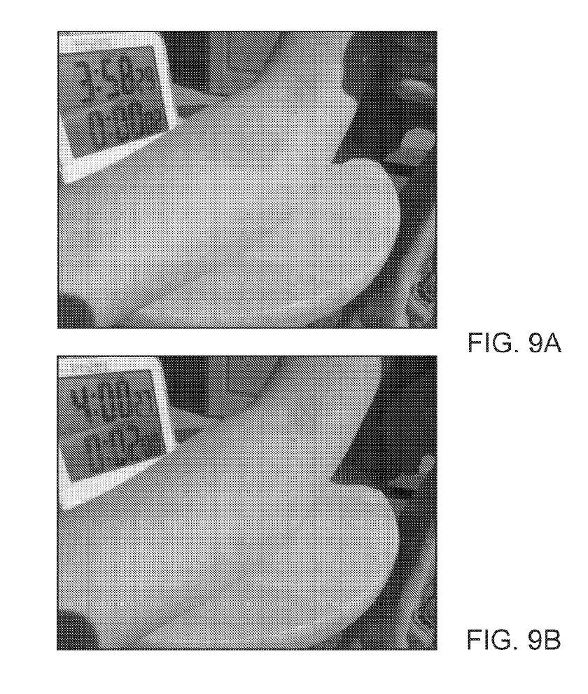

FIGS. 9a-9b depict an example of a D&C blue dye evanescent color change composition incorporated into an SPF 50 sunscreen and exposed to sunlight on the surface of the skin according to certain embodiments. Before exposure to sunlight (FIG. 9a), the composition is blue. After exposure for 2 minutes (FIG. 9b), the color of the compositions is faded and begins to appear colorless.

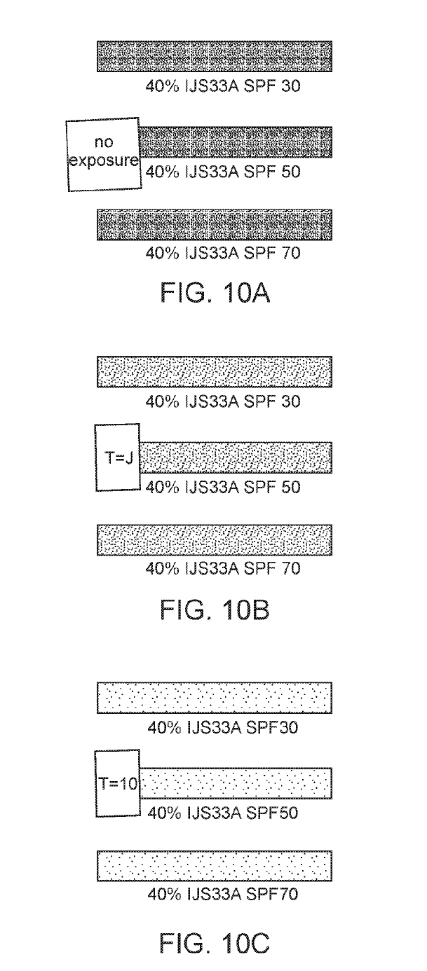

FIGS. 10a-10c depict an example of a blue evanescent color change composition incorporated into different SPF (30, 50 and 70) sunscreens and exposed to sunlight according to certain embodiments. Before exposure to sunlight (FIG. 10a), the composition is blue. After exposure for 5 minutes (FIG. 10b), the color of the compositions in each different sunscreen is faded and begins to appear colorless. After 10 minutes, all three compositions are almost completely invisible (FIG. 10c).

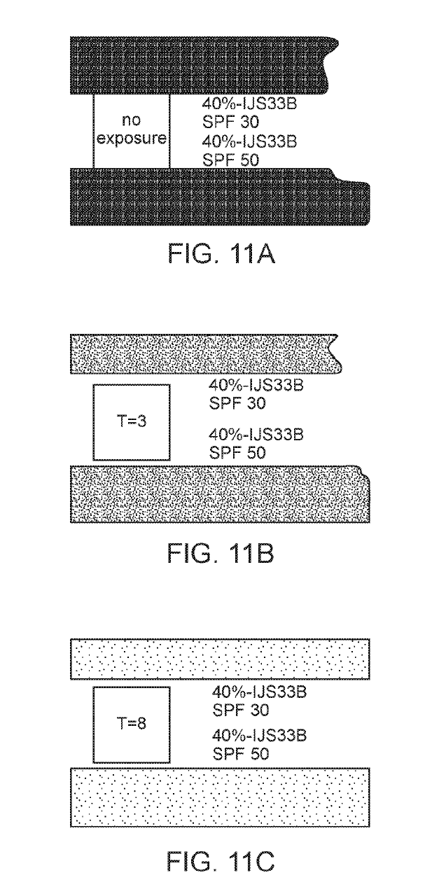

FIGS. 11a-11c depict an example of a D&C green dye evanescent color change composition incorporated into an SPF 30 and 50 sunscreens and exposed to sunlight according to certain embodiments. Before exposure to sunlight (FIG. 11a), the composition in both types of sunscreen is green. After exposure for 3 minutes (FIG. 11b), the color of the compositions is faded and begins to appear colorless. After 8 minutes, both compositions are almost completely invisible (FIG. 11c).

In some embodiments, the subject evanescent color change compositions are employed (as described in greater detail below) as neat compositions. By “neat” is meant that the color change composition includes only the components of the color change composition without any added solvents or diluents, including solid diluents, binder, fillers and the like.

In other embodiments, the evanescent color change composition further includes a solvent. Of interest are solvents that do not block the transition from colored to colorless initiated and facilitated by the color changer or alternatively provides for adjusting the wavelength of light used to catalyze the transition from colored to colorless. Non-interfering solvents of interest may include mineral oils, low temperature waxes, chloroform, methylethyl ketone, alkyl alcohols, branched or non-branched hydrocarbons such as for example n-Decane; n-Decene; n-Dodecane; n-Dodecene; n-Tetradecane; n-Tetradecene; n-Hexadecane; n-Hexadecene; n-Octadecane; n-Octadecene; n-Eicosane; n-Eicosene and parrafin blend as well as long chain hydrocarbon alcohols, such as 1-tetradecanol, 1-hexadecanol, 1-octadecanol, 1-eicosanol, 1-docosanol, oils, mineral oil, petroleum jelly, corn oil, canola oil, castor oil, and mixtures thereof.

In certain embodiments, the solvent is a purified hydrocarbon carrier, including but not limited to purified hydrocarbon carriers having chain lengths of C10, C11, C12, C13, C14, C15, C16, C18, C19, C20, C21, C22, C23, C24, C25, C26, C27, C28, C29, C30 and longer synthetic and/or naturally derived hydrocarbon chain lengths.

In certain embodiments, the solvent is a long-chain hydrocarbon alcohol. Depending on the colored pigment and color changer, long-chain hydrocarbon alcohols can include, but are not limited to alcohols of hydrocarbons having chain lengths of C10, C11, C12, C13, C14, C15, C16, C18, C19, C20, C21, C22, C23, C24, C25, C26, C27, C28, C29, C30 and longer synthetic and/or naturally derived alcohols of hydrocarbon chain lengths.

In certain embodiments, the solvent is a microcrystalline wax, paraffin base or long chain alcohol for dispersion (or as a carrier) of the color former compound, such as for example those described in U.S. patent application Ser. No. 12/643,887, the invention of which is herein incorporated by reference in its entirety.

When present, the amount of solvent in compositions of the invention may vary ranging from 0.0001% to 99% w/w, such as 0.001% to 95% w/w, such as 0.01% to 90% w/w, such as 0.1% to 85% w/w, such as 0.5% to 80% w/w, such as 1% to 75% w/w, such as 5% to 70% w/w, such as 10% to 65% w/w and including 1% to 25% w/w.

In some embodiments, the solvent is a component which promotes temperature hysteresis. Solvents which provide temperature hysteresis for compositions of the invention may include, but are not limited to stearyl 2-methylbenzoate, cetyl 4-tert-butylbenzoate, behenyl 4-cyclohexylbenzoate, myristyl 4-phenylbenzoate, lauryl 4-octylbenzoate, hexyl 3,5-dimethylbenzoate, stearyl 3-ethylbenzoate, butyl 4-benzylbenzoate, octyl 3-methyl-5-chlorobenzoate, decyl 4-isopropylbenzoate, stearyl 4-benzoylbenzoate, stearyl 1-naphthoate, cetyl phenylacetate, stearyl phenylacetate, phenyl 4-tert-butylbenzoate, 4-chlorobenzyl 2-methyl benzoate, stearyl 4-chlorobenzoate, myristyl 3-bromobenzoate, stearyl 2-chloro-4-bromobenzoate, decyl 3,4-dichlorobenzoate, octyl 2,4-dibromobenzoate, cetyl 3-nitrobenzoate, cyclohexyl 4-aminobenzoate, cyclohexylmethyl 4-amino benzoate, cetyl 4-diethyklaminobenzoate, stearyl 4-aminobenzoate, decyl 4-methoxybenzoate, cetyl 4-methoxybenzoate, stearyl 4-methoxybenzoate, octyl 4-butoxybenzoate, cetyl 4-butoxybenzoate, 4-methoxybenzyl benzoate, cetyl p-chlorophenylacetate, stearyl p-chlorophenylacetate, decyl 3-benzoylpropionate, cyclohexyl 2-benzoylpropionate, myristyl benzoate, cetyl benzoate, stearyl benzoate, 4-chlorobenzyl benzoate, benzyl cinnamate, cyclohexylmethyl cinnamate, benzyl caproate, 4-chlorobenzyl caprate, 4-methoxybenzyl myristate, 4-methoxy benzyl stearate, benzyl palmitate, 4-nitrobenzyl stearate, neopentyl caprylate, neopentyl laurate, neopentyl stearate, neopentyl behenate, cyclohexyl laurate, cyclohexyl myristate, cyclohexyl palmitate, cyclohexylmethyl stearate, 2-cyclohexyl ethyl stearate, stearyl cyclohexylpropionate, 3-phenylpropyl stearate, 4-methoxybenzyl caproate, 4-methoxybenzyl caprate, 2-chlorobenzyl myristate, 4-isopropylbenzyl stearate, phenyl 11-bromolaurate, 4-chlorophenyl 11-bromolaurate, didecyl adipate, dilauryl adipate, dimyristyl adipate, dicetyl adipate, distearyl adipate, dibenzyl sebacate, distearyl tere-phthalate, dineopentyl 4,4′-diphenyldicarboxylate, dibenzyl azodicaroboxylate, trilaurin, trimyristin, tristearin, dimyristin and distearin.

When present, the amount of solvent for promoting temperature hysteresis in compositions of the invention may vary, ranging from 0.0001% to 99% w/w, such as 0.001% to 95% w/w, such as 0.01% to 90% w/w, such as 0.1% to 85% w/w, such as 0.5% to 80% w/w, such as 1% to 75% w/w, such as 5% to 70% w/w, such as 10% to 65% w/w and including 1% to 25% w/w.

Additives may also include modifiers such as oils including organic, natural, inorganic, and synthetic oils such as corn oil, various vegetables oils, nut oils, root oils, herbal oils, paraffin oils, greases, animal fats, natural extract oils, flavor based oils, aromatic based oils, industrial oils, among other types of oil. When present, these modifiers may be present in an amount that ranges from 0.0001% to 99% w/w, such as 0.001% to 95% w/w, such as 0.01% to 90% w/w, such as 0.1% to 85% w/w, such as 0.5% to 80% w/w, such as 1% to 75% w/w, such as 5% to 70% w/w, such as 10% to 65% w/w and including 1% to 25% w/w.

In some embodiments, evanescent color change compositions may also include one or more antioxidants or preservatives. Antioxidants of interest may include, but are not limited to water soluble antioxidants, such as ascorbic acid, cysteine hydrochloride, sodium bisulfate, sodium metabisulfite, sodium sulfite among other water soluble antioxidants; oil-soluble antioxidants, such as ascorbyl palmitate, butylated hydroxyanisole (BHA), butylated hydroxytoluene (BHT), lecithin, propyl gallate, alpha-tocopherol, among other oil-soluble antioxidants; as well as metal chelating agents, such as citric acid, ethylenediamine tetraacetic acid (EDTA), sorbitol, tartaric acid, phosphoric acid, among other chelating agents.

The amount of antioxidant or preservatives in compositions of the invention may vary ranging from 0.0001% to 99% w/w, such as 0.001% to 95% w/w, such as 0.01% to 90% w/w, such as 0.1% to 85% w/w, such as 0.5% to 80% w/w, such as 1% to 75% w/w, such as 5% to 70% w/w, such as 10% to 65% w/w and including 1% to 25% w/w.

In some embodiments, the subject compositions include a time/rate controlling agent which controls the rate of the color transition exhibited by the subject compositions. In certain embodiments, the subject compositions include an accelerator to accelerate photo-decomposition of the colored pigment using an accelerator or accelerant species in addition to the photoinitiator. Accelerators may be used on a weight or molar ratio compared to a photoinitiator. Accelerators may be used from 0.01 molar ratio of a photoinitiator to greater than a 100 times in access of a photoinitiator, such as from a 0.05 molar ratio to over 10 times the molar ratio, such as from 0.1 molar equivalents to 1 molar equivalents. The final amount will depend on the intended color fade reaction rate, system stability, and overall performance of the system.

Accelerators of interest may include, but are not limited to, organic tertiary amines, such as amine acrylate derivatives including dimethylaminoethyl methacrylate and diethylaminoethyl methacrylate (DEAEMA). In self-curing compositions, the tertiary amines may include aromatic tertiary amines, such as a tertiary aromatic amine including ethyl 4-(dimethylamino)benzoate, 2-[4-(dimethylamino)phenyl] ethanol, N,N-dimethyl-p-toluidine (commonly abbreviated “DMP”), bis(hydroxyethyl)-p-toluidine, and triethanolamine.

The amount of accelerator present in the subject compositions may vary, ranging in some instances from 0.5% to 4.0% w/w, such as from 0.6% to 3.5% w/w, such as from 0.7% to 3.0% w/w, such as from 0.8% to 2.5% w/w, such as from 0.9% to 2.0% w/w and including from 1.0% to 1.5% w/w.

In certain embodiments, compositions include a modulating component positioned between the colored pigment and the color changer that is used to mitigate the color transition (e.g., from colored to colorless) at temperatures below the melting transition of the modulating component. Example of modulating components of interest include for example, films, adhesive layers, wax layers, diffusion layers, porous layers melting layers, high viscosity layers positioned between the colored pigment and color changer which is sufficient to block or delay the onset of the color to colorless transition. For instance, one or more blocking layers may be employed to provide a delay in evanescent transition after exposure to the applied stimulus for 1 second or longer, such as 5 seconds or longer, such as 10 seconds or longer, such as 30 seconds or longer, such as 60 seconds or longer and including 5 minutes or longer.

Layer thicknesses can range from 0.1 micron to over 2 millimeters, such as from 0.5 microns to 1 millimeter, such as from 1 micron to 500 microns and including from 10 to 100 microns depending on the application of interest and time delay or color development onset delay of interest for a particular product application.

Modulating agents and matrices of interest may include, but not limited to waxes, acrylics, plastic resins, carboxy methyl cellulose (CMC), printing varnishes, hydrocarbon layers, nitrocellulose, paraffin, microcrystalline waxes, natural waxes, clay coatings, coating resins, tapes, non-developer containing adhesives, particulate, micro-particulate, thin metal layers, plastic film layers, dried protein layers, dried cellulosic layers, spray coated layers, surfactant layers, emulsifiers, membranes, semi-permeable membranes, filters, transparent layers, compliant layers, sharp melting point mediums, long chain amines, long chain carboxylic acids, long chain weak acid donors, charge carrying polymers, polymerize waxes, alkylated polymers, polyenes, polyolefins, polyethylene glycols, polypropylene glycols, clay coatings, and the like.

The subject evanescent color change compositions may also include one or more buffering agents. The term “buffering agent” is used in its conventional sense to refer to a solution of weak acid (and conjugate base) or weak base (an conjugate acid) that is employed to modulate the pH of a composition. Any convenient buffering agent may be employed, including but not limited to phosphate buffers (e.g., PBS), tris-buffers, citrate buffers (e.g., sodium citrate), acetate buffers (e.g., sodium acetate) borate buffers (e.g., borax) and combinations thereof, among other buffering systems. For example, in certain instances the buffering agent is a two-component buffer of sodium phosphate and citric acid having a pH range from 3.0 to 8.0.

The amount of buffering agent in compositions of the invention may vary ranging from 0.0001% to 99% w/w, such as 0.001% to 95% w/w, such as 0.01% to 90% w/w, such as 0.1% to 85% w/w, such as 0.5% to 80% w/w, such as 1% to 75% w/w, such as 5% to 70% w/w, such as 10% to 65% w/w and including 1% to 25% w/w.

Additional colorants can also be utilized in the subject compositions. The additional colorants can be used to develop additional color effects in the system in parallel with the evanescent process. For example, the colorants can be combined within the same microcapsule containing an evanescent dye system or be added secondarily in an additive micro-capsule. Alternate colorants can include photochromic dyes and pigments, thermochromic dyes and pigments, reversible and irreversible color change dyes, polymeric dye systems such as polydiacetylenes and other conjugated color changing dye types.

Examples of additional colorants include, but are not limited to, oil soluble hydrophobic lactones, such as Specialty Grape 7 (indolyl, phenyl substituted isobenzofuranone), Specialty Green 5 (2-dibenzylamino-6-diethylaminofluoran), Specialty Magenta 16 same as Pergascript Red 1-6B (3,3-bis(octyl-2-methyl-1H-indol-3-yl)-1-3H-isobenzofuranone), Specialty Black 4, N102 (3-dimethylamino-6-methyl-7-anilinofluoran) Specialty Blue 1 same as Pergascript Blue and Crystal Violet Lactone (3,3-bis(p-dimethylaminophenyl)-6-dimethylaminophtalide), Specialty Cyan 39 (no chemical structure info in MSDS) Specialty Magenta 20 (no info in MSDS), Pergascript Green 1-2GN (phtalide compound, proprietary diaminofluoran, trade secret), Pergascript Orange 1-G (no info in MSDS), Pergascript Black 2C, or combinations thereof.

Other suitable colorants for initial color to the composition included, but are not limited to: methylene blue, amaranth, erythrocin, floxine, rose bengal, acid red, Tartrazine, Sunset Yellow FCF, Fast Green FCF, Brilliant Blue FCF, indigo carmine, phenolphthalain, sulfophthalain, Yale Violet, methyl orange, fluorescene, methyl viologene, indophenol, dimurosbetaine, bromeosin Y, laudamine B, thionine, neutral red, toluidine blue O, indocyanine green, sulfobromophthalain, uranin, lithol rubin B, lake red C, lithol red, tetrachlorotetrabrom fluorescene, brilliant lake red R, deep maroon, toluidine red, tetrabrom fluorescene, fast acid magenta, permanent red, dibromfluorescene, permanent orange, uranine, quinone yellow, WS, alizarin cyanine green F, quinizarine green SS, light green SF yellow, patent blue NA, carbathrene blue, resorcinol brown, alizarin purple SS, brilliant fast scarlet, permanent red FSR, Ponceaux SX, fast red S, oil orange SS, poral yellow 5G, fast light yellow 3G, naphthol green B, Ginea Green B, Sudan Blue B, alizarol purple, naphthol blue black, crocin, crocin blue, orange paprica, chlorophyl, cartamine, safflower yellow, beet red, direct fast yellow GC, direct fast orange, direct fast scarlet 4BS, fast red 6BLL, direct sky blue SB, direct fast turquoise blue GL, direct copper blue 2B, coprantine green G, direct fast black D, milling yellow O, acid brilliant scarlet 3R, acid violet 5B, azaline direct blue A2G, acid cyanine 6B, acid cyanine 5R, acid cyanine green G, milling brown 3G, acid fast black VLG, acid black WA, cation yellow 3G, cation golden yellow GL, cation flavin 10G, cation yellow 5GL, cation orange R, cation brown 3GL, cation pin FG, cation brilliant red 4G, cation red GTL, cation red BLH, cation red 6B, cation red SB, cation blue GLH, cation navy blue RHL, alizarine, chrome fast blue MB, chrome fast brown KE, chrome black P2B, chrome black T, fast scarlet G base, naphthol AS, naphthol AS-G, vat yellow GCN, vat orange RRTS, indigo, vat blue RSN, vat blue BC, vat brilliant green FFB, vat olive green B, vat olive T, vat brown R, vat gray M, disperse fast yellow G, disperse pink RF, disperse blue FFR, disperse blue green B, disperse yellow 5G, disperse golden yellow GG, disperse yellow RL, disperse yellow 3G, disperse orange B, disperse yellow brown 2R, disperse fast ruby 3B, disperse fast red FB, disperse red FL, disperse red GFL, disperse brilliant pink REL, disperse violet HFRL, disperse blue FB, disperse turquoise blue GL, disperse navy blue 2GL, disperse developer, fluorescent brightener WG, fluorescent brightener ERN, fluorescent brightener AT, fluorescent brighter SA, solvent orange G, solvent fast yellow 3RE, solvent fast red B, solvent fast blue HFL, reactive yellow 3G, reactive orange 2R, reactive red 3B, reactive scarlet 2G, reactive blue 3G, reactive blue R, reactive blue BR, reactive turquoise GF, reactive brilliant blue R, reactive black B, fast yellow G, fast yellow 10G, disazo yellow AAA, disazo yellow AAMX, flavane yellow, chromophthal yellow GR, methine yellow GR, methine yellow, sunset yellow lake, anthrapyrimidine yellow, isoindolinone yellow R, quinophthalone yellow, dinitroaniline orange, pyrazolone orange, dianidine orange, persian orange lake, benzimidazolone orange HL, perynone orange, pyranthrone orange, parared, naphthol red FRR, toluidine red, naphthol carmine FB, naphthol red M, naphthol red BS, naphthol red RN, pyrazolone red, permanent red 2B, lithol red, bon lake red C, lake red C, brilliant carmin 6B, brilliant carmin 3B, Bordeaux 10B, von maroon M, brilliant scarlet G, rhodamine 6G lake, mudder lake, thioindigo Bordeaux, naphthol red FGR, brilliant carmin BS, quinacridone magenta, perylene vermillian, naphthol carmin FBB, perylene red BL, chromophthal scarlet, anthrone red, naphthol red F5RK, erythrocin lake, dianthraquinolyl red, perylene red, perylene maroon, benzimidazolone carmin HF4C, perylene scarlet, amaranth lake, quinacridone red E, pyranthron red, rhodamine B lake, methyl violet lake, alizarine maroon lake, quinacridone red, dioxadine violet, thioindigo magenta, Victoria blue lake, Victoria blue 6G lake, phthalocyanine blue, alkali blue G, indanthrone blue, brilliant green lake, malachite green lake, phthalocyanine green, pigment green B, phthalocyanine green 6Y, benzimidazolone brown HFR, aniline black, dialilide yellow H10G, dialilide yellow HR, carbazole violet, metacresol purple, bromophenol blue, crystal violet, gentiana violet, bromocresol green, bromothimol blue, 3,3-bis(p-dimethylaminophenyl)phthalide, 3,3-bis(p-dimethylaminophenyl)-6-diethylaminophtalide, 3,3-bis(p-dimethylaminophenyl)-6-chlorophtalide, 3,3-bis(p-dibutylaminophenyl)phtalide, 3-cyclohexylamino-6-chlorofluoran, 3-dimethylamino-5,7-dimethylfluoran, 3-diethylamino-7-chlorofluoran, 3-diethylamino-7-methylfluoran, 3-diethylamino-7,8-benzfluoran, 3-diethylamino-6-methyl-7-chlorofluoran, 3-(N-p-tolyl-N-ethylamino)-5-methyl-7-anilinofluoran, 3-pyrrolidino-6-methyl-7-anilinofluoran, 2-[N-(3′-trifluoromethylphenyl)amino]-6-diethylaminofluoran, 2-[3,6-bis(diethylamino)-9-(o-chloroanilino)xanthyl]-benzoic acid lactam, 3-diethylamino-6-methyl-7-(m-trichloromethylanilino)fluoran, 3-diethylamino-7-(o-chloroanilino) fluoran, 3-di-n-butylamino-7-(o-chloroanilino) fluoran, 3-(N-methyl-N-n-amylamino)-6-methyl-7-anilinofluoran, 3-(N-methyl-N-cyclohexylamino)-6-methyl-7-anilinofluoran, 3-diethylamino-6-methyl-7-anilinofluoran, 3-(N,N-diethylamino)-5-methyl-7-(N,N-dibenzylamino)fluoran, benzoyl leuco methylene blue, 6′-chloro-8′-methoxy-benzoindolino-spiropyran, 6′-bromo-3′-methoxy-benzoindolino-spiropyran, 3-(2′-hydroxy-4′-dimethylaminophenyl)-3(2′-methoxy-5′-chlorophenyl)phthalide, 3-(2′-hydroxy-4′-dimethylaminophenyl)-3-(2′-methoxy-5′-nitrophenyl)phtalide 3-(2′-hydroxy-4′-diethylaminophenyl)-3(2′-methoxy-5′-methylphenyl)phtalide, 3-(2′-methoxy-4′-dimethylaminophenyl)-3-(2′-hydroxy-4′-chloro-5′-methylphenyl)phtalide, 3-(N-ethyl-N-tetrahydrofurfurylamino)-6-methyl-7-anilinofluoran, 3-(N-ethyl-N-2-ethoxypropylamino)-6-methyl-7-anilinofluoran, 3-N-methyl-N-isobutyl-6-methyl-7-anilinofluoran, 3-morphorino-7-(N-propyl-trifluoromethylanilino)fluoran, 3-pyrrolidino-7-m-trifluoromethylanilinofluoran, 3-diethylamino-5-chloro-7-(N-benzyl-trifluoromethylanilino)fluoran, 3-pyrrolidino-7-(di-p-chlorophenyl)methylaminofluoran, 3-diethylamino-5-chloro-7-(.alpha.-phenylethylamino) fluoran, 3-(N-ethyl-p-toluidino)-7-(.alpha.-phenylethylamino) fluoran, 3-diethylamino-7-(o-methoxycarbonylphenylamino)fluoran, 3-diethylamino-5-methyl-7-(.alpha.-phenylethylamino) fluoran, 3-diethylamino-7-piperidinofluoran, 2-chloro-3-(N-methyltoluidino)-7-(p-n-butylanilino)fluoran, 3-(N-methyl-N-isopropylamino)-6-methyl-7-anilinofluoran, 3-di-n-butylamino-6-methyl-7-anilinofluoran, 3,6-bis(dimethylamino)fluorenespiro(9,3′)-6′-dimethylaminophtalide, 3-(N-benzyl-N-CyClohexylamino)-5,6-benzo-7-.alpha.-naphtylamino-4′-bromofluoran, 3-diethylamino-6-chloro-7-anilinofluoran, 3-diethylamino-6-methyl-7-mesidino-4′, 5′-benzofluoran, 3-N-methyl-N-isopropyl-6-methyl-7-anilinofluoran, 3-diethylamino-6-methyl-7-(2′, 4′-dimethylanilino)fluoran and combinations thereof.

In certain embodiments, compositions of interest further include a thermochromic compound. The term “thermochromic” is used in its conventional sense to refer to a compound which changes color state in response to a change in temperature (i.e., heating or cooling). Depending on the desired color change, thermochromic compounds may change color at about −25° C. or higher, such as at about −15° C. or higher, such as at about −5° C. or higher, such as at about 0° C. or higher, such as at about 5° C. or higher, such as at about 10° C. or higher, such as at about 20° C. or higher, such as at about 25° C. or higher, such as at about 35° C. or higher, such as at about 50° C. or higher, such as at about 75° C. or higher, such as at about 90° C. or higher, such as at about 100° C. or higher, such as at about 110° C. or higher and including at about 125° C. or higher. In certain embodiments, thermochromic compounds change color between −25° C. to 125° C., such as a range between −15° C. to 115° C., such as a range between −5° C. to 105° C., such as a range between 0° C. to 100° C., such as a range between 10° C. to 90° C., such as a range between 20° C. to 80° C., such as a range between 30° C. to 70° C. and including a range between 40° C. to 60° C.

Example thermochromic dyes of interest may include but are not limited to bis(2-amino-4-oxo-6-methylpyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the photoproduct 7,7adihydro-4,7,7,7a-tetramethyl-2-phenylbenzo[b]thiophene-5,6-dicarboxylic anhydride, micro-encapsulated dyes, precise melting point compositions, infra-red dyes, spirobenzopyrans, spironnapthooxazines, spirothopyran and related compounds, leuco quinone dyes, natural leuco quinone, traditional leuco quinone, synthetic quinones, thiazine leuco dyes, acylated leuco thiazine dyes, nonacylated leuco thiazine dyes, oxazine leuco dyes, acylated oxazine dyes, nonacylated oxazine leuco dyes, catalytic dyes, combinations with dye developers, arylmethane phthalides, diarylmethane phthalides, monoarylmethane phthalides, monoheterocyclic substituted phthalides, 3-hetercyclic substituted phthalides, diarylmethylazaphthalides, bishetercyclic substituted phthalides, 3,3-bisheterocyclic substituted phthalides, 3-heterocyclic substituted azaphthalides, 3,3-bisheterocyclic substituted azaphthalides, alkenyl substituted phthalides, 3-ethylenyl phthalides, 3,3-bisethylenyl phthalides, 3-butadienyl phthalides, bridged phthalides, spirofluorene phthalides, spirobensanthracene phthalides, bisphthalides, di and triarylmethanes, diphenylmethanes, carbinol bases, pressure sensitive recording chemistries, photosensitive recording chemistries, fluoran compounds, reaction of keto acids and phenols, reactions of keto acids with 4-alkoxydiphenylamines, reactions of keto acids sith 3-alkoxdiphenylamines, reactions of 2′-aminofluorans with aralkyl halides, reaction of 3′-chlorofluorans with amines, thermally sensitive recording mediums, tetrazolium salts, tetrazolium salts from formazans, tetrazolium salts from tetazoles, and the like. Additional thermochromic compounds of interest may include, but are not limited to: light-induced metastable state in a thermochromic copper (II) complex (see e.g., Chem. Commun., 2002, (15), 1578-1579) which under goes a color change from red to purple for a thermochromic complex, [Cu(dieten)2](BF4)2 (dieten=N,N-diethylethylenediamine); bis(2-amino-4-oxo-6-methyl-pyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium)-tetrachlorocuprate(II); bis(2-amino-4-chloro-6-methylpyrimidinium) hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the photoproduct 7,7adihydro-4,7,7,7a-tetramethyl-2-phenylbenzo[b]thiophene-5,6-dicarboxylic anhydride, and the like.

In certain embodiments, compositions of interest further include a luminescent compound. For example, luminescent compounds may include visible as well as non-visible spectrum photoluminescent compounds, chemiluminescent compounds as well as solvatoluminescent and hydroluminescent compounds. Colors can be deeply enriched using fluorescent and glow-in-the-dark or photo-luminescent pigments as well as related color additives. Luminescent compounds of interest may include, but are not limited to fluorescein, fluoresceine, resourcinolphthalein, rhodamine, imidazolium cations, pyridoimidazolium cations, dinitrophenyl, tetramethylrhodamine, among other types of luminescent compounds.

The amount of luminescent agent in compositions of the invention may vary, ranging from 0.0001% to 99% w/w, such as 0.001% to 95% w/w, such as 0.01% to 90% w/w, such as 0.1% to 85% w/w, such as 0.5% to 80% w/w, such as 1% to 75% w/w, such as 5% to 70% w/w, such as 10% to 65% w/w and including 1% to 25% w/w.

In certain embodiments, the subject compositions include one or more scavengers, such as a free radical scavenger. The term “scavenger” is used herein in its conventional sense to refer to a compound which removes or deactivates an impurity or unwanted reaction product. In some embodiments, the scavenger may be a compound that enhances the visualization of the color change transition (e.g., change from colored to colorless state). In certain embodiments, the scavenger is a free radical scavenger such as a hydrazine, tocopherol, naringenin, organotin, ascorbic acid, glutathione, among other types of scavengers. In certain embodiments, the scavenger is an antioxidant, such as ascorbic acid, cysteine hydrochloride, sodium bisulfate, sodium metabisulfite, sodium sulfite among other water soluble antioxidants; oil-soluble antioxidants, such as ascorbyl palmitate, butylated hydroxyanisole (BHA), butylated hydroxytoluene (BHT), lecithin, propyl gallate, alpha-tocopherol, among other oil-soluble antioxidants; as well as metal chelating agents, such as citric acid, ethylenediamine tetraacetic acid (EDTA), sorbitol, tartaric acid, phosphoric acid, among other chelating agents.

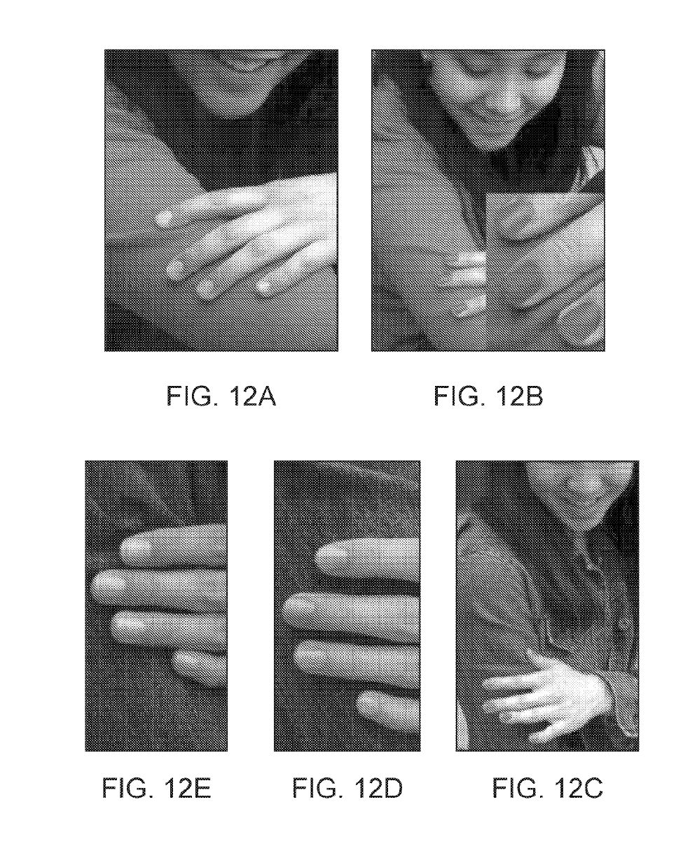

The amount of scavenger in compositions of the invention may vary, ranging from 0.0001% to 99% w/w, such as 0.001% to 95% w/w, such as 0.01% to 90% w/w, such as 0.1% to 85% w/w, such as 0.5% to 80% w/w, such as 1% to 75% w/w, such as 5% to 70% w/w, such as 10% to 65% w/w and including 1% to 25% w/w. FIGS. 12a-e depict an example of a fingernail polish composition having a photochromic dye and an evanescent color change composition according to certain embodiments. FIG. 12a depicts a subject before application of the subject fingernail polish. FIG. 12b depicts the initial pink-purple color of the fingernail polish composition. FIG. 12c depicts a color change from pink-purple to blue-purple. FIG. 12d depicts a second color change from blue-purple to light blue. After extended light exposure, the fingernail polish can be converted to back to colorless (FIG. 12e)

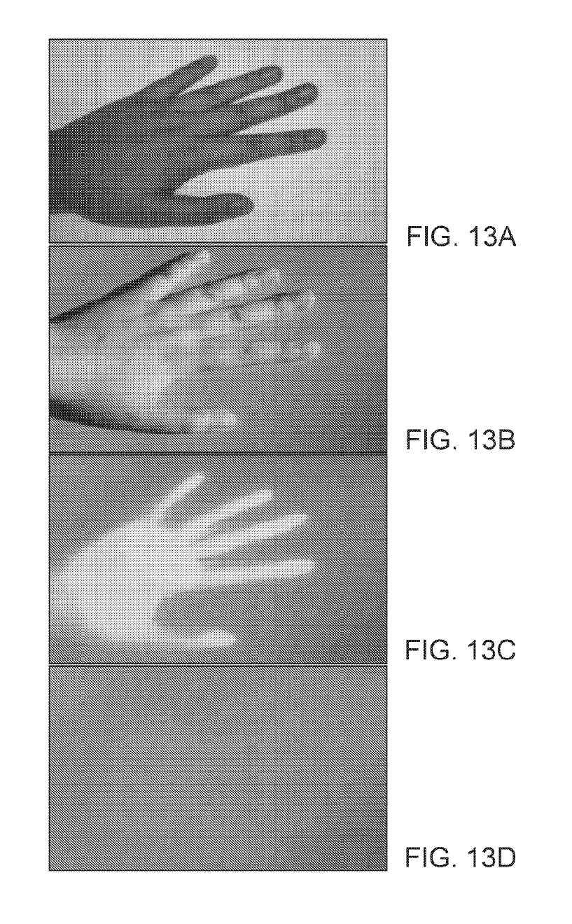

FIGS. 13a-d depict an example of a surface coating having a photochromic dye and evanescent color change composition according to certain embodiments. FIG. 13a depicts the surface before application of the coating. After application of the colored composition, the surface has a purple colored coating (FIGS. 13b-c). Exposure to sunlight results in the coating changing colors and having a uniform purple color (FIG. 13d)

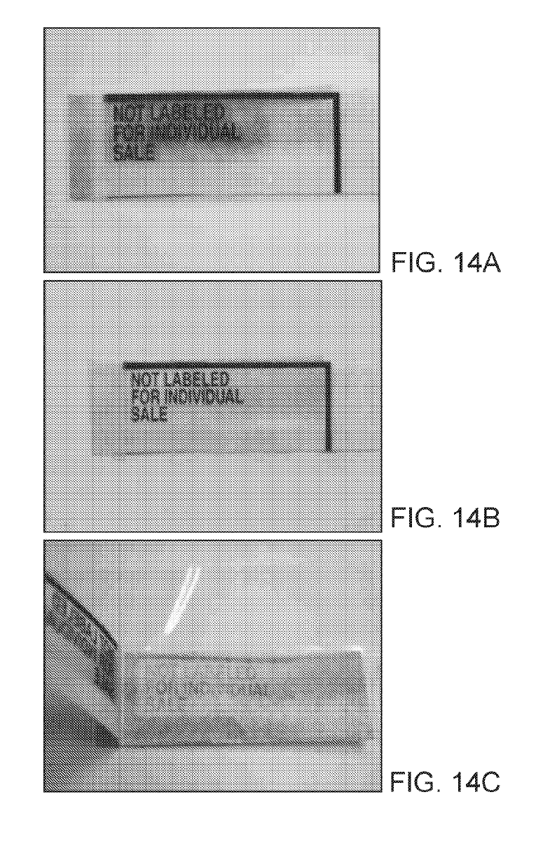

FIGS. 14a-c depict the use of an evanescent color change composition for coating a printed medium according to certain embodiments. FIG. 14a depicts coating the evanescent color change composition onto the surface of a printed label. Exposure to light results evanescence of the color (FIG. 14b) leaving behind an imprint of the evanescent color change composition where the composition was not exposed to the light (FIG. 14c).

The subject compositions may be in the (as described in greater detail below) form of a liquid, slurry, dispersion, colloidal suspension, solid, extrusion, powder, crystalline solid, flakes, etc.

### Methods for Preparing Liquid Evanescent Color Change Compositions

Aspects of the invention also include methods for preparing the color change liquid compositions having one or more of the subject evanescent color change compositions. In some embodiments, methods for preparing a color change sunscreen composition is characterized by a first process of producing an a colored pigment and color changer composition, which includes combining one or more colored pigments with one or more color changer compounds, and then a second process of producing the final color change liquid composition by combining the colored pigment and color changer composition with an amount of a solvent. Additives can be incorporated into the subject compositions either when producing the colored pigment and color changer composition or alternatively when combining the colored pigment and color changer composition with the solvent. In other embodiments the color change liquid compositions are prepared by simply combining the colored pigment, color changer compound and solvent (and additives, where desired) simultaneously. The subject compositions may be formed, as desired, into a liquid, slurry, dispersion, colloidal suspension, solid, extrusion, powder, crystalline solid, flakes, etc.

In embodiments, the liquid compositions may be formulated as a liquid coating composition. In some instances, the liquid coating composition is a personal care product, such as a nail polish, facial cream, toothpaste, body soap, hand soap, lotion, emollient, lip balm, hair care product (e.g., hairspray, gel, mousse), lipstick, cosmetic makeup or sun lotion. In certain embodiments, the liquid composition is a sunscreen. In certain instances, color change sunscreen compositions include one or more UV absorbers, such as a UV-A absorber, a UV-B absorber or a UV-C absorber.

In certain methods, an amount of one or more colored pigment and an amount of one or more color changer compounds are mixed together, either with or without a solvent, to produce a colored pigment and color changer composition. Where a solvent is not employed, one or more of the components may be in a molten state (i.e., liquid or melted) to function as a solvent. One or more additives (e.g., UV absorbers, solvent, buffering agents, thermochromic dyes, etc.) may also be mixed into the colored pigment and color changer composition. Where a solvent is employed, each additive may be separately dissolved in a solvent and then added to the colored pigment and color changer composition or the additive may be added neat into the composition.

Where the colored pigment and color changer composition is first produced, methods of the invention further include contacting an amount of the colored pigment and color changer composition with an amount of a solvent. Depending on the physical state of the colored pigment and color changer composition, the colored pigment and color changer composition may be combined with the solvent as a solid (e.g., powder, granule, flake, etc.). Where the colored pigment and color changer composition is in the form of a solid, the particle size of the colored pigment and color changer composition may be reduced before mixing with the solvent. The particle size may be reduced by any convenient protocol and may include but is not limited to lump breakers, hammermills, fine grinders, classifier mills or sifters, among other particle size reduction protocols. In certain embodiments, to reduce the particle size, the copolymer is a powder and is passed through a mesh screen. Depending on the particle size desired, the mesh screen may vary. In some embodiments, the mesh screen is a 2 mesh screen or smaller, such as a 4 mesh screen or smaller, such as a 10 mesh screen or smaller, such as a 20 mesh screen or smaller, such as a 30 mesh screen or smaller, such as a 40 mesh screen or smaller, and including a 60 mesh screen or smaller.

In certain embodiments, the liquid composition is a sunscreen composition. All of the components of the sunscreen composition (e.g., sunscreen dispersion with UV absorber, colored pigment, color changer compound and any optional additives) may be added to a mixer simultaneously. In other embodiments, each component may be added to a mixer sequentially. In certain embodiments, each component may be added to a mixer in a specific order. For example, in certain instances, methods include adding the components of the subject composition to the mixer in the order: 1) an amount of solvent, if present; 2) an amount of colored pigment; 3) an amount of color changer; 4) sunscreen dispersion with UV absorber; and 5) any desired additives. In some embodiments, each of the components may be mixed concurrently while being added to the mixer. In other embodiments, all components of the subject compositions are first added to the mixer and then the entire composition is mixed.

The subject compositions may be mixed by any convenient mixing protocol, such as but not limited to planetary mixers, Patterson-Kelley blender, hand mixers, standup mixers, inline mixers, powder liquid mixers, batch mixers, kneaders, agitator drives, impellers, hydrofoil mixers, aerators, among other mixing protocols.

In embodiments, the subject color change sunscreen compositions are mixed for an amount of time sufficient to incorporate each component and to produce a homogenous mixture. For example, the evanescent color change compositions may be mixed for 1 minute or more, such as 2 minutes or more, such as 3 minutes or more, such as 5 minutes or more, such as 10 minutes or more, and including 15 minutes or more.

In certain embodiments, the pH of the composition may be adjusted while mixing the components of the composition. By adjusting the pH is meant the pH of the composition is either increased or decreased, as desired. In some embodiments, the pH of the composition is adjusted to have a pH which ranges from 3 to 10, such as 4 to 9, such as 4.5 to 8.5 and including a pH of from 5 to 7. The pH of the composition may be adjusted using any convenient protocol. In some embodiments, the pH is decreased by adding an acid (e.g., HCI). In other embodiments, the pH is increased by adding a base (e.g., NaOH). In certain embodiments, the pH of the composition may be adjusted by adding a buffer to the any of the components, such as by adding an amount of sodium citrate dihydrate.

The properties of the subject compositions or any intermediate compositions produced during methods of preparing the subject compositions may be characterized at any convenient time. The term characterizing is used to refer to the analysis of one or more of the properties and/or components of the subject compositions or any intermediate compositions produced during methods for preparing the subject compositions. Characterizing may include, but is not limited to, determining the pH, physical properties (e.g., solid density, water content), content assay (API), spectroscopic properties, particle size distribution and impurity composition (trace metals, relating substances, etc.). Methods for analyzing compositions of the invention may include, but are not limited to the use of high performance liquid chromatography (HPLC), gas chromatography, ionization spectroscopy, among other types of analysis.

In some embodiments, methods of the invention also include assessing the properties of the characterized composition. By “assessing” is meant that a human (either alone or with the assistance of a computer, if using a computer-automated process initially set up under human direction), evaluates the determined composition and determines whether the composition is suitable or unsuitable to continue on to the next step of preparation. If after assessing that the determined composition is suitable, each composition may proceed to the following step without any further adjustments. In other words, methods of these embodiments include a step of assessing the determined composition to identify any desired adjustments.

In some embodiments, methods include monitoring each step in preparing the subject compositions. In some embodiments, monitoring includes collecting real-time data (e.g., pH, content assay, moisture content), such as by employing a detector to monitor each composition. In other embodiments, monitoring includes characterizing each composition at regular intervals, such as every 1 minute, every 5 minutes, every 10 minutes, every 30 minutes, every 60 minutes or some other interval.

In certain embodiments, methods further include purifying the subject compositions. The subject compositions may be purified by any convenient purification protocol, including but not limited to, liquid chromatography, acid-base extraction, recrystallization, filtration, among other types of purification protocols. In embodiments, the subject evanescent color change compositions are purified such that the composition includes unwanted impurities in an amount that is 5% by weight or less of the composition, such as 4% or less by weight, such as 3% by weight or less, such as 2% by weight or less, such as 1% by weight or less, such as 0.5% by weight or less, such as 0.1% by weight or less, such as 0.05% by weight or less, such as 0.01% by weight or less, such as 0.001% by weight or less and including where the composition includes impurities of 0.0001% by weight or less of the composition.

### Methods for Forming a Uniform Coating

Aspects of the invention also include methods for forming a uniform coating on a surface (e.g., mammalian skin, e.g., human skin). The term “uniform” is used herein in its conventional sense to mean that the coating has an equivalent thickness everywhere that the composition is applied. In embodiments, the thickness in the coating at any given location varies by 10% or less, such as by 8% or less, such as by 5% or less, such as by 3% or less, such as by 2% or less, such as by 1% or less, such as 0.5% or less, such as by 0.1% or less and including by 0.01% or less. In certain embodiments, the applied coating according to the subject methods is perfectly uniform and the thickness at all locations of application are identical (i.e., no variation in thickness of the coating).

Methods according to certain embodiments include applying to a surface (e.g. skin) a composition having a colored pigment and a color changer that changes the color of the colored pigment, such as from colored to colorless, in response to an applied stimulus to the color changer. By “applying” is meant contacting one or more of the compositions described above onto a surface, such as for example, onto the surface of the skin of a subject. As such, applying may include depositing or otherwise positioning one or more of the subject compositions on a surface. In certain embodiments, applying includes depositing a thin layer of the subject compositions onto a surface, such as layer having a thickness of 1 nm or more, such as 2 nm or more, such as 5 nm or more, such as 10 nm or more, such as 25 nm or more, such as 50 nm or more and including 100 nm or more. In certain instances, depositing to specific locations may include depositing the subject compositions in the form of spots (or any other geometric shape) or strips (e.g., straight or non-straight having regular and irregular patterns).

In embodiments, the subject compositions may be deposited onto any surface, as desired, where the surface may be flat, curved, smooth, rough, porous, non-porous, or any combination thereof. In certain embodiments, the surface is the surface of the skin and the subject composition is a color change skin care composition. In other instances, the subject compositions may be applied to a support, such as a wood support (e.g., floors, walls, carvings, etc.), a synthetic polymeric support (e.g., plastic sheeting, wall covers, containers, etc.), a metal support (e.g., automobile body panels, boat hulls, etc.), a fabric support (e.g., canvas, denim, etc.), a ceramic support (e.g., pottery, fiberglass, etc.), a composite support (e.g., reinforced carbon fibers, etc.) as well as combinations thereof.

The thickness of the layer of composition applied to the surface will depend on the desired properties, the rate of application, the number of layers applied and the duration of application. One or more layers of the subject compositions may be applied to the support surface. For example, two or more layers may be applied to the support surface, such as three or more layers, such as four or more layers, including 5 or more layers of the subject composition may be applied to the support surface. As described in greater detail below, additional layers of the subject compositions may be added if necessary, such as for example to improve smoothness and uniformity of the completed surface coating. For example, if after evaluating the deposited coating composition, it is determined that the composition applied to the surface of the support is less than optimal or is unsuitable, additional coating layers may be applied to all or part of the deposited coating.

The thickness of the composition applied to the surface of the support may vary depending on the coating protocol, such as 0.1 μm or more, such as 0.5 μm or more, such as 1.0 μm or more, such as 1.5 μm or more, such as 2.0 μm or more, such as μm or more, such as 10 μm or more, including 100 μm or more. For example, the composition applied to the surface may have a thickness which ranges from 0.1 μm to 250 μm, such as from 0.5 μm to 200 μm, such as from 1 μm to 150 μm, such as from 5 μm to 100 μm, such as from 10 μm to 90 μm and including from 25 μm to 75 μm. In certain embodiments, the composition applied to the surface of the support has a thickness of 50 μm.

The subject compositions may be applied by any convenient protocol, such as for example by brushing, rolling, spraying, spin coating, dip coating, mist coating, among other protocols. In certain embodiments, the composition is applied to the surface of the skin manually by hand, where a uniform coating is formed by rubbing the composition on the skin until a uniform coating is visualized. Compositions of interest may be applied to form a coating on a support surface at temperatures which vary, such as ranging from −50° C. to 250° C., such as from −25° C. to 200° C., such as from 0° C. to 150° C., such as from 10° C. to 100° C. and including from 15° C. to 85° C. In certain embodiments, the subject compositions are applied to a surface of the skin at ambient room temperature. If desired, the temperature may be modified at any time. In some instances, the temperature is not changed and remains the same throughout the entire time the composition is applied to the support surface. In other embodiments, the temperature may be increased or decreased. For example, the temperature may be increased or decreased by 0.01° C. or more, such as 0.05° C. or more, such as 0.1° C. or more, such as 0.5° C. or more, such as 1° C. or more, such as 5° C. or more, such as 10° C. or more, such as 15° C. or more, such as 20° C. or more, such as 25° C. or more, including by 50° C. or more.

After compositions are applied, methods may also include subjecting the coating to an applied stimulus, such as light. As described above, the stimulus is applied to the coating in a manner sufficient to change the color of the composition, such as from colored to colorless. The duration that the stimulus (e.g., light) is applied may vary depending on the type of colored pigment, color changer and intensity of light and may be 120 minutes or less, such as 90 minutes or less, such as 60 minutes or less, such as 30 minutes or less, such as 15 minutes or less, such as 10 minutes or less, such as 5 minutes or less, such as 4 minutes or less, such as 3 minutes or less, such as 2 minutes or less, such as 1 minute or less, such as 0.5 minutes or less and including 0.1 minutes or less.

Where the stimulus is light, the intensity of the light applied to the coating may vary depending on the wavelength of light and type of color changer employed and may be 10 mW/dm² or greater, such as 15 mW/dm² or greater, such as 25 mW/dm² or greater, such as 50 mW/dm² or greater, such as 100 mW/dm² or greater, such as 250 mW/dm² or greater, such as 500 mW/dm² or greater, such as 1000 mW/dm² or greater, such as 1500 mW/dm² or greater, such as 2000 mW/dm² or greater, such as 2500 mW/dm² or greater, such as 3000 mW/dm² or greater, such as 3500 mW/dm² or greater, such as 4000 mW/dm² or greater, such as 4500 mW/dm² or greater and including 5000 mW/dm² or greater. The color transition of the subject evanescent color change compositions may be initiated with visible light, UV-A light, UV-B light or UV-C light. For example, the color transition of the subject evanescent color change compositions may be initiated by light having a wavelength with ranges from 200 nm to 1200 nm, such as from 250 nm to 1150 nm, such as from 300 nm to 1100 nm, such as from 350 nm to 1050 nm, such as from 400 nm to 1000 nm, such as from 450 nm to 950 nm, such as from 500 nm to 900 nm and including from 550 nm to 850 nm. For example, the wavelength of light used to initiate the color transition (e.g., colored to colorless) may range from 200 nm to 390 nm, from 390 nm to 700 and including from 700 nm to 1200 nm. In certain embodiments, the wavelength of light used to initiate the color transition is 200 nm or greater, such as 225 nm or greater, such as 250 nm or greater, such as 275 nm or greater, such as 300 nm or greater, such as 325 nm or greater, such as 350 nm or greater, such as 375 nm or greater, such as 400 nm or greater, such as 425 nm or greater, such as 450 nm or greater, such as 475 nm or greater, such as 500 nm or greater, such as 550 nm or greater, such as 600 nm or greater, such as 650 nm or greater and including 700 nm or greater.

In some embodiments, more than one layer of the subject composition is applied. For example, two or more coating layers of the subject composition may be applied, such as three or more coating layers and including five or more coating layers. In some embodiments, each subsequent coating layer is applied after a predetermined period after deposition of the previous coating layer. For example, each subsequent layer may be applied, 1 hour or more after the previous coating layer is applied, such as 2 hours or more, such as 3 hours or more and including 6 hours or more after the previous coating is deposited.

Methods for forming a uniform coating on a surface may also include monitoring the composition applied. By “monitoring” is meant visualizing (with the human eye or optical detector device) the color uniformity of the applied composition as well as the color transition of the composition after applying a stimulus (e.g., light). The duration for monitoring the applied coating may vary depending on the number of layers applied, the thickness of the applied coating, as well as the type of colored pigment, color changer and intensity of stimulus applied. In some embodiments, the applied composition is monitored continuously, such as for 0.1 minutes or more, such as for 0.5 minutes or more, such as for 1 minute or more, such as for 2 minutes or more, such as for 3 minutes or more, such as for 5 minutes or more, such as for 10 minutes or more, such as for 15 minutes or more, such as from 30 minutes or more, such as for 60 minutes or more and including for 120 minutes or more. In other embodiments, the applied composition is monitored in discrete intervals, such as every 0.1 minutes, every 0.5 minutes, every 1 minute, every 5 minutes, every 10 minutes, every 30 minutes, every 60 minutes, every 100 minutes, every 200 minutes, every 500 minutes, or some other interval.

Methods of the present disclosure also include assessing the uniformity of the applied composition. By “assessing” is meant evaluating the uniformity of the applied composition to determine whether the coating is suitably uniform or whether addition layers are necessary to produce a uniform coating on the surface. For example, if after assessing that the composition applied to the surface is suitable, the desired stimulus (e.g. light) may be applied with no further adjustments to the composition.

In some instances, where the uniformity of the applied composition has been determined to be at least less than optimal, the applied composition may be modified, such as by applying additional composition to the surface or by rubbing or brushing the applied composition until it appear uniform. If necessary, the composition applied to the surface of the support may be modified one or more times, such as two or more times, such as three or more times, such as four or more times and including five or more times. For example, modifying the applied composition may include adjusting thickness. For instance, the thickness may be increased, such as by 0.1 nm or more, such as 0.5 nm or more, such as 1.0 nm or more, such as 1.5 nm or more, such as 2.0 nm or more, such as 5 nm or more, including 10 nm or more. The thickness of part or all of each coating maybe adjusted. For example, in some embodiments, methods include increasing the thickness of the entire applied coating. In other embodiments, less than that entire applied coating may be increased in thickness, such as 95% or less of the deposited layer is increased in thickness, such as 75% or less, such as 50% or less, such as 25% or less, such as 10% or less, and including 5% or less of the composition applied to the surface of the support is increased in overall thickness. In certain instances, specific regions on the composition applied to the surface of the support may be adjusted.

In other embodiments, methods include adjusting the smoothness of the applied coating. For instance, processing may include improving the smoothness of the applied coating. All or a portion of the applied coating may be processed to improve the smoothness. For example, in some embodiments, methods include improving the smoothness of the entire applied coating. In other embodiments, less than that entire applied coating may be process to improve smoothness, such as 95% or less, such as 75% or less, such as 50% or less, such as 25% or less, such as 10% or less, and including 5% or less of the composition applied to the surface of the support is processed to improve smoothness. In some instances, specific positions on the applied coating may be targeted for improving smoothness.

### Utility

The above described compositions and methods find use in any application where forming a uniform coating is desired and where an applied stimulus (e.g., light) indicator is desired.

In some embodiments, compositions of the invention find use in a variety of applications, including but not limited to early stage production, manufacturing, or synthesis stages through to end-of-use indication where a product or good being monitored using an indicator or composition has already expired and is no longer of any further utility or value.

In some embodiments, the subject compositions find use in skin care products where uniform application of the skin care product is desired but is no longer visible after application. Non-limiting examples of skin care compositions of interest may include sunscreens, sun tan lotions, facial and body makeup, lotions, baby oils, masking creams, among other types of skin care compositions.

Sunscreen lotions prevent from sunburn and reduce the risk of certain skin cancers and sun-related skin aging. Sunscreen lotions can be used for prolonged hours depending on season and exposure to sun and used by babies to adults. Non-prescription (OTC) sunscreen lotions as well as all cosmetics and skincare products with broad spectrum SPF protection are considered as a combination of cosmetics and topical drug for chronic use.

Color block technology in sunscreen lotion and Microencapsulation technique use of melamine-formaldehyde resin as a robust capsule material. Suitable commercially available shell materials for microencapsulation include Melamine-formaldehyde resin, Urea-formaldehyde/Polyoxymethylene urea resin. Alternatively, one may employ polyamides and/gelatin. Polyoxymethylene urea (PMU) is used as a bulking agent and capsule/shell material in microencapsulation technique for cosmetics and approved as a safe ingredient by cosmetic ingredient review experts. It comprises of urea and formaldehyde monomer by condensation polymerization technique. Molecular weight of the polymer varies by degree of polymerization and reaction conditions. During polymerization, it generates very low amount of formaldehyde (less than 30 ppm, less than 0.2% free formaldehyde is accepted in cosmetics application). It does not pose any serious skin irritation and toxicology effect. After curing, PMU cross-linked structure provides an excellent protection of core materials from the outside hazardous effects and superior strength, stability of the wall of the microcapsules.

The technology is not only limited by color block/evanescence applications, and can be applicable to different performance additives (fragrance, color, additives) for personal care products as well as other fields.

The subject compositions also find use in dental materials where color can be used to visualize uniform and proper implantation but is no longer visible after the dental procedure is complete. Dental materials of interest may include, but are not limited to, dental filling materials, dental sealants, glossy layers on crowns, bridges and veneers as well as clear coating layers for dental polish.

Evanescent color fade systems can be utilized in for a wide range of other applications including but not limited to: laser marking and laser induced fading in evanescent dye systems, food contact and non-food contact applications, medical and non-medical applications, writing and production printing applications, specialty inks and other encoding systems, multi-element evanescence applications where color fade can be combined with other technical optical color change effects, plastics applications and time indication means upon exposure, process monitoring in particular where light exposure is utilized in the process, signs and promotional applications, advertising applications, freshness indicating applications, security applications, forensic applications and the like. In some embodiments, the subject evanescent color change compositions may be included in inks, security inks, security compositions, anti-counterfitting compositions, plastics, coatings, pharmaceutical products, foods and beverages, promotional materials, cosmetic make-up, hand sanitizers, liquid bandages, arts and crafts, commercial signs, evanescent bill boards and signs, automotive waxes, food service sterilization, UV sterilization indication, commercial and craft paints, adhesives and glues, cleaning agents, industrial coatings, medicinal topical products, lip balms, cloth applied emollients, hair care products, hair removal emollients, polishing agents, finger nail polishes, regenerative skin emollient compositions, insect repellant, pain relief dental, dental care products such as tooth paste, printed books, magazines and newspapers, printed fliers, optical evanescent receipts, game pieces, secret messages for advertising, military and defense applications including exposure, toxic waste indication, water contamination and purification indication, radiation exposure indicators, house-hold cleaning and sanitation, free-radical induced chemo-therapeutic and immune stimulating adjuvant compositions, topical acne medications, enhanced and/or accelerated bio-degradable additives, indicators for improved safety in tanning salons and combination therapeutic agents.

The following examples are offered by way of illustration and not by way of limitation.

### EXPERIMENTAL

Example 1: Evanescent micro-capsules type—colored with no UV, fast fading under UV exposure.

Mix 0.06 g Solvent Blue 97 (from Sensient, or Beta carotene, Paprika oleoresin, etc from Color Makers or Kalsec) with 3.34 g long chain alcohol (hexadecanol, octadecanol, other) in a flask and heat with shaking in front of hot air gun until all components dissolved and liquid is not cloudy. Transfer this flask into a dark place. Add 0.6 g of UV initiator Irgacure 2100 (BASF corp.), close the lid and vortex with heating for 30 seconds. Avoid light exposure at all times from now on.

Pour the mixture into flask containing 10 g of 5% methyl vinyl ether maleic anhydride copolymer (MVEMAC) water solution, preheated on oil bath to 60 C for 2 hours or until solution became transparent. Homogenize for 2 mins with rotorstator at increasing speed from 1 to 3. Add 1 g (˜32 drops) of 5% solution of NaOH in water dropwise to mixture and keep homogenizing for 1 more minute.

Add 6 g Melamine-co-Formaldehyde 84% dissolved in butanol, preheated with hot air gun to 65 C. After 1 more minute of homogenization at speed 3 reduce the speed to 2 and add 1 g (32 drops) of 5% water solution of Acetic Acid dropwise slowly during 2 minutes. Then reduce the speed to 1 and add 1 g more (32 drops) of 5% water solution of Acetic Acid dropwise slowly during next 2 minutes. Remove rotorstator, turn off the heat and let the mixture slowly cool in oil bath overnight.

Mix at 50% with DI water in vials and centrifuge for 2 hours. Remove liquid from top, add more DI water, gently mix with wooden stick and vortex until no sediment left. Then centrifuge again for 1 hour. Repeat cleaning cycles 3 times. Remove liquid part on top and use slurry for mixing at 30% in sunscreen.
Example 2: Evanescent micro-encapsulation slurry composition.

### Materials:

Poly(methyl vinyl ether-alt-maleic anhydride) [MVEMA]
oil phase medium
oil soluble dye
photo-initiator
Poly(melamine-co-formaldehyde)
5% sodium hydroxide solution in dH2O
5% acetic acid solution in dH2O
hot oil bath filled with Crystal Plus Mineral Oil T500 (or other mineral oil)
125 ml Erlenmeyer glass flask and magnetic stir bar
hot air gun and vortex
short amber scintillation vial and two 3 oz spice jars
Omni GLH-115 Homogenizer and 30 mm rotor stator probe
parafilm, spatula, disposable 1 ml plastic pipettes

### Procedure:

1. At least two hours before starting microencapsulation, prepare a 10% solution of MVEMA in dH2O. In the 125 ml Erlenmeyer flask, add 9 g of MVEMA to 90 ml of dH2O. Add a magnetic stir bar to the vial and cover the end with parafilm. Heat the flask in the oil bath at 60° C. and stir speed 300 for at least two hours until solution becomes completely transparent, though a slight yellowish hue is fine. Store solution in the refrigerator when not in use.
2. To start the reaction, preheat the oil bath to 75° C. with a stir speed of 300.
3. Measure 20.8 g of 10% MVEMA into a 3 oz spice jar and place into the oil bath at an angle. Allow the solution to equilibrate, about 30 minutes. Leave the cap on loosely so the water in the solution does not evaporate out.
4. Add melamine formaldehyde to the second spice jar until it is half-full. Place the jar into the oil bath to warm.
5. In the amber vial, add 6.8 g of oil phase medium and 0.12 g of dye. Heat with the hot air gun and vortex until dye is dissolved and solution is transparent. Let the mixture cool and harden.
6. To the amber vial, add 1.2 g of photocompound using a spatula. Move into the dark room and turn on the red light. Heat the amber vial with a hot air gun and vortex until all components are dissolved and the solution is transparent. Add the heated solution to the jar containing the MVEMA solution.
7. Warm the rotor stator probe with a hot air gun, screw it into the homogenizer, place the probe towards the bottom of the spice jar, and run the homogenizer at speed 1. Move the rotor stator up and down to ensure homogenous mixing. After a minute, increase the speed to 2, then after another minute up to speed 3.
8. Add 2 g (64 drops) of 5% NaOH drop-wise, 8 drops at a time. Run the reaction for 5 minutes.
9. Using a pipette, add 1 mL of warm melamine formaldehyde to the reaction vessel, moving the probe up and down. Repeat ten more times until 11 mL of melamine formaldehyde is added (˜6.2 g). Run the reaction for 15 minutes.
10. Gradually add 2 g (64 drops) of acetic acid dropwise, four drops at a time, moving the probe up and down. Reduce the rotor stator speed to 2. After 5 minutes, add another 2 g (64 drops) dropwise, eight drops at a time, moving the probe up and down. Reduce the rotor stator speed to 1. Run the reaction for another 5 minutes.
11. Add hot water (˜75° C.) to the vial until it is full (about 10 ml). Turn off the heat.
12. After 15 minutes, shut off and remove the rotor stator. Allow the reaction to slowly cool in the oil bath.
13. Take a sample to test for fading. Apply a line of capsules on a sheet of white paper. Cover half the sample with aluminum foil to act as a control. Take outside in the sun and observe fading.
14. To purify capsules, centrifuge the spice jars for two five-minute intervals (about the time it takes to get to 4000 rpm). Remove the supernatant and add more dH2O. Total centrifuge time is 50 minutes (5 sets of intervals).
Example 3: Evanescent micro-encapsulation slurry composition general procedure:

### Materials:

Poly(methyl vinyl ether-alt-maleic anhydride) [MVEMA]
oil phase medium
oil soluble dye
photocompound
Poly(melamine-co-formaldehyde)
5% sodium hydroxide solution in dH2O
5% acetic acid solution in dH2O

### Apparatus:

Silverson L5M-A
OST 20 (IKA) Digital mixers
Hotplate and waterbath
Two 4 Oz spice jars, one 32 Oz glass spice jar
250 ml Erlenmeyer glass flask and magnetic stir bar
short amber scintillation vial and two 3 oz spice jars
Al foil, parafilm, spatula, disposable 1 ml plastic pipettes
Thermometers

### Procedure:

1. Prepare a 10% solution of MVEMA in dH2O. In the 250 ml Erlenmeyer flask, add 18 g of MVEMA to 180 ml of dH2O, preferably previous day. Add a magnetic stir bar to the flask and cover the end with parafilm/lid. Heat the flask in the waterbath at 60° C. and stir speed 300 for at least two hours until solution becomes completely transparent, though a slight yellowish hue is fine. Store solution in the refrigerator when not in use.
2. To start the reaction, preheat the waterbath bath to 75° C. (monitored by thermometer).
3. Take the 32 Oz spice jar and wrap with Al foil. Measure 104 g of 10% MVEMA into a 32 oz spice jar and place into the water bath. Allow the solution to equilibrate, about 30 minutes. Leave the cap on loosely so the water in the solution does not evaporate out.
4. Add 29.5 g of melamine formaldehyde to the 4 oz spice jar and make 50% dilution with DI water.
5. Internal phase (IP): Add 40 g of oil phase medium and 0.6 g of dye in the another 4 Oz jar/amber bottle and stir it with a magnetic bar for 20 minutes. Then, add 6 g of photocompound using a spatula. Move into the dark room and turn on the red light. The solution is made warm using hot waterbath while stirring continuously before adding to the reaction mixture.
6. Add the IP solution to the jar containing the MVEMA solution kept inside the waterbath.
7. Place the Silverson mixer probe (suitable for microemulsion) towards the bottom of the reaction vessel, and run at a speed of 10000 rpm for 15 minutes. Make sure, the probehead is immersed inside the solution throughout the process.
8. Add 10 g of 5% NaOH drop-wise to the reaction jar. Run the reaction for 15 minutes.
9. Using a pipette, add melamine formaldehyde solution with a pipette to the reaction vessel slowly for 30 minutes. Gradually slow down the Silverson Mixer and finally turn off.
10. Substitute the Silverson head with OST 20 (IKA) Digital mixer quickly and stir the reaction vessel gently with the paddle probe for 5 minutes.
11. Gradually add 10 g of 5% acetic acid dropwise for 5 minutes and run the reaction for 30 minutes.
12. After 30 minutes, add another 10 g 5% acetic acid dropwise. Add water (˜50 ml) slowly whenever needed [if the reaction becomes too viscous]. Run the reaction for another 3 hours.
13. Turn off the heat and allow the reaction to slowly cool in the water bath while stirring continuously until it reaches the room temperature.

### Testing:

14. Take a sample to test for fading. Apply a line of capsules on a sheet of white paper. Cover half the sample with aluminum foil to act as a control. Take outside in the sun (as well as under UV-A light) and observe fading.
15. Perform the fading test with sunscreen lotion by 10% loading of the slurry in a silimar manner as above.
16. Check the stability of the slurry inside the sunscreen lotion in a timely manner.
Example 4: Thermochromic micro-capsules type—colored at low temperature and color absence at elevated temperatures.

Mix 3 g of long chain alcohol (hexadecanol, octadecanol, other) with 0.24 g of color former: crystal violet (or Emerald Hilton Davis powder dye or Pergascript Ciba Geigy powder dye) and 0.48 g of color developer bisphenol AP (or Bisphenol A, propyl gallate, oxalic acid, etc) in a flask and heat with shaking in front of hot air gun until all components are dissolved and liquid is transparent. Pour into flask containing 10 ml water with 0.5 g MVEMAC preheated on oil bath to 60 C for 2 hours or until solution became transparent. Add 1 g (˜32 drops) of 5% solution of NaOH in water dropwise.

Add 3 g Melamine-co-Formaldehyde 84% dissolved in butanol (or Trimethylolmelamine prepared from Melamine:Formaldehyde=6.3:12=1.4 for 2 h 60 C, or Hexamethylolmelamine prepared from Melamine:Formaldehyde=3.1:12=0.7 for 2 h 60 C at pH=8-9{check if needed?}) preheated via hot air gun to 65 C. Homogenize for 15 mins with rotorstator.

Slowly dropwise add 1 g (32 drops) Acetic Acid while mixing with rotorstator, repeat 3 times every 15 mins. After 1 hour remove rotorstator and proceed mixing with magnetic stirrer for 1 hour. Let cool for 2 hours. Mix half/half with DI water in vials and centrifuge for 1 hour. Remove liquid from top, add more DI water, mix with wooden stick and centrifuge for 1 hour. Repeat cleaning procedure 3 times. Remove liquid part on top.

Mix slurry at 35% with emulsion (HDPE cationic or nonionic are preferred) using wooden stick and vortex. Dry the mixture into powder at 100 C with milling. Use for extrusion in polyethylene or polypropylene at 7%.
Example 5: Thermochromic micro-capsules type—colored at high temperatures and uncolored at low temperatures.

Mix an oil phase consisting of 1.25 g of Magenta 16 powder dye (or any Emerald Hilton Davis powder dye, or any Pergascript Ciba Geigy powder dye, or crystal violet from Sigma Aldrich, etc) with 6.25 g Glycerol Mono Stearate in a flask. Heat an oil phase with shaking in front of hot air gun until all components are dissolved and melted mixture is transparent. Dissolve 1.25 g gelatin (from Sigma Aldrich) in 12.5 ml water with 0.6 ml 5N Na₂CO₃ on oil bath at 70° C. with magnetic stirrer for 2 hours or until transparent solution obtained.

Pour an oil phase into gelatin solution. Homogenize with rotorstator for 2-5 minutes on speed 3, then reduce the speed to 2. Slowly and with vigorous stirring add 1 ml of formaline (37% solution of formaldehyde in water) dissolved in 12.5 ml of water and preheated to 70° C. Add extra 6.25 ml of water preheated to 70° C. Reduce the rotorstator speed to 1 and keep homogenizing for 20 more minutes. Let cool to room temperature.

Mix with DI water at 50% in vials and centrifuge for 1 hour. Remove liquid from top, add more DI water, mix with wooden stick and centrifuge for 1 hour. Repeat cleaning procedure 3 times. Remove liquid part on top. Mix slurry with desired medium.
Example 6: Thermocromic micro-capsules type—colored at low temperature and uncolored at high temperature.

Water phase: Mix 0.65 g PVA 97-98% hydrolyzed with 10 ml DI water and mix with magnetic stirrer at 60 C oil bath for 2 hours until dissolved and solution is transparent. Add 0.07 g initiator 4,4′azobis(4cyanovaleric acid) (from Sigma Aldrich). Homogenize with magnetic stirrer until transparent. Add 0.34 g NaOH 5% solution.

Oil phase: Mix 2.09 g of cetyl alcohol (or octadecanol, tetradecanol, lauryl alcohol, else fatty alcohols available from Sigma Aldrich and Unilin) with 0.21 g color former powder dye (from Emerald Hilton Davis, or Pergascript Ciba Geigy, or crystal violet from Sigma Aldrich, etc) and 0.21 g color developer Bisphenol AP (or Bisphenol A, propyl gallate, oxalic acid, etc, from Sigma Aldrich). Heat an oil phase with shaking in front of hot air gun until all components are dissolved and melted mixture is transparent.

Add 0.25 g of Amine modified polyether acrylate oligomer (CN551 from Sartomer), and 0.58 g of Ethylene glycol dimethacrylate (SR206 from Sartomer) to the melted oil phase and vortex. Add initiators: 0.08 g of 2,2′azobis(2methyl propionitrile), 0.04 g 1,1′azobis(cyclohexanecarbonitrile). Heat and mix well with vortex, but avoid overheating that triggers free radical formation from initiators at T above 75 C and early polymerization.

Pour oil phase into 60 C water phase (adjust T to be above Tm for each of fatty alcohols used) while on oil bath. Homogenize with rotorstator at speed 3 for 5 mins and increase the T. Proceed with homogenizing for 1 hour at T=75 C and speed 2, then for 1 hour at T=80 C and speed 1, then for 1 hour at T=90 and speed 1. Stop homogenizing and let slowly cool with oil bath.

Mix with DI water at 50% in vials and centrifuge for 1 hour. Remove liquid from top, add more DI water, mix with wooden stick and centrifuge for 1 hour. Repeat cleaning procedure 3 times. Remove liquid part on top. Store microcapsules as a slurry with a closed lid.
Example 7: Evanescent microcapsules type—colored with no UV, fast fading under UV/sun exposure.

Water phase: Mix 0.65 g PVA 97-98% hydrolyzed with 10 ml DI water and mix with magnetic stirrer at 60 C oil bath for 2 hours until dissolved and solution is transparent. Add 0.23 g 4,4′azobis(4cyanovaleric acid) (from Sigma Aldrich). Homogenize with magnetic stirrer until transparent. Add 0.34 g NaOH 5% solution.

Oil phase: Mix 3.34 g of hexadecanol (or octadecanol, tetradecanol, decyl alcohol, else fatty alcohols available from Sigma Aldrich and Unilin) with 0.06 g Solvent Blue 97 powder dye (Sensient). Heat an oil phase with shaking in front of hot air gun until all components are dissolved and melted mixture is transparent. Transfer this flask into a dark place. Add 0.41 g of UV initiator Irgacure 2100 (BASF corp.), close the lid and vortex with heating for 30 seconds. Avoid light exposure at all times from now on. Add 0.45 g of Amine modified polyether acrylate oligomer (CN551 from Sartomer), and 0.82 g of Ethylene glycol dimethacrylate (SR206 from Sartomer) to the melted oil phase and vortex.

Pour oil phase into 60 C water phase (adjust T to be above Tm for each of fatty alcohols used) while on oil bath. Homogenize with rotorstator at speed 3 for 2 mins and increase the T to 80 C without stopping rotorstator. Proceed with homogenizing at speed 2 for 2 minutes, then increase T to 90 C and reduce speed to 1. Stop homogenizing when T=90 C is reached and let slowly cool with oil bath.

Mix with DI water at 50% in vials and centrifuge for 1 hour. Remove liquid from top, add more DI water, mix with wooden stick and centrifuge for 1 hour. Repeat cleaning procedure 3 times. Remove liquid part on top. Use microcapsules as desired. Store in closed container as a slurry.
Example 8: Water in oil FD&C colored microcapsules.

Water phase: Dissolve 0.1 g of 2% solution of FD&C Blue N2 in 3.2 ml of water. Add 0.14 g (5 drops) of NaOH 5% solution in water.

Oil phase: Preheat 17 g toluene with 1.1 g of emulsifier Span 60 in a flask on oil bath to 60 C. Add 0.165 g of Amine modified polyether acrylate oligomer (CN551 from Sartomer) and 0.385 g of Ethylene glycol dimethacrylate (SR206 from Sartomer) and mix with magnetic stirrer or vortex. Add 0.08 g of 2,2′azobis(2methyl propionitrile) and keep mixing until transparent.

Remove magnetic stirrer, add oil phase and homogenize with rotorstator at speed 3 for 5 mins. Increase the T to 75 C and proceed with homogenizing for 1 hour on speed 2, then for 1 hour at T=80 C and speed 1, then for 1 hour at T=90 and speed 1. Stop homogenizing and let slowly cool with oil bath.
Example 9: Photochromic dye—uncolored with no UV and colored under UV exposure.

Mix 0.15 g photochromic dye (Photopia) with 3 g Mineral oil 500T, (or Mineral oil 70T, corn oil, canola oil, castor oil, petroleum jelly) in a flask and heat with shaking on hot air gun until all components are dissolved and liquid is transparent. Pour into flask containing 10 ml water with 0.5 g MVEMAC preheated on oil bath to 60 C for 2 hours or until transparent. Add 1 g (˜32 drops) of 5% solution of NaOH in water dropwise. Add 5 g{need to check which recipes to choose best one} Melamine-co-Formaldehyde 84% dissolved in butanol and preheated with hot air gun to 65 C. Homogenize for 15 mins with rotorstator. Slowly dropwise add 1 g (32 drops) Acetic Acid while mixing with rotorstator at slow speed, repeat 3 times every 15 mins. Add 5 ml preheated water, mix and then remove rotorstator. Let cool.

Mix half/half with de-ionized water in vials and centrifuge for 1 hour. Remove liquid from top, add more de-ionized water, mix with wooden stick and centrifuge for 1 hour. Repeat cleaning procedure 3 times. Remove liquid part on top. Store clean microcapsules in a closed container as a slurry.
Example 10: Procedure for use of Blue Evanescent Dye slurry in sunscreen types.

Store a pre-formulated evanescent dye formulation in a dark room to avoid early initiation of UV sensitive microcapsules. Shake or vortex the bottle with closed lid for 1-2 minutes to ensure homogeneity of slurry. Open the lid and use a clean stick to check that there remains no sediment of microcapsules. Use a pipette to transfer slurry into a dish intended for mixing it at 30% with sunscreen. Future formulations will be more concentrated so less Evanescent dye will be used. Mix slurry with sunscreen using stick, vortex the mixture with closed lid and store in dark to avoid light exposure until ready for use. Apply desired amount of the mixture on the 25 cm² of surface to achieve application ratio 2 mg/cm² with circular rubbing motion.
Example 11: Evanescent color fade formulation using natural dyes.
Example 12: Evanescent color fade sunscreen using natural dyes.
Example 13: Evanescent color fade formulation using co-acervation micro-encapsulation.
Example 14: Evanescent color fade formulation using gelatin-based micro-encapsulation.
Example 16: Evanescent color fade formulation using micro-encapsulation for liquid crystals.
Example 17: Evanescent color fade fluorescent dye application.
Example 18: Evanescent color fade in continuous spray sunscreens.
Example 19: Evanescent color fade in hand lotions.
Example 20: Evanescent color fade in insect repellent.
Example 21: Evanescent color fade used in building materials.
Example 22: Assays for determining evanescent color fade properties
Example 23: Titanium dioxide—hydrogen peroxide evanescence color fade system
Example 24: Internal phase adjustment for performance alteration
Example 25: Alternative evanescent color fade sunscreen formulation.
Example 26: Formaldehyde free microencapsulation applied to evanescent formulations
Example 27: Formaldehyde scavenging systems for formaldehyde reduction in evanescent systems

### Additional Embodiments

Example 28: Encapsulated non-evanescent dyes in evanescent color change compositions
Example 29: Encapsulated sunscreen active ingredient in evanescent color change compositions
Example 30: Reduced stainability by encapsulated evanescent color change compositions
Example 31: Encapsulated sunscreen inactive ingredient in evanescent color change compositions
Example 32: Oil-in-water encapsulated evanescent color change compositions
Example 33: Reverse phase water-in-oil encapsulated evanescent color change compositions
Example 34: Environmentally triggered release microencapsulation evanescent color change compositions
Example 35: Entrapped encapsulated evanescent color change compositions
Example 36: Pulverized entrapped encapsulated evanescent color change compositions
Example 37: Formaldehyde-free encapsulated evanescent color change compositions
Example 38: Macro-encapsulated evanescent color change compositions
Example 39: Color-to-color transition evanescent color change compositions
Example 40: Heat activated evanescent color change compositions with thermal free radical initiators
Example 41: Photoinitiated evanescent color change compositions with visible, UV-A, UV-B and UV-C activation
Example 42: Evanescence activated encapsulation release compositions
Example 43: Chemiluminescent activation induced evanescent color change compositions
Example 44: Chemiluminescent evanescent color change compositions
Example 45: Epoxidized soybean oil core material in encapsulate evanescent color change compositions
Example 46: Liquid evanescent color change compositions
Example 47: Powder evanescent color change compositions
Example 48: Neat evanescent color change compositions
Example 49: Purified evanescent color change compositions
Example 50: Scavenger containing evanescent color change compositions
Example 51: Nanometer sized capsule containing evanescent color change compositions
Example 52: Sub-millimeter sized capsule containing evanescent color change compositions
Example 53: Transient color change evanescent color change compositions
Example 54: Stain resistant evanescent color change compositions
Example 55: PEG-modified microencapsulated evanescent color change compositions
Example 56: Multiply encapsulated evanescent color change compositions
Example 57: Surface modified microencapsulated evanescent color change compositions
Example 58: Lotion compatible evanescent color change compositions
Example 59: Irreversible evanescent color change compositions
Example 60: Reversible evanescent color change compositions
Example 61: Catalytic accelerators and evanescent color change compositions
Example 62: Color change rate modulated evanescent color change compositions
Example 63: Rate controlled evanescent color change compositions
Example 64: Timed release evanescent color change compositions
Example 65: Combination therapeutic agent evanescent color change compositions
Example 66: Single core encapsulated evanescent color change compositions
Example 67: Multi-core encapsulated evanescent color change compositions
Example 68: Single photoinitiator evanescent color change compositions
Example 69: Multi-photoinitiator evanescent color change compositions
Example 70: Liquid slurry evanescent color change compositions
Example 71: Powder evanescent color change compositions
Example 72: Fracture resistant microencapsulated evanescent color change compositions
Example 73: Shear susceptible microencapsulated evanescent color change compositions
Example 74: Antioxidant containing microencapsulated evanescent color change compositions
Example 75: Translucent and opaque evanescent color change compositions
Example 76: Heat-stress additives evanescent color change compositions
Example 77: Encapsulation with urea formaldehyde resin
Example 78: Encapsulated and entrapped evanescent color-fade dye systems
Example 79: Evanescent non-FD&C dye systems
Example 80: Evanescent color-fade formulation using one or more photo-initiators
Example 81: Microcapsule Compositions

Embodiments of the present application are directed to microcapsule compositions, manufacturing process, and use of these compositions in skincare applications. The microcapsule comprises of core-shell structure system and dispersible in aqueous system. In some instances, a core contains oil-soluble FDA approved food colorants, a photo-initiator and a matrix that is soluble in oil. There is an emulsifier/surfactant and an outer shell/capsule material that is made of cured polymeric resin. Average particle size of each microcapsule varies, and in some instances ranges from 2-5 microns.

The microencapsulated materials are, in some instances, prepared by using following ingredients: i) an aqueous phase containing from 45-65% by weight of the slurry where an emulsifier is present where from 1% to 10% of the aqueous phase solution; ii) an oil-soluble core made from 20-30% by weight of the slurry, where from 0.1-1% colorants, 1-10% of photo-initiators are present; iii) a shell material constituting 10-20% of the slurry by weight of which 40-60% is pre-polymer resin itself; optionally, iv) an accelerator to control the curing of pre-polymeric shell; and v) a pH controlling agent.

An embodiment of an emulsifier agent is a water-soluble polymer/co-polymer. The emulsifier may include anionic, cationic or nonionic polymers and may be used as an aqueous phase in order to form initial oil in water microemulsion droplets and prevent agglomerations. Some examples of emulsifiers are polystyrenesulfonate, styrene copolymers, polyvinylsulfonates, maleic anhydridestyrene copolymer, maleic anhydride-isobutylene copolymer, maleic anhydride-ethylene copolymer, maleic anhydride-methyl vinyl ether copolymer, polyvinyl alcohol (saponified product), carboxymethyl-modified polyvinyl alcohol, gum arabic, polyacrylates, polyacrylate derivatives, acrylate copolymers, carboxymethyl cellulose, gelatin, pectin, other gelatin derivatives, cellulose sulfate ester salt, and alginic acid. Optionally, the pH of the emulsifier is adjusted with triethanolamine.

FDA approved dyes may be used as a coloring agent for the cosmetics and sunscreen applications for safety regulation. Oil soluble dyes are preferred due to the hydrophobic nature of the matrix in the core and better compatibility. A few examples of FDA approved oil-soluble dyes are D&C Violet 2, D&C Green 6 (anthraquinone-based dyes), D&C Yellow 11 (quinoline based) or D&C Red 17.

A. Micro-Encapsulation with Urea Formaldehyde (UF) Resin
1: The oil soluble core materials, (containing D&C Violet 2, TPO-L, Octyl dodecanol) are mixed and heated at around 110-125° C. The aqueous phase containing the emulsifiers (maleic anhydride-methyl vinyl ether copolymer, MVEMAC) are heated separately at approximately 60-70° C. The internal core phase is poured in a timely manner into the aqueous phase under high shearing condition (8000-10,000 rpm) controlled by a homogenizer to generate the micron sized o/w based emulsion. The rate of addition of core oil phase depends on the volume of the reaction. Thereafter, shell forming PMU pre-polymeric resin solution is added slowly to ensure the desired rate of wall formation. Urea-formaldehyde pre-polymers are soluble in water, available commercially with varying density. After the complete addition of resin, the homogenizer is replaced with an overhead mechanical mixer with three-blade propeller and the polymerization/curing is continued until the completion of wall formation. Curing reaction is done around 65-75° C. After, end of the reaction, the product is cooled, filtered and tested. Reaction rate can be controlled by pH modification or by using an accelerator/initiator. At the end of the process, in the microencapsulated slurry, solid content is between 40-50%.
   Composition of the present microencapsulated slurry is provided as an example:

| | % |
|---|---|
| Emulsifier, 10% | 54.56 |
| 2-Octyl 1-Dodecanol | 20.99 |
| D&C Violet #2 | 0.31 |
| Photoinitiator | 3.15 |
| PMU/UF resin | 10.49 |
| Deionized Water | 10.49 |
| Total | 100.00 |

2: In another embodiment, the oil soluble core materials, (containing D&C green 6, TPO-L, Octyl dodecanol) are mixed and heated at around 110-125 C. The water-soluble PMU pre polymer resin is added to the aqueous phase containing the emulsifiers (maleic anhydride-methyl vinyl ether copolymer, MVEMAC) and are heated at approximately 60-70° C. The internal core phase is poured in a timely manner into the aqueous phase under high shearing condition (8000-10,000 rpm) controlled by a homogenizer to generate the micron sized o/w based emulsion. The rate of addition of core oil phase depends on the volume of the reaction. After complete addition of the core-phase, the homogenizer is replaced with an overhead mixer with three-blade propeller and the polymerization/curing is continued until the completion of wall formation. Curing reaction is done around 65-75° C. Reaction rate can be controlled by pH modification or by using an accelerator/initiator. At the end of the process, in the microencapsulated slurry, solid content is between 40-50%.
3: In another embodiment, the oil soluble core materials, (containing D&C violet 2, TPO-L, Octyl dodecanol) are mixed and heated at around 110-125° C. pH of the aqueous phase is adjusted to 3.3-3.4 with triethanolamine. The aqueous phase containing the emulsifiers (maleic anhydride-methyl vinyl ether copolymer, MVEMAC) and are heated at approximately 60-70° C. The internal core phase is poured in a timely manner into the aqueous phase under high shearing condition (8000-10,000 rpm) controlled by a homogenizer to generate the micron sized o/w based emulsion. The rate of addition of core oil phase depends on the volume of the reaction. Thereafter, shell forming PMU prepolymeric resin solution is added slowly to ensure the desired rate of wall formation. After the complete addition of resin, the homogenizer is replaced with an overhead mixer and the polymerization/curing is continued until the completion of wall formation. After an hour, p-toulene sulphonic acid is added as an acid catalyst to adjust the pH around 3.0. Curing reaction is done around 65-75° C. for 3-5 h. At the end of the process, in the microencapsulated slurry, solid content is between 40-50%.
4: Above three experimental procedures for making micro emulsion (Example 1-3), are tested with varying temperature during curing reaction. The temperature range varies from 45-85° C.
5: Reaction condition of the microemulsion was tested by varying temperature during curing reaction of the urea-formaldehyde. The temperature range varies from 45-85° C. Depending on the type of prepolymer, the procedure worked best with 15 minutes homogenization at 60° C., followed by 3 h of microencapsulation reaction.
6: Curing condition is also varied against different pH (2.8-4.0) and using different type of acid catalysts. The acid catalyst can be selected from Dodecyl sulphonic acid, P-toluene sulfonic acid, citric acid, phosphoric acid,
7: Above three microencapsulation procedures are tested in presence of using ammonium salt accelerator for UF resin, for example 10% ammonium chloride, ammonium sulphate to facilitate the polymerization reaction of UF-resin during capsule formation.
8: Application in sunscreen formulation:

Testing: Micro-encapsulated slurry containing the color-block element is tested for stability, functionality in presence of sunlight as well as under handheld UV light (UVA and UV-B). Similar test is run with commercial standard sunscreen lotion by adding 3-10% loading of the microencapsulated slurry and tested under sunlight and handheld UV-light. For example, Coppertone Ultraguard™ sunscreen lotion with SPF 50 loaded with 10% microencapsulated slurry was violet in color at the room temperature before any exposure to sunlight. In presence of UV-A & UV-B with UVI 5, violet color faded and finally turned into colorless/slight yellow with 5-10 minutes. Effectiveness of the color fading varied with different sunscreen formulation with respect to their SPF values (15-100), precisely within SPF 30-70. Inherent sunscreen formulation (both active and inactive ingredients) also varies depending on the final application and target users age range. If color block microencapsulated slurry is used approximately 3-10%, sucy 5-6%, in the final sunscreen formulation, final sunscreen formulation is:

| Final sunscreen lotion | Range, % | % |
|---|---|---|
| Color block microencapsulated | 3-10 | 5 |
| slurry | | |
| Active ingredients | 25-40 | 32 |
| Inactive ingredients | 50-75 | 63 |
| Total | | 100.00 |

9: Time dependent Shelf-life stability study:

Shelf stability at room temperature of the color-blocked sunscreen formulation by added microencapsulated slurry, is also tested for short term and long term time cycle in order to establish the effectiveness, and reliability of the final product. Depending on the formulation and nature of the slurry and sunscreen formulation used, the final color-blocked sunscreen lotion shows shelf stability up to two years.

Notwithstanding the appended clauses, the disclosure is also defined by the following clauses:
1. An evanescent color change composition comprising:

a colored pigment; and

a color changer that changes the colored pigment from colored to colorless in response to an applied stimulus to the color changer.
2. The color change composition according to clause 1, wherein the colored pigment is a dye.
3. The color change composition according to clause 2, wherein the colored pigment is a hydrophobic dye.
4. The color change composition according to clause 2, wherein the colored pigment is an anthraquinone dye.
5. The color change composition according to clause 2, wherein the colored pigment is an oil soluble dye.
6. The color change composition according to clause 2, wherein the colored pigment is a carotenoid.
7. The color change composition according to any of clauses 1 to 6, wherein the colored pigment is chemically coupled to a long chain hydrocarbon.
8. The color change composition according to any of clauses 1 to 6, wherein the colored pigment is chemically coupled to a polyalkylene glycol.
9. The color change composition according to any of clauses 1 to 8, wherein the color changer changes the colored pigment from colored to colorless in response to light.
10. The color change composition according to clause 9, wherein the light is ultraviolet (UV) light or visible light.
11. The color change composition according to clause 9, wherein the light has a wavelength from 200 nm to 400 nm.
12. The color change composition according to clause 9, wherein the light has a wavelength from 400 nm to 700 nm.
13. The color change composition according to clause 9, wherein the light has a wavelength from 700 nm to 1200 nm.
14. The color change composition according to any of clauses 1 to 13, wherein the color changer is a free radical photoinitiator.
15. The color change composition according to clause 14, wherein the free radical photoinitiator is a phosphine oxide, an α-amino ketone photoinitiator, a titanocene and an azide compound.
16. The color change composition according to clause 15, wherein the free radical photoinitiator comprises a phosphine oxide selected from the group consisting of bis(2,6-dimethoxybenzoyl)-(2-methylpropyl)phosphine oxide, bis(2,4,6-trimethylbenzoyl)-(2-methylpropyl)phosphine oxide, bis(2,6-dimethoxybenzoyl)-(2,4,4-trimethylpentyl)phosphine oxide, bis(2,4,6-trimethylbenzoyl)-(2,4,4-trimethylpentyl)phosphine oxide, (2,6-dimethoxybenzoyl)-(2,4,6-trimethylbenzoyl)-(2-methylpropyl)phosphine oxide, phenylbis(3-{[2-(allyloxy)ethoxy]methyl}-2,4,6-trimethylbenzoyl)phosphine oxide, phenylbis{4-[2-(2-Methoxy-ethoxy)-ethoxy]-2,6-dimethyl-benzoyl}phosphine oxide, phenylbis{3-[2-(2-Methoxy-ethoxy)-ethoxymethyl]-2,4,6-trimethyl-benzoyl}phosphine oxide, phenylbis(2,4,6-trimethyl-benzoyl)-4-[2-(2-methoxyethoxy)-ethoxy]phosphine oxide, phenylbis(2,4,6-trimethyl-benzoyl)-4-methoxy-phosphine oxide, phenylbis(2,4,6-trimethylbenzoyl)-phosphine oxide, 2,4,6-trimethylbenzoyldiphenylphosphine oxide, 2,4,6-trimethylbenzoylphenyl phosphinate, bis(2-methylpropyl)(2,6-dimethoxybenzoyl)phosphine oxide, bis(2-methylpropyl)(2,4,6-trimethylbenzoyl)phosphine oxide, 2,4,6-trimethylbenzoylphenylphosphinic acid ethyl ester, bis(2,4,4-trimethylpentyl)(2,6-dimethoxybenzoyl)phosphine oxide, bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide, bis-(2-chloro-1-naphthoyl)-2,5-dimethylphenylphosphine oxide and combinations thereof.
17. The color change composition according to any of clauses 1 to 13, wherein the color changer is a singlet oxygen photosensitizer.
18. The color change composition according to clause 17, wherein the singlet oxygen photosensitizer absorbs light of 400 nm or more.
19. The color change composition according to clause 17 or 18, wherein the singlet oxygen photosensitizer is a polycyclic aromatic hydrocarbon, cyanine, fluroscein, anthracene or porphyrin.
20. The color change composition according to any of clauses 17 to 19, wherein the singlet oxygen photosensitizer is selected from the group consisting of acridine, acetonaphthone, anthra[1,9-bc:4,10-b′c′]dichromene, 9,10-anthracenedipropionate ion, aluminum(III) sulfophthalocyanine, anthracene, angelicin, anthracenesulfonate ion, acetophenone, 9,10-athraquinone, allylthiourea, bacteriochlorophyll a, benzo[1,2,3-kl:4,5,6k′l′]dixanthene, biphenyl, benzophenone, bilirubin, bilirubin dianion, benoxaprofen, β-carotene, cadmium(II) 1-(2-hydroxyphenylazo)-2-naphtholate, chlorophyll a, camphoroquinone, chlorpromazine, 9,10-dicyanoanthracene, 9,10-dimethylanthracene, 4,7-dimethylallopsoralen, 9,10-dimethylbenz[a]anthracene, 1,4-dimethoxy-9,10-diphenylanthracene, 2,5-dimethylfuran, 4,4′-dimethoxythiobenzophenone, 1,8-dinaphthalene thiophene, diacenaphtho[1,2-b:1′,2′-d]thiophene, 3-(3,4-dihydroxyphenyl)alanine, 9,10-diphenylanthracene, 1,4-diphenyl-1,3-butadiene, 1,3-diphenylisobenzofuran, 2,5-diphenylfuran, 1,6-diphenyl-1,3,5-hexatriene, 1,8-diphenyl-1,3,5,7-octatetraene, 2,5-di-tert-butylfuran, eosin (tetrabromofluorescein), erythrosin (tetraiodofluorescein), ergosterol, furfuryl alcohol, fluorescein, heterocoerdianthrone, histidine, hematoporphyrin, hypericin, imidazole, 4′-methoxyacetophenone, methylene blue, mesodiphenylbenzhelianthrene, mesodiphenylhelianthrene, 1-methylnaphthalene, methoxypsoralen, 2-methyl-2-pentene, mesoporphyrin diethyl ester, mesoporphyrin dimethyl ester, 10-Methyl-9-acridinethione, naphthalene, palladium(II) tetraphenylporphyrin, palladium(II) tetrakis(4-sulfonatophenyl)porphyrin, perylene, pheophytin a, protoporphyrin, protoporphyrin dimethyl ester, 2,7,12,17-tetrapropylporphycene, platinum(II) diazido(2,2′-bipyridine), platinum(II) (1,10-phenanthroline)(tert-butylcatechol), platinum(II) (1,10-phenanthroline)(2,3-naphthalenediol), pyrene, phenazine, Rose Bengal (tetrachlorotetraiodofluorescein), Rose Bengal ethyl ester, retinal, riboflavin, N,N-dimethyl-4-nitrosoaniline, rubrene (5,6,11,12-tetraphenylnaphthacene), 2,2,6,6-tetramethylpiperidin-4-ol, tetracene, tetra(3-hydroxyphenyl)porphyrin, tetra(4-hydroxyphenyl)porphyrin, 2,3-Dimethyl-2-butene (tetramethylethylene), tetra(4-N-methylpyridyl)porphyrin, tetraphenylbacteriochlorin, tetraphenylcyclopentadienone, tetraphenylporphyrin, tetra(4-sulfonatophenyl)porphyrin, tryptophan, uroporphyrin I, zinc(II) tetraphenylporphyrin, zinc(II) 2-(4,5-diphenylimidazol-2-yl)azo-5-methylbenzoate and zinc(II) 2-(4,5-diphenylimidazol-2-yl)azo-4-nitrophenolate and combinations thereof.
21. The color change composition according to any of clauses 1 to 20, wherein the color changer initiates the decomposition of the colored pigment.
22. The color change composition according to clause 21, wherein the colored pigment decomposes in 10 minutes or less after application of the applied stimulus.
23. The color change composition according to clause 21, wherein the colored pigment decomposes in 5 minutes or less after application of the applied stimulus.
24. The color change composition according to any of clauses 1 to 23, wherein the colored pigment and color changer are microencapsulated.
25. The color change composition according to clause 24, wherein the colored pigment and color changer are microencapsulated with a maleic anhydride copolymer or a urea formaldehyde resin.
26. The color change composition according to any of clauses 1 to 25, further comprising a solvent.
27. The color change composition according to clause 26, wherein the solvent is a long chain hydrocarbon alcohol.
28. The color change composition according to clause 27, wherein the long chain hydrocarbon alcohol is selected from the group consisting of 1-tetradecanol, 1-hexadecanol, 1-octadecanol, 1-eicosanol, 1-docosanol, oils, mineral oil, petroleum jelly, corn oil, canola oil, castor oil, and mixtures thereof.
29. The color change composition according to any of clauses 1 to 28, further comprising a UV absorber.
30. The color change composition according to clause 29, wherein the UV absorber is a UV-A absorber.
31. The color change composition according to clause 29, wherein the UV absorber is a UV-B absorber.
32. The color change composition according to clause 29, wherein the UV absorber is a UV-C absorber.
33. The color change composition according to any of clauses 1 to 32, wherein the molar ratio of color changer to colored pigment is from 0.1 to 50.
34. The color change composition according to clause 33, wherein the molar ratio of color changer to colored pigment is 10.
35. A liquid evanescent color change composition comprising:

a solvent; and

a color change composition comprising:
a colored pigment; and
a color changer that changes the colored pigment from colored to colorless in response to light.
36. The composition according to clause 35, wherein the composition is formulated for coating a surface.
37. The composition according to clause 36, wherein the composition is a sunscreen.
38. The composition according to clause 37, wherein the sunscreen comprises a UV absorber selected from the group consisting of a UV-A absorber, a UV-B absorber and a UV-C absorber.
39. The composition according to clause 38, wherein the UV absorber comprises titanium dioxide, zinc oxide, p-Aminobenzoic acid (PABA), octyldimethyl-PABA, phenylbenzimidazole sulfonic acid, 2-ethoxyethyl p-methoxycinnamate, dioxybenzone, oxybenzone, homomethyl salicylate, menthyl anthranilate, 2-cyano-3,3-diphenyl acrylic acid 2-ethylhexylester, 2-ethylhexyl-paramethoxycinnamate, 2-ethylhexyl salicylate, 3-benzoyl-4-hydroxy-6-methoxybenzenesulfonic acid, triethanolamine salicylate, 1-(4-methoxyphenyl)-3-(4-tert-butyl phenyl)propane-1,3-dione, terephthalylidene dicamphor sulfonic acid, 4-methylbenzylidene camphor, methylene bis-benzotriazolyl tetramethylbutylphenol, tris-biphenyl triazine, disodium phenyl dibenzimidazole tetrasulfonate, drometrizole trisiloxane, sodium dihydroxy dimethoxy disulfobenzophenone, ethylhexyl triazone, diethylamino hydroxybenzoyl hexyl benzoate, diethylhexyl butamido triazone, dimethico-diethylbenzalmalonate, isopentyl-4-methoxycinnamate or a combination or mixture thereof.
40. The composition according to any of clauses 35 to 39, wherein the colored pigment is a dye.
41. The composition according to clause 40, wherein the colored pigment is a hydrophobic dye.
42. The composition according to clause 40, wherein the colored pigment is an anthraquinone dye.
43. The composition according to clause 40, wherein the colored pigment is an oil soluble dye.
44. The composition according to clause 40, wherein the colored pigment is a carotenoid.
45. The composition according to any of clauses 35 to 44, wherein the colored pigment is chemically coupled to a long chain hydrocarbon.
46. The composition according to any of clauses 35 to 44, wherein the colored pigment is chemically coupled to a polyalkylene glycol.
47. The composition according to clause 46, wherein the light is ultraviolet (UV) light or visible light.
48. The composition according to clause 46, wherein the light has a wavelength from 200 nm to 400 nm.
49. The composition according to clause 46, wherein the light has a wavelength from 400 nm to 700 nm.
50. The composition according to clause 46, wherein the light has a wavelength from 700 nm to 1200 nm.
51. The composition according to any of clauses 35 to 50, wherein the color changer is a free radical photoinitiator.
52. The composition according to clause 51, wherein the free radical photoinitiator is a phosphine oxide, an α-amino ketone photoinitiator, a titanocene and an azide compound.
53. The composition according to clause 52, wherein the free radical photoinitiator comprises a phosphine oxide selected from the group consisting of bis(2,6-dimethoxybenzoyl)-(2-methylpropyl)phosphine oxide, bis(2,4,6-trimethylbenzoyl)-(2-methylpropyl)phosphine oxide, bis(2,6-dimethoxybenzoyl)-(2,4,4-trimethylpentyl)phosphine oxide, bis(2,4,6-trimethylbenzoyl)-(2,4,4-trimethylpentyl)phosphine oxide, (2,6-dimethoxybenzoyl)-(2,4,6-trimethylbenzoyl)-(2-methylpropyl)phosphine oxide, phenylbis(3-{[2-(allyloxy)ethoxy]methyl}-2,4,6-trimethylbenzoyl)phosphine oxide, phenylbis{4-[2-(2-Methoxy-ethoxy)-ethoxy]-2,6-dimethyl-benzoyl}phosphine oxide, phenylbis{3-[2-(2-Methoxy-ethoxy)-ethoxymethyl]-2,4,6-trimethyl-benzoyl}phosphine oxide, phenylbis(2,4,6-trimethyl-benzoyl)-4-[2-(2-methoxyethoxy)-ethoxy]phosphine oxide, phenylbis(2,4,6-trimethyl-benzoyl)-4-methoxy-phosphine oxide, phenylbis(2,4,6-trimethylbenzoyl)-phosphine oxide, 2,4,6-trimethylbenzoyldiphenylphosphine oxide, 2,4,6-trimethylbenzoylphenyl phosphinate, bis(2-methylpropyl)(2,6-dimethoxybenzoyl)phosphine oxide, bis(2-methylpropyl)(2,4,6-trimethylbenzoyl)phosphine oxide, 2,4,6-trimethylbenzoylphenylphosphinic acid ethyl ester, bis(2,4,4-trimethylpentyl)(2,6-dimethoxybenzoyl)phosphine oxide, bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide, bis-(2-chloro-1-naphthoyl)-2,5-dimethylphenylphosphine oxide and combinations thereof.
54. The sunscreen composition according to any of clauses 35 to 50, wherein the color changer is a singlet oxygen photosensitizer.
55. The composition according to clause 54 wherein the singlet oxygen photosensitizer absorbs light of 400 nm or more.
56. The composition according to clause 54 or 55, wherein the singlet oxygen photosensitizer is a polycyclic aromatic hydrocarbon, cyanine, fluroscein, anthracene or porphyrin.
57. The composition according to any of clauses 54 to 56, wherein the singlet oxygen photosensitizer is selected from the group consisting of acridine, acetonaphthone, anthra[1,9-bc:4,10-b′c′]dichromene, 9,10-anthracenedipropionate ion, aluminum(III) sulfophthalocyanine, anthracene, angelicin, anthracenesulfonate ion, acetophenone, 9,10-athraquinone, allylthiourea, bacteriochlorophyll a, benzo[1,2,3-kl:4,5,6k′l′]dixanthene, biphenyl, benzophenone, bilirubin, bilirubin dianion, benoxaprofen, β-carotene, cadmium(II) 1-(2-hydroxyphenylazo)-2-naphtholate, chlorophyll a, camphoroquinone, chlorpromazine, 9,10-dicyanoanthracene, 9,10-dimethylanthracene, 4,7-dimethylallopsoralen, 9,10-dimethylbenz[a]anthracene, 1,4-dimethoxy-9,10-diphenylanthracene, 2,5-dimethylfuran, 4,4′-dimethoxythiobenzophenone, 1,8-dinaphthalene thiophene, diacenaphtho[1,2-b:1′,2′-d]thiophene, 3-(3,4-dihydroxyphenyl)alanine, 9,10-diphenylanthracene, 1,4-diphenyl-1,3-butadiene, 1,3-diphenylisobenzofuran, 2,5-diphenylfuran, 1,6-diphenyl-1,3,5-hexatriene, 1,8-diphenyl-1,3,5,7-octatetraene, 2,5-di-tert-butylfuran, eosin (tetrabromofluorescein), erythrosin (tetraiodofluorescein), ergosterol, furfuryl alcohol, fluorescein, heterocoerdianthrone, histidine, hematoporphyrin, hypericin, imidazole, 4′-methoxyacetophenone, methylene blue, mesodiphenylbenzhelianthrene, mesodiphenylhelianthrene, 1-methylnaphthalene, methoxypsoralen, 2-methyl-2-pentene, mesoporphyrin diethyl ester, mesoporphyrin dimethyl ester, 10-Methyl-9-acridinethione, naphthalene, palladium(II) tetraphenylporphyrin, palladium(II) tetrakis(4-sulfonatophenyl)porphyrin, perylene, pheophytin a, protoporphyrin, protoporphyrin dimethyl ester, 2,7,12,17-tetrapropylporphycene, platinum(II) diazido(2,2′-bipyridine), platinum(II) (1,10-phenanthroline)(tert-butylcatechol), platinum(II) (1,10-phenanthroline)(2,3-naphthalenediol), pyrene, phenazine, Rose Bengal (tetrachlorotetraiodofluorescein), Rose Bengal ethyl ester, retinal, riboflavin, N,N-dimethyl-4-nitrosoaniline, rubrene (5,6,11,12-tetraphenylnaphthacene), 2,2,6,6-tetramethylpiperidin-4-ol, tetracene, tetra(3-hydroxyphenyl)porphyrin, tetra(4-hydroxyphenyl)porphyrin, 2,3-Dimethyl-2-butene (tetramethylethylene), tetra(4-N-methylpyridyl)porphyrin, tetraphenylbacteriochlorin, tetraphenylcyclopentadienone, tetraphenylporphyrin, tetra(4-sulfonatophenyl)porphyrin, tryptophan, uroporphyrin I, zinc(II) tetraphenylporphyrin, zinc(II) 2-(4,5-diphenylimidazol-2-yl)azo-5-methylbenzoate and zinc(II) 2-(4,5-diphenylimidazol-2-yl)azo-4-nitrophenolate and combinations thereof.
58. The composition according to any of clauses 35 to 57, wherein the color changer initiates the decomposition of the colored pigment.
59. The composition according to clause 58, wherein the colored pigment decomposes in 10 minutes or less after application of the applied stimulus.
60. The composition according to clause 59, wherein the colored pigment decomposes in 5 minutes or less after application of the applied stimulus.
61. The composition according to any of clauses 35 to 60, wherein the colored pigment and color changer are microencapsulated.
62. The composition according to clause 61, wherein the colored pigment and color changer are microencapsulated with a maleic anhydride copolymer or a urea formaldehyde resin.
63. The composition according to any of clauses 35 to 62, wherein the solvent comprises a long chain hydrocarbon alcohol.
64. The composition according to clause 63, wherein the long chain hydrocarbon alcohol is selected from the group consisting of 1-tetradecanol, 1-hexadecanol, 1-octadecanol, 1-eicosanol, 1-docosanol, oils, mineral oil, petroleum jelly, corn oil, canola oil, castor oil, and mixtures thereof.
65. The composition according to any of clauses 35 to 64, wherein the molar ratio of color changer to colored pigment is from 0.1 to 50.
66. The composition according to clause 65, wherein the molar ratio of color changer to colored pigment is 10.
67. A method of making an evanescent color change composition, the method comprising:

combining a colored pigment with a color change that changes the colored pigment from colored to colorless upon application of an applied stimulus to the color changer.
68. The method according to clause 67, wherein the colored pigment is a dye.
69. The method according to clause 68, wherein the colored pigment is a hydrophobic dye.
70. The method according to clause 68, wherein the colored pigment is an anthraquinone dye.
71. The method according to clause 68, wherein the colored pigment is an oil soluble dye.
72. The method according to clause 68, wherein the colored pigment is a carotenoid.
73. The method according to any of clauses 67 to 72, wherein the colored pigment is chemically coupled to a long chain hydrocarbon.
74. The method according to any of clauses 67 to 72, wherein the colored pigment is chemically coupled to a polyalkylene glycol.
75. The method according to any of clauses 67 to 72, wherein the color changer changes the colored pigment from colored to colorless in response to light.
76. The method according to clause 75, wherein the light is ultraviolet (UV) light or visible light.
77. The method according to clause 75, wherein the light has a wavelength from 200 nm to 400 nm.
78. The method according to clause 75, wherein the light has a wavelength from 400 nm to 700 nm.
79. The method according to clause 75, wherein the light has a wavelength from 700 nm to 1200 nm.
80. The method according to any of clauses 67 to 79, wherein the color changer is a free radical photoinitiator.
81. The method according to clause 80, wherein the free radical photoinitiator is a phosphine oxide, an α-amino ketone photoinitiator, a titanocene and an azide compound.
82. The method according to clause 81, wherein the free radical photoinitiator comprises a phosphine oxide selected from the group consisting of bis(2,6-dimethoxybenzoyl)-(2-methylpropyl)phosphine oxide, bis(2,4,6-trimethylbenzoyl)-(2-methylpropyl)phosphine oxide, bis(2,6-dimethoxybenzoyl)-(2,4,4-trimethylpentyl)phosphine oxide, bis(2,4,6-trimethylbenzoyl)-(2,4,4-trimethylpentyl)phosphine oxide, (2,6-dimethoxybenzoyl)-(2,4,6-trimethylbenzoyl)-(2-methylpropyl)phosphine oxide, phenylbis(3-{[2-(allyloxy)ethoxy]methyl}-2,4,6-trimethylbenzoyl)phosphine oxide, phenylbis{4-[2-(2-Methoxy-ethoxy)-ethoxy]-2,6-dimethyl-benzoyl}phosphine oxide, phenylbis{3-[2-(2-Methoxy-ethoxy)-ethoxymethyl]-2,4,6-trimethyl-benzoyl}phosphine oxide, phenylbis(2,4,6-trimethyl-benzoyl)-4-[2-(2-methoxyethoxy)-ethoxy]phosphine oxide, phenylbis(2,4,6-trimethyl-benzoyl)-4-methoxy-phosphine oxide, phenylbis(2,4,6-trimethylbenzoyl)-phosphine oxide, 2,4,6-trimethylbenzoyldiphenylphosphine oxide, 2,4,6-trimethylbenzoylphenyl phosphinate, bis(2-methylpropyl)(2,6-dimethoxybenzoyl)phosphine oxide, bis(2-methylpropyl)(2,4,6-trimethylbenzoyl)phosphine oxide, 2,4,6-trimethylbenzoylphenylphosphinic acid ethyl ester, bis(2,4,4-trimethylpentyl)(2,6-dimethoxybenzoyl)phosphine oxide, bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide, bis-(2-chloro-1-naphthoyl)-2,5-dimethylphenylphosphine oxide and combinations thereof.
83. The method according to any of clauses 67 to 79, wherein the color changer is a singlet oxygen photosensitizer.
84. The method according to clause 83, wherein the singlet oxygen photosensitizer absorbs light of 400 nm or more.
85. The method according to clause 83 or 84, wherein the singlet oxygen photosensitizer is a polycyclic aromatic hydrocarbon, cyanine, fluroscein, anthracene or porphyrin.
86. The method according to any of clauses 82 to 85, wherein the singlet oxygen photosensitizer is selected from the group consisting of acridine, acetonaphthone, anthra[1,9-bc:4,10-b′c′]dichromene, 9,10-anthracenedipropionate ion, aluminum(III) sulfophthalocyanine, anthracene, angelicin, anthracenesulfonate ion, acetophenone, 9,10-athraquinone, allylthiourea, bacteriochlorophyll a, benzo[1,2,3-kl:4,5,6k′l′]dixanthene, biphenyl, benzophenone, bilirubin, bilirubin dianion, benoxaprofen, β-carotene, cadmium(II) 1-(2-hydroxyphenylazo)-2-naphtholate, chlorophyll a, camphoroquinone, chlorpromazine, 9,10-dicyanoanthracene, 9,10-dimethylanthracene, 4,7-dimethylallopsoralen, 9,10-dimethylbenz[a]anthracene, 1,4-dimethoxy-9,10-diphenylanthracene, 2,5-dimethylfuran, 4,4′-dimethoxythiobenzophenone, 1,8-dinaphthalene thiophene, diacenaphtho[1,2-b:1′,2′-d]thiophene, 3-(3,4-dihydroxyphenyl)alanine, 9,10-diphenylanthracene, 1,4-diphenyl-1,3-butadiene, 1,3-diphenylisobenzofuran, 2,5-diphenylfuran, 1,6-diphenyl-1,3,5-hexatriene, 1,8-diphenyl-1,3,5,7-octatetraene, 2,5-di-tert-butylfuran, eosin (tetrabromofluorescein), erythrosin (tetraiodofluorescein), ergosterol, furfuryl alcohol, fluorescein, heterocoerdianthrone, histidine, hematoporphyrin, hypericin, imidazole, 4′-methoxyacetophenone, methylene blue, mesodiphenylbenzhelianthrene, mesodiphenylhelianthrene, 1-methylnaphthalene, methoxypsoralen, 2-methyl-2-pentene, mesoporphyrin diethyl ester, mesoporphyrin dimethyl ester, 10-Methyl-9-acridinethione, naphthalene, palladium(II) tetraphenylporphyrin, palladium(II) tetrakis(4-sulfonatophenyl)porphyrin, perylene, pheophytin a, protoporphyrin, protoporphyrin dimethyl ester, 2,7,12,17-tetrapropylporphycene, platinum(II) diazido(2,2′-bipyridine), platinum(II) (1,10-phenanthroline)(tert-butylcatechol), platinum(II) (1,10-phenanthroline)(2,3-naphthalenediol), pyrene, phenazine, Rose Bengal (tetrachlorotetraiodofluorescein), Rose Bengal ethyl ester, retinal, riboflavin, N,N-dimethyl-4-nitrosoaniline, rubrene (5,6,11,12-tetraphenylnaphthacene), 2,2,6,6-tetramethylpiperidin-4-ol, tetracene, tetra(3-hydroxyphenyl)porphyrin, tetra(4-hydroxyphenyl)porphyrin, 2,3-Dimethyl-2-butene (tetramethylethylene), tetra(4-N-methylpyridyl)porphyrin, tetraphenylbacteriochlorin, tetraphenylcyclopentadienone, tetraphenylporphyrin, tetra(4-sulfonatophenyl)porphyrin, tryptophan, uroporphyrin I, zinc(II) tetraphenylporphyrin, zinc(II) 2-(4,5-diphenylimidazol-2-yl)azo-5-methylbenzoate and zinc(II) 2-(4,5-diphenylimidazol-2-yl)azo-4-nitrophenolate and combinations thereof.
87. The method according to any of clauses 67 to 86, wherein the color changer initiates the decomposition of the colored pigment.
88. The method according to clause 87 wherein the colored pigment decomposes in 10 minutes or less after application of the applied stimulus.
89. The method according to clause 87, wherein the colored pigment decomposes in 5 minutes or less after application of the applied stimulus.
90. The method according to any of clauses 67 to 89, wherein the colored pigment and color changer are microencapsulated.
91. The method according to clause 90, wherein the colored pigment and color changer are microencapsulated with a maleic anhydride copolymer or a urea formaldehyde resin.
92. The method according to any of clauses 67 to 91, further comprising a combining the components with a solvent.
93. The method according to clause 92, wherein the solvent comprises a long chain hydrocarbon alcohol.
94. The method according to clause 93, wherein the long chain hydrocarbon alcohol is selected from the group consisting of 1-tetradecanol, 1-hexadecanol, 1-octadecanol, 1-eicosanol, 1-docosanol, oils, mineral oil, petroleum jelly, corn oil, canola oil, castor oil, and mixtures thereof.
95. The method according to any of clauses 67 to 94, further comprising combining the components with a UV absorber.
96. The method according to clause 95, wherein the UV absorber is a UV-A absorber.
97. The method according to clause 95, wherein the UV absorber is a UV-B absorber.
98. The method according to clause 95, wherein the UV absorber is a UV-C absorber.
99. The method according to any of clauses 67 to 98, wherein the molar ratio of color changer to colored pigment is from 0.1 to 50.
100. The method according to clause 99, wherein the molar ratio of color changer to colored pigment is 10.
101. A method of making an evanescent color change sunscreen composition, the method comprising combining a dispersion comprising a UV absorber with an evanescent color change composition to form the color change sunscreen composition, wherein the color change composition comprises:

a colored pigment; and

a color changer that changes the colored pigment from colored to colorless in response to light.
102. The method according to clause 101, wherein the UV absorber is a UV-A absorber.
103. The method according to clause 101, wherein the UV absorber is a UV-B absorber.
104. The method according to clause 101, wherein the UV absorber is a UV-C absorber.
105. The method according to clause 101, wherein the UV absorber comprises titanium dioxide, zinc oxide, p-Aminobenzoic acid (PABA), octyldimethyl-PABA, phenylbenzimidazole sulfonic acid, 2-ethoxyethyl p-methoxycinnamate, dioxybenzone, oxybenzone, homomethyl salicylate, menthyl anthranilate, 2-cyano-3,3-diphenyl acrylic acid 2-ethylhexylester, 2-ethylhexyl-paramethoxycinnamate, 2-ethylhexyl salicylate, 3-benzoyl-4-hydroxy-6-methoxybenzenesulfonic acid, triethanolamine salicylate, 1-(4-methoxyphenyl)-3-(4-tert-butyl phenyl)propane-1,3-dione, terephthalylidene dicamphor sulfonic acid, 4-methylbenzylidene camphor, methylene bis-benzotriazolyl tetramethylbutylphenol, tris-biphenyl triazine, disodium phenyl dibenzimidazole tetrasulfonate, drometrizole trisiloxane, sodium dihydroxy dimethoxy disulfobenzophenone, ethylhexyl triazone, diethylamino hydroxybenzoyl hexyl benzoate, diethylhexyl butamido triazone, dimethico-diethylbenzalmalonate, isopentyl-4-methoxycinnamate or a combination or mixture thereof.
106. The method according to any of clauses 101 to 105, wherein the colored pigment is a dye.
107. The method according to clause 106, wherein the colored pigment is a hydrophobic dye.
108. The method according to clause 106, wherein the colored pigment is an anthraquinone dye.
109. The method according to clause 106, wherein the colored pigment is an oil soluble dye.
110. The method according to clause 106, wherein the colored pigment is a carotenoid.
111. The method according to any of clauses 101 to 110, wherein the colored pigment is chemically coupled to a long chain hydrocarbon.
112. The method according to any of clauses 101 to 110, wherein the colored pigment is chemically coupled to a polyalkylene glycol.
113. The method according to clause 112, wherein the light is ultraviolet (UV) light or visible light.
114. The method according to clause 101, wherein the light has a wavelength from 200 nm to 400 nm.
115. The method according to clause 101, wherein the light has a wavelength from 400 nm to 700 nm.
116. The method according to clause 101, wherein the light has a wavelength from 700 nm to 1200 nm.
117. The method according to any of clauses 101 to 116, wherein the color changer is a free radical photoinitiator.
118. The method according to clause 117, wherein the free radical photoinitiator is a phosphine oxide, an α-amino ketone photoinitiator, a titanocene and an azide compound.
119. The method according to clause 118, wherein the free radical photoinitiator comprises a phosphine oxide selected from the group consisting of bis(2,6-dimethoxybenzoyl)-(2-methylpropyl)phosphine oxide, bis(2,4,6-trimethylbenzoyl)-(2-methylpropyl)phosphine oxide, bis(2,6-dimethoxybenzoyl)-(2,4,4-trimethylpentyl)phosphine oxide, bis(2,4,6-trimethylbenzoyl)-(2,4,4-trimethylpentyl)phosphine oxide, (2,6-dimethoxybenzoyl)-(2,4,6-trimethylbenzoyl)-(2-methylpropyl)phosphine oxide, phenylbis(3-{[2-(allyloxy)ethoxy]methyl}-2,4,6-trimethylbenzoyl)phosphine oxide, phenylbis{4-[2-(2-Methoxy-ethoxy)-ethoxy]-2,6-dimethyl-benzoyl}phosphine oxide, phenylbis{3-[2-(2-Methoxy-ethoxy)-ethoxymethyl]-2,4,6-trimethyl-benzoyl}phosphine oxide, phenylbis(2,4,6-trimethyl-benzoyl)-4-[2-(2-methoxyethoxy)-ethoxy]phosphine oxide, phenylbis(2,4,6-trimethyl-benzoyl)-4-methoxy-phosphine oxide, phenylbis(2,4,6-trimethylbenzoyl)-phosphine oxide, 2,4,6-trimethylbenzoyldiphenylphosphine oxide, 2,4,6-trimethylbenzoylphenyl phosphinate, bis(2-methylpropyl)(2,6-dimethoxybenzoyl)phosphine oxide, bis(2-methylpropyl)(2,4,6-trimethylbenzoyl)phosphine oxide, 2,4,6-trimethylbenzoylphenylphosphinic acid ethyl ester, bis(2,4,4-trimethylpentyl)(2,6-dimethoxybenzoyl)phosphine oxide, bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide, bis-(2-chloro-1-naphthoyl)-2,5-dimethylphenylphosphine oxide and combinations thereof.
120. The method according to any of clauses 101 to 116, wherein the color changer is a singlet oxygen photosensitizer.
121. The method according to clause 120 wherein the singlet oxygen photosensitizer absorbs light of 400 nm or more.
122. The method according to clause 120 or 121, wherein the singlet oxygen photosensitizer is a polycyclic aromatic hydrocarbon, cyanine, fluroscein, anthracene or porphyrin.
123. The method according to any of clauses 120 to 122, wherein the singlet oxygen photosensitizer is selected from the group consisting of acridine, acetonaphthone, anthra[1,9-bc:4,10-b′c′]dichromene, 9,10-anthracenedipropionate ion, aluminum(III) sulfophthalocyanine, anthracene, angelicin, anthracenesulfonate ion, acetophenone, 9,10-athraquinone, allylthiourea, bacteriochlorophyll a, benzo[1,2,3-kl:4,5,6k′l′]dixanthene, biphenyl, benzophenone, bilirubin, bilirubin dianion, benoxaprofen, β-carotene, cadmium(II) 1-(2-hydroxyphenylazo)-2-naphtholate, chlorophyll a, camphoroquinone, chlorpromazine, 9,10-dicyanoanthracene, 9,10-dimethylanthracene, 4,7-dimethylallopsoralen, 9,10-dimethylbenz[a]anthracene, 1,4-dimethoxy-9,10-diphenylanthracene, 2,5-dimethylfuran, 4,4′-dimethoxythiobenzophenone, 1,8-dinaphthalene thiophene, diacenaphtho[1,2-b:1′,2′-d]thiophene, 3-(3,4-dihydroxyphenyl)alanine, 9,10-diphenylanthracene, 1,4-diphenyl-1,3-butadiene, 1,3-diphenylisobenzofuran, 2,5-diphenylfuran, 1,6-diphenyl-1,3,5-hexatriene, 1,8-diphenyl-1,3,5,7-octatetraene, 2,5-di-tert-butylfuran, eosin (tetrabromofluorescein), erythrosin (tetraiodofluorescein), ergosterol, furfuryl alcohol, fluorescein, heterocoerdianthrone, histidine, hematoporphyrin, hypericin, imidazole, 4′-methoxyacetophenone, methylene blue, mesodiphenylbenzhelianthrene, mesodiphenylhelianthrene, 1-methylnaphthalene, methoxypsoralen, 2-methyl-2-pentene, mesoporphyrin diethyl ester, mesoporphyrin dimethyl ester, 10-Methyl-9-acridinethione, naphthalene, palladium(II) tetraphenylporphyrin, palladium(II) tetrakis(4-sulfonatophenyl)porphyrin, perylene, pheophytin a, protoporphyrin, protoporphyrin dimethyl ester, 2,7,12,17-tetrapropylporphycene, platinum(II) diazido(2,2′-bipyridine), platinum(II) (1,10-phenanthroline)(tert-butylcatechol), platinum(II) (1,10-phenanthroline)(2,3-naphthalenediol), pyrene, phenazine, Rose Bengal (tetrachlorotetraiodofluorescein), Rose Bengal ethyl ester, retinal, riboflavin, N,N-dimethyl-4-nitrosoaniline, rubrene (5,6,11,12-tetraphenylnaphthacene), 2,2,6,6-tetramethylpiperidin-4-ol, tetracene, tetra(3-hydroxyphenyl)porphyrin, tetra(4-hydroxyphenyl)porphyrin, 2,3-Dimethyl-2-butene (tetramethylethylene), tetra(4-N-methylpyridyl)porphyrin, tetraphenylbacteriochlorin, tetraphenylcyclopentadienone, tetraphenylporphyrin, tetra(4-sulfonatophenyl)porphyrin, tryptophan, uroporphyrin I, zinc(II) tetraphenylporphyrin, zinc(II) 2-(4,5-diphenylimidazol-2-yl)azo-5-methylbenzoate and zinc(II) 2-(4,5-diphenylimidazol-2-yl)azo-4-nitrophenolate and combinations thereof.
124. The method according to any of clauses 101 to 123, wherein the color changer initiates the decomposition of the colored pigment.
125. The method according to clause 124, wherein the colored pigment decomposes in 10 minutes or less after application of the applied stimulus.
126. The method according to clause 124, wherein the colored pigment decomposes in 5 minutes or less after application of the applied stimulus.
127. The method according to any of clauses 101 to 126, wherein the colored pigment and color changer are microencapsulated.
128. The method according to clause 127, wherein the colored pigment and color changer are microencapsulated with a maleic anhydride copolymer or a urea formaldehyde resin.
129. The method according to any of clauses 101 to 128, further comprising a solvent.
130. The method according to clause 129, wherein the solvent is a long chain hydrocarbon alcohol.
131. The method according to clause 130, wherein the long chain hydrocarbon alcohol is selected from the group consisting of 1-tetradecanol, 1-hexadecanol, 1-octadecanol, 1-eicosanol, 1-docosanol, oils, mineral oil, petroleum jelly, corn oil, canola oil, castor oil, and mixtures thereof.
132. The method according to any of clauses 101 to 131, wherein the molar ratio of color changer to colored pigment is from 0.1 to 50.
133. The method according to clause 132, wherein the molar ratio of color changer to colored pigment is 10.
134. A method for forming a uniform coating on a surface, the method comprising applying to the surface an evanescent color change composition comprising:

a colored pigment; and

a color changer that reversibly changes the colored pigment from colored to colorless in response to light.
135. The method according to Clause 134, further comprising visualizing that the applied color change composition is uniformly coated onto the surface.
136. The method according to Clause 134, wherein applying comprises brushing the composition onto the support.
137. The method according to Clause 134, wherein the support is a skin of a subject.
138. The method according to Clause 137, wherein applying comprises manually coating the skin of the subject with the composition.
139. The method according to any of clauses 134 to 138, wherein the colored pigment is a dye.
140. The method according to clause 139, wherein the colored pigment is a hydrophobic dye.
141. The method according to clause 139, wherein the colored pigment is an anthraquinone dye.
142. The method according to clause 139, wherein the colored pigment is an oil soluble dye.
143. The method according to clause 139, wherein the colored pigment is a carotenoid.
144. The method according to any of clauses 134 to 143, wherein the colored pigment is chemically coupled to a long chain hydrocarbon.
145. The method according to any of clauses 134 to 143, wherein the colored pigment is chemically coupled to a polyalkylene glycol.
146. The method according to clause 134, wherein the light is ultraviolet (UV) light or visible light.
147. The method according to clause 146, wherein the light has a wavelength from 200 nm to 400 nm.
148. The method according to clause 146, wherein the light has a wavelength from 400 nm to 700 nm.
149. The method according to clause 146, wherein the light has a wavelength from 700 nm to 1200 nm.
150. The method according to any of clauses 134 to 149, wherein the color changer is a free radical photoinitiator.
151. The method according to clause 150, wherein the free radical photoinitiator is a phosphine oxide, an α-amino ketone photoinitiator, a titanocene and an azide compound.
152. The method according to clause 151, wherein the free radical photoinitiator comprises a phosphine oxide selected from the group consisting of bis(2,6-dimethoxybenzoyl)-(2-methylpropyl)phosphine oxide, bis(2,4,6-trimethylbenzoyl)-(2-methylpropyl)phosphine oxide, bis(2,6-dimethoxybenzoyl)-(2,4,4-trimethylpentyl)phosphine oxide, bis(2,4,6-trimethylbenzoyl)-(2,4,4-trimethylpentyl)phosphine oxide, (2,6-dimethoxybenzoyl)-(2,4,6-trimethylbenzoyl)-(2-methylpropyl)phosphine oxide, phenylbis(3-{[2-(allyloxy)ethoxy]methyl}-2,4,6-trimethylbenzoyl)phosphine oxide, phenylbis{4-[2-(2-Methoxy-ethoxy)-ethoxy]-2,6-dimethyl-benzoyl}phosphine oxide, phenylbis{3-[2-(2-Methoxy-ethoxy)-ethoxymethyl]-2,4,6-trimethyl-benzoyl}phosphine oxide, phenylbis(2,4,6-trimethyl-benzoyl)-4-[2-(2-methoxyethoxy)-ethoxy]phosphine oxide, phenylbis(2,4,6-trimethyl-benzoyl)-4-methoxy-phosphine oxide, phenylbis(2,4,6-trimethylbenzoyl)-phosphine oxide, 2,4,6-trimethylbenzoyldiphenylphosphine oxide, 2,4,6-trimethylbenzoylphenyl phosphinate, bis(2-methylpropyl)(2,6-dimethoxybenzoyl)phosphine oxide, bis(2-methylpropyl)(2,4,6-trimethylbenzoyl)phosphine oxide, 2,4,6-trimethylbenzoylphenylphosphinic acid ethyl ester, bis(2,4,4-trimethylpentyl)(2,6-dimethoxybenzoyl)phosphine oxide, bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide, bis-(2-chloro-1-naphthoyl)-2,5-dimethylphenylphosphine oxide and combinations thereof.
153. The method according to any of clauses 134 to 149, wherein the color changer is a singlet oxygen photosensitizer.
154. The method according to clause 153, wherein the singlet oxygen photosensitizer absorbs light of 400 nm or more.
155. The method according to clause 153 or 154, wherein the singlet oxygen photosensitizer is a polycyclic aromatic hydrocarbon, cyanine, fluroscein, anthracene or porphyrin.
156. The method according to any of clauses 153 to 155, wherein the singlet oxygen photosensitizer is selected from the group consisting of acridine, acetonaphthone, anthra[1,9-bc:4,10-b′c′]dichromene, 9,10-anthracenedipropionate ion, aluminum(III) sulfophthalocyanine, anthracene, angelicin, anthracenesulfonate ion, acetophenone, 9,10-athraquinone, allylthiourea, bacteriochlorophyll a, benzo[1,2,3-kl:4,5,6k′l′]dixanthene, biphenyl, benzophenone, bilirubin, bilirubin dianion, benoxaprofen, β-carotene, cadmium(II) 1-(2-hydroxyphenylazo)-2-naphtholate, chlorophyll a, camphoroquinone, chlorpromazine, 9,10-dicyanoanthracene, 9,10-dimethylanthracene, 4,7-dimethylallopsoralen, 9,10-dimethylbenz[a]anthracene, 1,4-dimethoxy-9,10-diphenylanthracene, 2,5-dimethylfuran, 4,4′-dimethoxythiobenzophenone, 1,8-dinaphthalene thiophene, diacenaphtho[1,2-b:1′,2′-d]thiophene, 3-(3,4-dihydroxyphenyl)alanine, 9,10-diphenylanthracene, 1,4-diphenyl-1,3-butadiene, 1,3-diphenylisobenzofuran, 2,5-diphenylfuran, 1,6-diphenyl-1,3,5-hexatriene, 1,8-diphenyl-1,3,5,7-octatetraene, 2,5-di-tert-butylfuran, eosin (tetrabromofluorescein), erythrosin (tetraiodofluorescein), ergosterol, furfuryl alcohol, fluorescein, heterocoerdianthrone, histidine, hematoporphyrin, hypericin, imidazole, 4′-methoxyacetophenone, methylene blue, mesodiphenylbenzhelianthrene, mesodiphenylhelianthrene, 1-methylnaphthalene, methoxypsoralen, 2-methyl-2-pentene, mesoporphyrin diethyl ester, mesoporphyrin dimethyl ester, 10-Methyl-9-acridinethione, naphthalene, palladium(II) tetraphenylporphyrin, palladium(II) tetrakis(4-sulfonatophenyl)porphyrin, perylene, pheophytin a, protoporphyrin, protoporphyrin dimethyl ester, 2,7,12,17-tetrapropylporphycene, platinum(II) diazido(2,2′-bipyridine), platinum(II) (1,10-phenanthroline)(tert-butylcatechol), platinum(II) (1,10-phenanthroline)(2,3-naphthalenediol), pyrene, phenazine, Rose Bengal (tetrachlorotetraiodofluorescein), Rose Bengal ethyl ester, retinal, riboflavin, N,N-dimethyl-4-nitrosoaniline, rubrene (5,6,11,12-tetraphenylnaphthacene), 2,2,6,6-tetramethylpiperidin-4-ol, tetracene, tetra(3-hydroxyphenyl)porphyrin, tetra(4-hydroxyphenyl)porphyrin, 2,3-Dimethyl-2-butene (tetramethylethylene), tetra(4-N-methylpyridyl)porphyrin, tetraphenylbacteriochlorin, tetraphenylcyclopentadienone, tetraphenylporphyrin, tetra(4-sulfonatophenyl)porphyrin, tryptophan, uroporphyrin I, zinc(II) tetraphenylporphyrin, zinc(II) 2-(4,5-diphenylimidazol-2-yl)azo-5-methylbenzoate and zinc(II) 2-(4,5-diphenylimidazol-2-yl)azo-4-nitrophenolate and combinations thereof.
157. The method according to any of clauses 134 to 156, wherein the color changer initiates the decomposition of the colored pigment.
158. The method according to clause 157, wherein the colored pigment decomposes in 10 minutes or less after application of the applied stimulus.
159. The method according to clause 157, wherein the colored pigment decomposes in 5 minutes or less after application of the applied stimulus.
160. The method according to any of clauses 134 to 159, wherein the colored pigment and color changer are microencapsulated.
161. The method according to clause 160, wherein the colored pigment and color changer are microencapsulated with a maleic anhydride copolymer or a urea formaldehyde resin.
162. The method according to any of clauses 134 to 161, further comprising a solvent.
163. The method according to clause 161, wherein the solvent is a long chain hydrocarbon alcohol.
164. The method according to clause 163, wherein the long chain hydrocarbon alcohol is selected from the group consisting of 1-tetradecanol, 1-hexadecanol, 1-octadecanol, 1-eicosanol, 1-docosanol, oils, mineral oil, petroleum jelly, corn oil, canola oil, castor oil, and mixtures thereof.
165. The method according to any of clauses 134 to 164, wherein the molar ratio of color changer to colored pigment is from 0.1 to 50.
166. The method according to clause 165, wherein the molar ratio of color changer to colored pigment is 10.
167. An evanescent color change sunscreen composition comprising:

a liquid dispersion comprising a UV absorber; and

an evanescent color change composition comprising:
a colored pigment; and
a color changer that changes the colored pigment from colored to colorless in response to light.
168. The composition according to clause 167, wherein the UV absorber is a UV-A absorber.
169. The composition according to clause 167, wherein the UV absorber is a UV-B absorber.
170. The composition according to clause 167, wherein the UV absorber is a UV-C absorber.
171. The composition according to clause 167, wherein the UV absorber comprises titanium dioxide, zinc oxide, p-Aminobenzoic acid (PABA), octyldimethyl-PABA, phenylbenzimidazole sulfonic acid, 2-ethoxyethyl p-methoxycinnamate, dioxybenzone, oxybenzone, homomethyl salicylate, menthyl anthranilate, 2-cyano-3,3-diphenyl acrylic acid 2-ethylhexylester, 2-ethylhexyl-paramethoxycinnamate, 2-ethylhexyl salicylate, 3-benzoyl-4-hydroxy-6-methoxybenzenesulfonic acid, triethanolamine salicylate, 1-(4-methoxyphenyl)-3-(4-tert-butyl phenyl)propane-1,3-dione, terephthalylidene dicamphor sulfonic acid, 4-methylbenzylidene camphor, methylene bis-benzotriazolyl tetramethylbutylphenol, tris-biphenyl triazine, disodium phenyl dibenzimidazole tetrasulfonate, drometrizole trisiloxane, sodium dihydroxy dimethoxy disulfobenzophenone, ethylhexyl triazone, diethylamino hydroxybenzoyl hexyl benzoate, diethylhexyl butamido triazone, dimethico-diethylbenzalmalonate, isopentyl-4-methoxycinnamate or a combination or mixture thereof.
172. The composition according to any of clauses 167 to 171, wherein the colored pigment is a dye.
173. The composition according to clause 172, wherein the colored pigment is a hydrophobic dye.
174. The composition according to clause 172, wherein the colored pigment is an anthraquinone dye.
175. The composition according to clause 172, wherein the colored pigment is an oil soluble dye.
176. The composition according to clause 172, wherein the colored pigment is a carotenoid.
177. The composition according to any of clauses 167 to 176, wherein the colored pigment is chemically coupled to a long chain hydrocarbon.
178. The composition according to any of clauses 167 to 176, wherein the colored pigment is chemically coupled to a polyalkylene glycol.
179. The composition according to clause 178, wherein the light is ultraviolet (UV) light or visible light.
180. The composition according to clause 178, wherein the light has a wavelength from 200 nm to 400 nm.
181. The composition according to clause 178, wherein the light has a wavelength from 400 nm to 700 nm.
182. The composition according to clause 178, wherein the light has a wavelength from 700 nm to 1200 nm.
183. The composition according to any of clauses 167 to 182, wherein the color changer is a free radical photoinitiator.
184. The composition according to clause 183, wherein the free radical photoinitiator is a phosphine oxide, an α-amino ketone photoinitiator, a titanocene and an azide compound.
185. The composition according to clause 184, wherein the free radical photoinitiator comprises a phosphine oxide selected from the group consisting of bis(2,6-dimethoxybenzoyl)-(2-methylpropyl)phosphine oxide, bis(2,4,6-trimethylbenzoyl)-(2-methylpropyl)phosphine oxide, bis(2,6-dimethoxybenzoyl)-(2,4,4-trimethylpentyl)phosphine oxide, bis(2,4,6-trimethylbenzoyl)-(2,4,4-trimethylpentyl)phosphine oxide, (2,6-dimethoxybenzoyl)-(2,4,6-trimethylbenzoyl)-(2-methylpropyl)phosphine oxide, phenylbis(3-{[2-(allyloxy)ethoxy]methyl}-2,4,6-trimethylbenzoyl)phosphine oxide, phenylbis{4-[2-(2-Methoxy-ethoxy)-ethoxy]-2,6-dimethyl-benzoyl}phosphine oxide, phenylbis{3-[2-(2-Methoxy-ethoxy)-ethoxymethyl]-2,4,6-trimethyl-benzoyl}phosphine oxide, phenylbis(2,4,6-trimethyl-benzoyl)-4-[2-(2-methoxyethoxy)-ethoxy]phosphine oxide, phenylbis(2,4,6-trimethyl-benzoyl)-4-methoxy-phosphine oxide, phenylbis(2,4,6-trimethylbenzoyl)-phosphine oxide, 2,4,6-trimethylbenzoyldiphenylphosphine oxide, 2,4,6-trimethylbenzoylphenyl phosphinate, bis(2-methylpropyl)(2,6-dimethoxybenzoyl)phosphine oxide, bis(2-methylpropyl)(2,4,6-trimethylbenzoyl)phosphine oxide, 2,4,6-trimethylbenzoylphenylphosphinic acid ethyl ester, bis(2,4,4-trimethylpentyl)(2,6-dimethoxybenzoyl)phosphine oxide, bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenylphosphine oxide, bis-(2,6-dichlorobenzoyl)-4-biphenylylphosphine oxide, bis-(2,6-dichlorobenzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenylphosphine oxide, bis-(2,6-dichlorobenzoyl)-2,4-dimethoxyphenylphosphine oxide, bis-(2,6-dichlorobenzoyl)decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenyl phosphine oxide, bis-(2,6-dichloro-3,4,5-trimethoxybenzoyl)-4-ethoxyphenylphosphine oxide, bis-(2-methyl-1-naphthoyl)-2,5-dimethylphenylphosphine oxide, bis-(2-methyl-1-naphthoyl)phenylphosphine oxide, bis-(2-methyl-1-naphthoyl)-4-biphenylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide, bis-(2-chloro-1-naphthoyl)-2,5-dimethylphenylphosphine oxide and combinations thereof.
186. The sunscreen composition according to any of clauses 167 to 182, wherein the color changer is a singlet oxygen photosensitizer.
187. The composition according to clause 186 wherein the singlet oxygen photosensitizer absorbs light of 400 nm or more.
188. The composition according to clause 186 or 187, wherein the singlet oxygen photosensitizer is a polycyclic aromatic hydrocarbon, cyanine, fluroscein, anthracene or porphyrin.
189. The composition according to any of clauses 186 to 188, wherein the singlet oxygen photosensitizer is selected from the group consisting of acridine, acetonaphthone, anthra[1,9-bc:4,10-b′c′]dichromene, 9,10-anthracenedipropionate ion, aluminum(III) sulfophthalocyanine, anthracene, angelicin, anthracenesulfonate ion, acetophenone, 9,10-athraquinone, allylthiourea, bacteriochlorophyll a, benzo[1,2,3-kl:4,5,6k′l′]dixanthene, biphenyl, benzophenone, bilirubin, bilirubin dianion, benoxaprofen, β-carotene, cadmium(II) 1-(2-hydroxyphenylazo)-2-naphtholate, chlorophyll a, camphoroquinone, chlorpromazine, 9,10-dicyanoanthracene, 9,10-dimethylanthracene, 4,7-dimethylallopsoralen, 9,10-dimethylbenz[a]anthracene, 1,4-dimethoxy-9,10-diphenylanthracene, 2,5-dimethylfuran, 4,4′-dimethoxythiobenzophenone, 1,8-dinaphthalene thiophene, diacenaphtho[1,2-b:1′,2′-d]thiophene, 3-(3,4-dihydroxyphenyl)alanine, 9,10-diphenylanthracene, 1,4-diphenyl-1,3-butadiene, 1,3-diphenylisobenzofuran, 2,5-diphenylfuran, 1,6-diphenyl-1,3,5-hexatriene, 1,8-diphenyl-1,3,5,7-octatetraene, 2,5-di-tert-butylfuran, eosin (tetrabromofluorescein), erythrosin (tetraiodofluorescein), ergosterol, furfuryl alcohol, fluorescein, heterocoerdianthrone, histidine, hematoporphyrin, hypericin, imidazole, 4′-methoxyacetophenone, methylene blue, mesodiphenylbenzhelianthrene, mesodiphenylhelianthrene, 1-methylnaphthalene, methoxypsoralen, 2-methyl-2-pentene, mesoporphyrin diethyl ester, mesoporphyrin dimethyl ester, 10-Methyl-9-acridinethione, naphthalene, palladium(II) tetraphenylporphyrin, palladium(II) tetrakis(4-sulfonatophenyl)porphyrin, perylene, pheophytin a, protoporphyrin, protoporphyrin dimethyl ester, 2,7,12,17-tetrapropylporphycene, platinum(II) diazido(2,2′-bipyridine), platinum(II) (1,10-phenanthroline)(tert-butylcatechol), platinum(II) (1,10-phenanthroline)(2,3-naphthalenediol), pyrene, phenazine, Rose Bengal (tetrachlorotetraiodofluorescein), Rose Bengal ethyl ester, retinal, riboflavin, N,N-dimethyl-4-nitrosoaniline, rubrene (5,6,11,12-tetraphenylnaphthacene), 2,2,6,6-tetramethylpiperidin-4-ol, tetracene, tetra(3-hydroxyphenyl)porphyrin, tetra(4-hydroxyphenyl)porphyrin, 2,3-Dimethyl-2-butene (tetramethylethylene), tetra(4-N-methylpyridyl)porphyrin, tetraphenylbacteriochlorin, tetraphenylcyclopentadienone, tetraphenylporphyrin, tetra(4-sulfonatophenyl)porphyrin, tryptophan, uroporphyrin I, zinc(II) tetraphenylporphyrin, zinc(II) 2-(4,5-diphenylimidazol-2-yl)azo-5-methylbenzoate and zinc(II) 2-(4,5-diphenylimidazol-2-yl)azo-4-nitrophenolate and combinations thereof.
190. The composition according to any of clauses 167 to 189, wherein the color changer initiates the decomposition of the colored pigment.
191. The composition according to clause 190, wherein the colored pigment decomposes in 10 minutes or less after application of the applied stimulus.
192. The composition according to clause 190, wherein the colored pigment decomposes in 5 minutes or less after application of the applied stimulus.
193. The composition according to any of clauses 167 to 192, wherein the colored pigment and color changer are microencapsulated.
194. The composition according to clause 193, wherein the colored pigment and color changer are microencapsulated with a maleic anhydride copolymer or a urea formaldehyde resin.
195. The composition according to any of clauses 167 to 194, further comprising a solvent.
196. The composition according to clause 195, wherein the solvent is a long chain hydrocarbon alcohol.
197. The composition according to clause 196, wherein the long chain hydrocarbon alcohol is selected from the group consisting of 1-tetradecanol, 1-hexadecanol, 1-octadecanol, 1-eicosanol, 1-docosanol, oils, mineral oil, petroleum jelly, corn oil, canola oil, castor oil, and mixtures thereof.
198. The composition according to any of clauses 167 to 197, wherein the molar ratio of color changer to colored pigment is from 0.1 to 50.
199. The composition according to clause 198, wherein the molar ratio of color changer to colored pigment is 10.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An evanescent color change composition comprising:
a colored pigment; and
a color changer that changes the colored pigment from colored to colorless in response to an applied stimulus to the color changer.

2. The color change composition according to claim 1, wherein the colored pigment is a dye.

3. The color change composition according to claims 1 or 2, wherein the color changer changes the colored pigment from colored to colorless in response to light.

4. The color change composition according to claim 3, wherein the light is ultraviolet (UV) light or visible light.

5. The color change composition according to any of claims 1 to 4, wherein the color changer is a free radical photoinitiator or a singlet oxygen photosensitizer.

6. The color change composition according to any of claims 1 to 5, wherein the color changer initiates the decomposition of the colored pigment.

7. The color change composition according to any of claims 1 to 6, wherein the colored pigment and color changer are microencapsulated.

8. The color change composition according to claim 7, wherein the colored pigment and color changer are microencapsulated with a maleic anhydride copolymer or a urea formaldehyde resin.

9. The color change composition according to any of claims 1 to 8, further comprising a solvent.

10. The color change composition according to any of claims 1 to 9, further comprising a UV absorber.

11. A liquid evanescent color change composition comprising:
a solvent; and
a color change composition according to any of claims 1 to 10.

12. A method of making an evanescent color change composition a color change composition according to any of claims 1 to 10, the method comprising:
combining a colored pigment with a color change that changes the colored pigment from colored to colorless upon application of an applied stimulus to the color changer.

13. A method of making an evanescent color change sunscreen composition, the method comprising combining a dispersion comprising a UV absorber with an evanescent color change composition according to any of claims 1 to 10 to form the color change sunscreen composition.

14. A method for forming a uniform coating on a surface, the method comprising applying to the surface an evanescent color change composition according to any of claims 1 to 10.

15. An evanescent color change sunscreen composition comprising:
a liquid dispersion comprising a UV absorber; and
an evanescent color change composition according to any of claims 1 to 10.
